(19) United States
(12) Patent Application Publication
(10) Pub. No.: US 2025/0313846 A1
(43) Pub. Date: Oct. 9, 2025

(54) PRODUCTS AND COMPOSITIONS

(71) Applicant: Sirnaomics, Inc., Germantown, MD (US)

(72) Inventors: Dmitry SAMARSKY, Germantown, MD (US); Jack WEI, Germantown, MD (US)

(21) Appl. No.: 18/977,340

(22) Filed: Dec. 11, 2024

Related U.S. Application Data
(63) Continuation of application No. PCT/US2023/068218, filed on Jun. 9, 2023.
(60) Provisional application No. 63/351,357, filed on Jun. 11, 2022.

(51) Int. Cl. C12N 15/113

(52) U.S. Cl. CPC C12N 15/1138; C12N 15/113; C12N 2310/14; C12N 2310/316; C12N 2310/321; C12N 2310/322; C12N 2310/333; C12N 2310/3341; C12N 2310/335; C12N 2310/336; C12N 2310/351

(57) ABSTRACT

Nucleic acid products are provided that modulate, in particular interfere with or inhibit, TMPRSS6 and APOC3 gene expression.

### RELATED APPLICATIONS

The present application is a continuation of International Application No. PCT/US2023/068218, filed on Jun. 9, 2023, which claims the benefit of and priority to US Provisional Patent Application No. This application claims the benefit of and priority to U.S. Provisional Patent Application No. 63/351,357, filed on Jun. 11, 2022, both of which are incorporated herein by reference in their entireties.

### SEQUENCE LISTING

The instant application contains a Sequence Listing that has been submitted electronically in ST.26 XML format and is hereby incorporated by reference in its entirety. The XML file was created on Jun. 6, 2023, is named 4690_0071I_SL and is 3060 kilobytes in size.

### FIELD

The present disclosure relates to products, and compositions, and their uses. In particular, the present disclosure relates to nucleic acid products that modulate, in particular interfere with or inhibit TMPRSS6 and APOC3 gene expression. Embodiments of the present disclosure can therefore provide methods, compounds, and compositions for reducing expression of TMPRSS6 and APOC3 mRNA and protein in an animal. Such methods, compounds, and compositions are useful to treat, prevent, or ameliorate TMPRSS6- and APOC3-associated disorders such as iron overload or hemochromatosis, and dyslipidaemia.

### BACKGROUND

While iron is an essential mineral, failure of its regulation, multiple transfusions, excessive intake, or disorders including genetic disorders may lead to iron overload—an excess of iron stored in the liver, heart and pancreas that can cause life-threatening conditions if left untreated. Iron overload occurs, for example, in patients suffering from hemochromatosis, an inherited disease.

TMPRSS6 (Transmembrane protease, serine 6; also known as matriptase-2) is an enzyme which is inter alia involved in iron ion homeostasis. It is highly expressed in the liver. TMPRSS6 downregulates hepcidin, the key regulator of iron homeostasis. Since low levels of hepcidin correlate with iron overload, inhibiting the expression of the TMPRSS6 gene is an approach for mitigating iron overload and its associated disorders and diseases.

Triglycerides are esters of glycerol with three fatty acids. They serve as storage of fat and energy and are transported via the bloodstream. Excess level of blood triglycerides have been recognized early on as causative agents or bystanders of a range of disorders. More recent evidence suggests a causative role, partly in conjunction with elevated levels of cholesterol (in particular LDL cholesterol) in ASCVD and related disorders and diseases. A more comprehensive list of disorders associated with elevated levels of triglycerides is given in the embodiments disclosed below.

Apolipoprotein C3 (APOC3) is secreted by the liver and the small intestine. It can be found on triglyceride-rich lipoproteins including very low density lipoproteins (VLDL) and chylomicrons. It is involved in the negative regulation of lipid catabolism, especially triglyceride catabolism, and of the clearance of VLDL, LDL and HDL lipoproteins. APOC3 inhibits lipoprotein lipase and hepatic lipase.

### Disorders and Diseases

Iron overload, as it occurs for example in hemochromatosis, may contribute to the development of various disorders and diseases including diabetes, glucose intolerance, cardiovascular diseases, hepatic injury, and steatohepatitis, and may even be lethal.

Further, Hypertriglyceridemia (HTG), which refers to excessive levels of circulating triglycerides, is a recognized disorder in itself and is associated with inflammation and cardiovascular disorders and diseases, particularly when HTG persists over extended periods . . .

Evidence exists that iron overload or hemochromatosis on the one hand and HTG on the other hand co-occur; see, for example, Casanova-Esteban et al., Metabolism 60, 830-834 (2011), and Silva et al., Nutrition Research 28, 391-398 (2008). Accordingly, a treatment combining an inhibitor of TMPRSS6 with an inhibitor of APOC3 may benefit subjects afflicted with these iron- and lipid-related disorders or diseases.

### Treatment

In view of the potentially severe consequences, there remains a need for therapies to treat iron overload, lipid dysregulation and associated diseases including TMPRSS6- and APOC3-associated diseases. One aim of this disclosure is to provide compounds, methods, and pharmaceutical compositions for the treatment of such diseases and disorders.

Double-stranded RNA (dsRNA) capable of complementarily binding expressed mRNA has been shown to block gene expression (Fire et al., 1998, Nature. 1998 Feb. 19; 391 (6669): 806-1 1 and Elbashir et at., 2001, Nature. 2001 May 24; 41 1 (6836): 494-8) by an RNA interference (RNAi) mechanism. Short dsRNAs direct gene-specific, post-transcriptional silencing in many organisms, including vertebrates, and have become a useful tool for studying gene function. RNAi is mediated by the RNA-induced silencing complex (RISC), a sequence-specific, multi-component nuclease that destroys messenger RNAs homologous to the silencing trigger loaded into the RISC complex. Interfering RNA (iRNA) such as small interfering RNA (siRNAs), antisense RNA (asRNA), and micro-RNA (miRNA) are oligonucleotides that prevent the formation of proteins by gene-silencing i.e. inhibiting gene translation of the protein through degradation of mRNA molecules. Gene-silencing agents are becoming increasingly important for therapeutic applications in medicine.

The discovery of potent gene-silencing agents with minimal, off-target effects is a complex process. Although algorithms can be used to design gene-silencing triggers agents such as siRNA, there are limitations. These include a failure of the algorithms to account for the tertiary structure of the target mRNA and for the involvement of RNA binding proteins (Watts & Corey. J Pathol. 226:365-379, 2012). These highly charged molecules used in pharmaceutical compositions should be capable of (i) being synthesized economically; (ii) being distributed to target tissues; (iii) entering cells; and (iv) functioning within acceptable limits of toxicity. Another aim of this disclosure is, therefore, to provide compounds, methods, and pharmaceutical compositions for the treatment of TMPRSS6- and APOC3-related disorders and diseases using oligomeric compounds that modulate, in particular inhibit, gene expression by RNAi.

### SUMMARY

The present disclosure relates to nucleic acid products that modulate, in particular, interfere with or inhibit, TMPRSS6 and APOC3 gene expression, and associated therapeutic uses. Specific oligomeric compounds and sequences according to the present disclosure are described herein. This summary is provided to introduce the disclosure in a simplified form that is further described below in the detailed description. This summary is not intended to identify key features or essential features of the claimed subject matter, nor is it intended to be used to determine the scope of the claimed subject matter.

The present disclosure provides the following non-limiting aspects.

According to a first aspect, the present disclosure is directed to nucleic acid construct comprising at least:
(a) a first nucleic acid portion that is at least partially complementary to at least a first portion of an RNA which is transcribed from a TMPRSS6 gene;
(b) a second nucleic acid portion that is at least partially complementary to at least a second portion of an RNA which is transcribed from a APOC3 gene;
(c) a third nucleic acid portion that is at least partially complementary to the first nucleic acid portion of (a), so as to form a first nucleic acid duplex region therewith;
(d) a fourth nucleic acid portion that is at least partially complementary to the second nucleic acid portion of (b), so as to form a second nucleic acid duplex region therewith.

According to a second aspect, the present disclosure is directed to a composition comprising a construct according to the first aspect, and a physiologically acceptable excipient.

According to a third aspect, the present disclosure is directed to a pharmaceutical composition comprising a construct according to the first aspect.

According to a fourth aspect, the present disclosure is directed to a construct according to the first aspect, for use in human or veterinary medicine or therapy.

According to a fifth aspect, the present disclosure is directed to a construct according to the first aspect, for use in a method of treating a disease or disorder.

According to a sixth aspect, the present disclosure is directed to a method of treating a disease or disorder comprising administration of a construct according to the first aspect, to an individual in need of treatment.

According to a seventh aspect, the present disclosure is directed to a use of a nucleic acid construct according to the first aspect in the manufacture of a medicament for a treatment of a disease or disorder.

According to an eight aspect, the present disclosure is directed to a use of a construct according to the first aspect, for use in research as a gene function analysis tool.

According to a nineth aspect, the present disclosure is directed to a process of making a construct according to the first aspect.

Further embodiments (items; claims) of the present disclosure are described below by way of example only. These examples represent the best ways of putting the disclosure into practice that are currently known to the applicant although they are not the only ways in which this could be achieved.

It will be understood that the benefits and advantages described herein may relate to one embodiment or may relate to several embodiments. The embodiments are not limited to those that solve any or all of the stated problems or those that have any or all of the stated benefits and advantages.

Features of different aspects and embodiments of the disclosure may be combined as appropriate, as would be apparent to a skilled person, and may be combined with any of the aspects of the disclosure.

Optional and/or exemplary features of constructs according to the present disclosure are as follows:
1) they contain multiple (2 or more) at least partially double-stranded agents capable of triggering RNA interference, tied together into a single nano-structure predominantly through complementary (Watson-Crick) interactions;
2) optionally, other (e.g.) covalent bindings may be used to build the constructs and/or add various ligands (e.g. delivery/targeting moieties such as GalNAc and or other carbohydrates, cholesterol, peptides, or small molecules, optionally attached via linkers);
3) the constructs of the disclosure predominantly comprise chemically modified nucleotides (e.g. 2′F, 2′OMe, LNO, PNA, MOE, BNA, PMO, phosphorothioate, phosphodithioate, etc.etc), mostly (but not only) to increase resistance to nucleases;
4) the constructs contain “fragile” components (e.g. chemical linkers, unmodified nucleotides, etc), which allow the constructs to disassemble upon exposure to certain biologic environments (e.g. exposure to extra- and/or intra-cellular fluids); particular examples could be (but not limited): a) cleavage of the oligo backbone by nucleases in the sites with non-modified nucleotides; b) cleavage of the chemical linkage due to the change of pH (e.g. in endosomes);
5) disassembly upon exposure to the certain biologic environments releases the active components (e.g. the at least partially double-stranded agents capable of triggering RNA interference) to modulate (up- or down-regulate, optionally down-regulate) target gene expression in cells/organisms;
6) the constructs can be used to modulate, optionally down-regulate or silence gene expression, to study gene function, or to treat various diseases associated with the target genes to be down-regulated.

### BRIEF DESCRIPTION OF THE FIGURES

FIG. 1 shows a schematic overview of the design of the in vivo study.

FIG. 2 shows knockdown of TMPRSS6 and APOC3 mRNA in liver tissue.

FIG. 3 shows a comparison of APOC3 mRNA knockdown in liver tissue with APOC3 protein knockdown in plasma, demonstrating a high correlation between the two parameters.

FIG. 4 shows a comparison of a single treatment with multiple treatment (see the study design in FIG. 1). Results are comparable, wherein a further increase of TMPRSS6 mRNA knockdown is observed for multiple treatment.

FIG. 5 shows the effect on TMPRSS6 mRNA levels in both normal mouse and mice with a humanized liver. The humanized mouse liver still retains a certain fraction of murine liver cells. Since a construct has been employed which is capable of knocking down both human and murine TMPRSS6, all three read-outs shown demonstrate knockdown of the respective TMPRSS6 mRNA.

FIG. 6 shows a concentration dependence of 5 TMPRSS6 muRNA sequences and their TMPRSS6 in vitro inhibition by certain mxRNA constructs of Table 7a.

FIG. 7 shows a concentration dependence of 5 TMPRSS6 muRNA sequences and their APCO3 in vitro inhibition by certain mxRNA constructs of Table 7a.

### DETAILED DESCRIPTION AND EMBODIMENTS

Further implementations of the present disclosure are described below by way of example only. These examples represent the advantageous ways of putting the disclosure into practice that are currently known to the applicant although they are not the only ways in which this could be achieved.

Features of different aspects and implementations or embodiments may be combined as appropriate, as would be apparent to a skilled person.

### Definitions

The following definitions pertain to the disclosure throughout. In many instances, the definitions, in addition to the respective definition as such, provide non-exhaustive listings of possible implementations which amount to optional embodiments.

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21st edition, 2005; and “Antisense Drug Technology, Principles, Strategies, and Applications” Edited by Stanley T. Crooke, CRC Press, Boca Raton, Florida; and Sambrook et al., “Molecular Cloning, A laboratory Manual,” 2nd Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “excipient” means any compound or mixture of compounds that is added to a composition as provided herein that is suitable for delivery of an oligomeric compound.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety, phosphate-linked nucleosides also being referred to as “nucleotides”.

As used herein, “chemical modification” or “chemically modified” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

As used herein, “furanosyl” means a structure comprising a 5-membered ring comprising four carbon atoms and one oxygen atom.

As used herein, “naturally occurring sugar moiety” means a ribofuranosyl as found in naturally occurring RNA or a deoxyribofuranosyl as found in naturally occurring DNA. A “naturally occurring sugar moiety” as referred to herein is also termed as an “unmodified sugar moiety”. In particular, such a “naturally occurring sugar moiety” or an “unmodified sugar moiety” as referred to herein has a —H (DNA sugar moiety) or —OH (RNA sugar moiety) at the 2′-position of the sugar moiety, especially a —H (DNA sugar moiety) at the 2′-position of the sugar moiety.

As used herein, “sugar moiety” means a naturally occurring sugar moiety or a modified sugar moiety of a nucleoside. As used herein, “modified sugar moiety” means a substituted sugar moiety or a sugar surrogate.

As used herein, “substituted sugar moiety” means a furanosyl that has been substituted. Substituted sugar moieties include, but are not limited to furanosyls comprising substituents at the 2′-position, the 3′-position, the 5′-position and/or the 4′-position. Certain substituted sugar moieties are bicyclic sugar moieties.

As used herein, “2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring).

As used herein, “MOE” means —OCH2CH2OCH3.

As used herein, “2′-F nucleoside” refers to a nucleoside comprising a sugar comprising fluorine at the 2′ position. Unless otherwise indicated, the fluorine in a 2′-F nucleoside is in the ribo position (replacing the OH of a natural ribose). Duplexes of uniformly modified 2′-fluorinated (ribo) oligonucleotides hybridized to RNA strands are not RNase H substrates while the ara analogs retain RNase H activity.

As used herein the term “sugar surrogate” means a structure that does not comprise a furanosyl and that is capable of replacing the naturally occurring sugar moiety of a nucleoside, such that the resulting nucleoside sub-units are capable of linking together and/or linking to other nucleosides to form an oligomeric compound which is capable of hybridizing to a complementary oligomeric compound. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

As used herein, “nucleotide” means a nucleoside further comprising a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e. no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding, more specifically hydrogen bonding, with a complementary naturally occurring nucleobase of another oligonucleotide or nucleic acid. Nucleobases may be naturally occurring or may be modified.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides can comprise a modified sugar moiety and/or a modified nucleobase.

As used herein, “bicyclic nucleoside” or “BNA” means a nucleoside comprising a bicyclic sugar moiety.

As used herein, “locked nucleic acid nucleoside” or “LNA” means a nucleoside comprising a bicyclic sugar moiety comprising a 4′-CH2-O-2′bridge.

As used herein, “2 ‘-substituted nucleoside” means a nucleoside comprising a substituent at the 2’-position of the sugar moiety other than H or OH. Unless otherwise indicated, a 2′-substituted nucleoside is not a bicyclic nucleoside.

As used herein, “deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides.

As used herein, “modified oligonucleotide” means an oligonucleotide comprising at least one modified nucleoside and/or at least one modified internucleoside linkage.

As used herein, “linkage” or “linking group” means a group of atoms that link together two or more other groups of atoms.

As used herein “internucleoside linkage” means a covalent linkage between adjacent nucleosides in an oligonucleotide.

As used herein “naturally occurring internucleoside linkage” means a 3′ to 5′ phosphodiester linkage. As used herein, “modified internucleoside linkage” means any internucleoside linkage other than a naturally occurring internucleoside linkage. In particular, a “modified internucleoside linkage” as referred to herein can include a modified phosphorous linking group such as a phosphorothioate or phosphorodithioate internucleoside linkage.

As used herein, “terminal internucleoside linkage” means the linkage between the last two nucleosides of an oligonucleotide or defined region thereof.

As used herein, “phosphorus linking group” means a linking group comprising a phosphorus atom and can include naturally occurring phosphorous linking groups as present in naturally occurring RNA or DNA, such as phosphodiester linking groups, or modified phosphorous linking groups that are not generally present in naturally occurring RNA or DNA, such as phosphorothioate or phosphorodithioate linking groups. Phosphorus linking groups can therefore include without limitation, phosphodiester, phosphorothioate, phosphorodithioate, phosphonate, methylphosphonate, phosphoramidate, phosphorothioamidate, thionoalkylphosphonate, phosphotriesters, thionoalkylphosphotriester and boranophosphate.

As used herein, “internucleoside phosphorus linking group” means a phosphorus linking group that directly links two nucleosides.

As used herein, “oligomeric compound” means a polymeric structure comprising two or more substructures. In certain embodiments, an oligomeric compound comprises an oligonucleotide, such as a modified oligonucleotide. In certain embodiments, an oligomeric compound further comprises one or more conjugate groups and/or terminal groups and/or ligands. In certain embodiments, an oligomeric compound consists of an oligonucleotide. In certain embodiments, an oligomeric compound comprises a backbone of one or more linked monomeric sugar moieties, where each linked monomeric sugar moiety is directly or indirectly attached to a heterocyclic base moiety. In certain embodiments, oligomeric compounds may also include monomeric sugar moieties that are not linked to a heterocyclic base moiety, thereby providing abasic sites. Oligomeric compounds may be defined in terms of a nucleobase sequence only, i.e., by specifying the sequence of A, G, C, U (or T). In such a case, the structure of the sugar-phosphate backbone is not particularly limited and may or may not comprise modified sugars and/or modified phosphates. On the other hand, oligomeric compounds may be more comprehensively defined, i.e. by specifying not only the nucleobase sequence, but also the structure of the backbone, in particular the modification status of the sugars (unmodified, 2′-OMe modified, 2′-F modified etc.) and/or of the phosphates.

As used herein, “nucleic acid construct” or “construct” refers to an assembly of two or more, such as four oligomeric compounds. the oligomeric compounds may be connected to each other by covalent bonds such phosphodiester bonds as they occur in naturally occurring nucleic acids or modified versions thereof as disclosed herein, or by non-covalent bonds such as hydrogen bonds, optionally hydrogen bonds between nucleobases such as Watson-Crick base pairing. Optional is that a construct comprises four oligomeric compounds, two of which are connected covalently, thereby giving rise to two nucleic acid strands which nucleic acid strands are bound to each other by hydrogen bonds. Complementarity between the strand may be throughout, but is not necessarily so. In particular, optional embodiments provide for an antisense strand targeting TMPRSS6 to be connected covalently with a sense strand of an APOC3-targeting double stranded RNA molecule, and of the antisense strand of the APOC3-targeting double stranded RNA molecule to be connected covalently to a sense strand of a TMPRSS6-targeting double stranded RNA molecule. Since antisense and sense strands of the parent single-target-directed RNA molecules do not need to have the same length and optionally do not have the same length with antisense portions being longer than sense portions, an optional construct of the disclosure contains a central region where the 3′ regions of the antisense portions of the parent single-target-directed RNA molecules face each other. In that region generally no or only partial base pairing will occur, while full complementarity is not excluded. Otherwise, where antisense and sense portions of the respective parent RNA molecules face each other, there is complementarity, optionally full complementarity or 1 or 2 mismatches.

The term “strand” has its art-established meaning and refers to a plurality of linked nucleosides, the linker not being particularly limited, but including phosphodiesters and variants thereof as disclosed herein. A strand may also be viewed as a plurality of linked nucleotides in which case the linker would be a covalent bond.

As used herein, “terminal group” means one or more atom attached to either, or both, the 3′ end or the 5′ end of an oligonucleotide. In certain embodiments, a terminal group comprises one or more terminal group nucleosides.

As used herein, “conjugate” or “conjugate group” means an atom or group of atoms bound to an oligonucleotide or oligomeric compound. In certain embodiments, a conjugate group links a ligand to a modified oligonucleotide or oligomeric compound. In general, conjugate groups can modify one or more properties of the compound to which they are attached, including, but not limited to pharmacodynamic, pharmacokinetic, binding, absorption, cellular distribution, cellular uptake, charge and/or clearance properties.

As used herein, “conjugate linker” or “linker” in the context of a conjugate group means a portion of a conjugate group comprising any atom or group of atoms and which covalently link an oligonucleotide to another portion of the conjugate group. In certain embodiments, the point of attachment on the oligomeric compound is the 3′-oxygen atom of the 3′-hydroxyl group of the 3′ terminal nucleoside of the oligonucleotide. In certain embodiments the point of attachment on the oligomeric compound is the 5′-oxygen atom of the 5′-hydroxyl group of the 5′ terminal nucleoside of the oligonucleotide. In certain embodiments, the bond for forming attachment to the oligomeric compound is a cleavable bond. In certain such embodiments, such cleavable bond constitutes all or part of a cleavable moiety.

In certain embodiments, conjugate groups comprise a cleavable moiety (e.g., a cleavable bond or cleavable nucleoside) and ligand portion that can comprise one or more ligands, such as a carbohydrate cluster portion, such as an N-Acetyl-Galactosamine, also referred to as “GalNAc”, cluster portion. In certain embodiments, the carbohydrate cluster portion is identified by the number and identity of the ligand. For example, in certain embodiments, the carbohydrate cluster portion comprises 2 GalNAc groups. For example, in certain embodiments, the carbohydrate cluster portion comprises 3 GalNAc groups and this is particularly optional. In certain embodiments, the carbohydrate cluster portion comprises 4 GalNAc groups. Such ligand portions are attached to an oligomeric compound via a cleavable moiety, such as a cleavable bond or cleavable nucleoside. The ligands can be arranged in a linear or branched configuration, such as a biantennary or triantennary configurations.

As used herein, “cleavable moiety” means a bond or group that is capable of being cleaved under physiological conditions. In certain embodiments, a cleavable moiety is cleaved inside a cell or sub-cellular compartments, such as an endosome or lysosome. In certain embodiments, a cleavable moiety is cleaved by endogenous enzymes, such as nucleases. In certain embodiments, a cleavable moiety comprises a group of atoms having one, two, three, four, or more than four cleavable bonds. In certain embodiments, a cleavable moiety is a phosphodiester linkage. The ligands can be arranged in a linear or branched configuration, such as a biantennary or triantennary configurations. An optional carbohydrate cluster has the following formula:

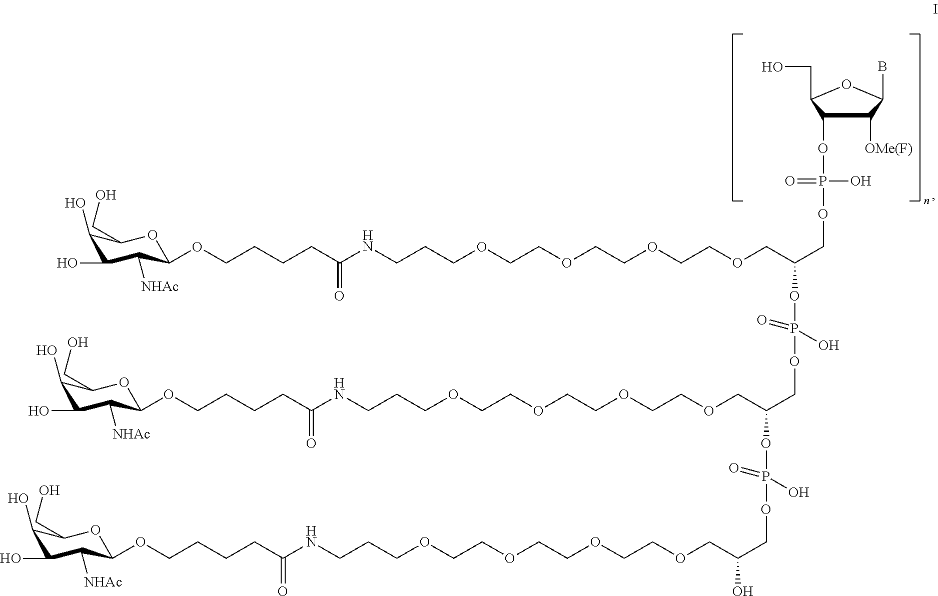

wherein in the structural formula one, two, or three phosphodiester linkages can also be substituted by phosphorothioate linkages.

As used herein, “cleavable moiety” means a bond or group that is cleaved under physiological conditions. In certain embodiments, a cleavable moiety is cleaved inside a cell or sub-cellular compartments, such as an endosome or lysosome. In certain embodiments, a cleavable moiety is cleaved by endogenous enzymes, such as nucleases. In certain embodiments, a cleavable moiety comprises a group of atoms having one, two, three, four, or more than four cleavable bonds. In certain embodiments, a cleavable moiety is a phosphodiester linkage.

As used herein, “cleavable bond” means any chemical bond capable of being broken.

As used herein, “carbohydrate cluster” means a compound having one or more carbohydrate residues attached to a linker group.

As used herein, “modified carbohydrate” means any carbohydrate having one or more chemical modifications relative to naturally occurring carbohydrates.

As used herein, “carbohydrate derivative” means any compound which may be synthesized using a carbohydrate as a starting material or intermediate.

As used herein, “carbohydrate” means a naturally occurring carbohydrate, a modified carbohydrate, or a carbohydrate derivative. A carbohydrate is a biomolecule including carbon (C), hydrogen (H) and oxygen (O) atoms. Carbohydrates can include monosaccharide, disaccharides, trisaccharides, tetrasaccharides, oligosaccharides or polysaccharides, such as one or more galactose moieties, one or more lactose moieties, one or more N-Acetyl-Galactosamine moieties, and/or one or more mannose moieties. A particularly optional carbohydrate is N-Acetyl-Galactosamine.

As used herein, “strand” means an oligomeric compound comprising linked nucleosides.

As used herein, “single strand” or “single-stranded” means an oligomeric compound comprising linked nucleosides that are connected in a continuous sequence without a break therebetween. Such single strands may include regions of sufficient self-complementarity so as to be capable of forming a stable self-duplex in a hairpin structure.

As used herein, “hairpin” means a single stranded oligomeric compound that includes a duplex formed by base pairing between sequences in the strand that are self-complementary and opposite in directionality.

As used herein, “hairpin loop” means an unpaired loop of linked nucleosides in a hairpin that is created as a result of hybridization of the self-complementary sequences. The resulting structure looks like a loop or a U-shape.

As used herein, “directionality” means the end-to-end chemical orientation of an oligonucleotide based on the chemical convention of numbering of carbon atoms in the sugar moiety meaning that there will be a 5′-end defined by the 5′ carbon of the sugar moiety, and a 3′-end defined by the 3′ carbon of the sugar moiety. In a duplex or double stranded oligonucleotide, the respective strands run in opposite 5′ to 3′ directions to permit base pairing between them.

As used herein, “duplex” means two or more complementary strand regions, or strands, of an oligonucleotide or oligonucleotides, hybridized together by way of non-covalent, sequence-specific interaction therebetween. Most commonly, the hybridization in the duplex will be between nucleobases adenine (A) and thymine (T), and/or (A) adenine and uracil (U), and/or guanine (G) and cytosine (C). The duplex may be part of a single stranded structure, wherein self-complementarity leads to hybridization, or as a result of hybridization between respective strands in a double stranded construct. It is optional that no nick occurs within a duplex.

As used herein, “double strand” or “double stranded” means a pair of oligomeric compounds that are hybridized to one another. In certain embodiments, a double-stranded oligomeric compound comprises a first and a second oligomeric compound.

As used herein, “expression” means the process by which a gene ultimately results in a protein. Expression includes, but is not limited to, transcription, post-transcriptional modification (e.g., splicing, polyadenlyation, addition of 5′-cap), and translation.

As used herein, “transcription” or “transcribed” refers to the first of several steps of DNA based gene expression in which a target sequence of DNA is copied into RNA (especially mRNA) by the enzyme RNA polymerase. During transcription, a DNA sequence is read by an RNA polymerase, which produces a complementary, antiparallel RNA sequence called a primary transcript.

As used herein, “target sequence” means a sequence to which an oligomeric compound is intended to hybridize to result in a desired activity with respect to TMPRSS6 and/or APOC3 expression. Oligonucleotides have sufficient complementarity to their target sequences to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In both DNA and RNA, guanine (G) is complementary to cytosine (C). In certain embodiments, complementary nucleobase means a nucleobase of an oligomeric compound that is capable of base pairing with a nucleobase of its target sequence. For example, if a nucleobase at a certain position of an oligomeric compound is capable of hydrogen bonding with a nucleobase at a certain position of a target sequence, then the position of hydrogen bonding between the oligomeric compound and the target sequence is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides) means the capacity of such oligomeric compounds or regions thereof to hybridize to a target sequence, or to a region of the oligomeric compound itself, through nucleobase complementarity.

Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or regions are 80%>complementary. In certain embodiments, complementary oligomeric compounds or regions are 90%>complementary. In certain embodiments, complementary oligomeric compounds or regions are at least 95% complementary. In certain embodiments, complementary oligomeric compounds or regions are 100% complementary.

As used herein, “self-complementarity” in reference to oligomeric compounds means a compound that may fold back on itself, creating a duplex as a result of nucleobase hybridization of internal complementary strand regions. Depending on how close together and/or how long the strand regions are, then the compound may form hairpin loops, junctions, bulges or internal loops.

As used herein, “mismatch” means a nucleobase of an oligomeric compound that is not capable of pairing with a nucleobase at a corresponding position of a target sequence, or at a corresponding position of the oligomeric compound itself when the oligomeric compound hybridizes as a result of self-complementarity, when the oligomeric compound and the target sequence and/or self-complementary regions of the oligomeric compound, are aligned.

As used herein, “hybridization” means the pairing of complementary oligomeric compounds (e.g., an oligomeric compound and its target sequence). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site.

As used herein, “fully complementary” in reference to an oligomeric compound or region thereof means that each nucleobase of the oligomeric compound or region thereof is capable of pairing with a nucleobase of a complementary nucleic acid target sequence or a self-complementary region of the oligomeric compound. Thus, a fully complementary oligomeric compound or region thereof comprises no mismatches or unhybridized nucleobases with respect to its target sequence or a self-complementary region of the oligomeric compound.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified naturally occurring RNA nucleoside are “differently modified,” even though the naturally occurring nucleoside is unmodified. Likewise, DNA and RNA oligonucleotides are “differently modified,” even though both are naturally-occurring unmodified nucleosides. Nucleosides that are the same but for comprising different nucleobases are not differently modified. For example, a nucleoside comprising a 2′-OMe modified sugar moiety and an unmodified adenine nucleobase and a nucleoside comprising a 2′-OMe modified sugar moiety and an unmodified thymine nucleobase are not differently modified.

As used herein, “the same type of modifications” refers to modifications that are the same as one another, including absence of modifications. Thus, for example, two unmodified RNA nucleosides have “the same type of modification,” even though the RNA nucleosides are unmodified. Such nucleosides having the same type modification may comprise different nucleobases.

As used herein, “region” or “regions”, or “portion” or “portions”, mean a plurality of linked nucleosides that have a function or character as defined herein, in particular with reference to the claims and definitions as provided herein. Typically such regions or portions comprise at least 10, at least 11, at least 12 or at least 13 linked nucleosides. For example, such regions can comprise 13 to 20 linked nucleosides, such as 13 to 16 or 18 to 20 linked nucleosides. Typically a first region as defined herein consists essentially of 18 to 20 nucleosides and a second region as defined herein consists essentially of 13 to 16 linked nucleosides.

As used herein, “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

As used herein, “substituent” and “substituent group,” means an atom or group that replaces the atom or group of a named parent compound. For example a substituent of a modified nucleoside is any atom or group that differs from the atom or group found in a naturally occurring nucleoside (e.g., a modified 2′-substituent is any atom or group at the 2 ‘-position of a nucleoside other than H or OH). Substituent groups can be protected or unprotected. In certain embodiments, compounds of the present disclosure have substituents at one or at more than one position of the parent compound. Substituents may also be further substituted with other substituent groups and may be attached directly or via a linking group such as oxygen or an alkyl or hydrocarbyl group to a parent compound.

Such substituents can be present as the modification on the sugar moiety, in particular a substituent present at the 2′-position of the sugar moiety. Unless otherwise indicated, groups amenable for use as substituents include without limitation, one or more of halo, hydroxyl, alkyl, alkenyl, alkynyl, acyl, carboxyl, alkoxy, alkoxyalkylene and amino substituents. Certain substituents as described herein can represent modifications directly attached to a ring of a sugar moiety (such as a halo, such as fluoro, directly attached to a sugar ring), or a modification indirectly linked to a ring of a sugar moiety by way of an oxygen linking atom that itself is directly linked to the sugar moiety (such as an alkoxyalkylene, such as methoxyethylene, linked to an oxygen atom, overall providing an MOE substituent as described herein attached to the 2′-position of the sugar moiety).

As used herein, “alkyl,” as used herein, means a saturated straight or branched monovalent C1-6 hydrocarbon radical, with methyl being a most optional alkyl as a substituent at the 2′-position of the sugar moiety. The alkyl group typically attaches to an oxygen linking atom at the 2′poisition of the sugar, therefore, overall providing a-Oalkyl substituent, such as an-OCH3 substituent, on a sugar moiety of an oligomeric compound according to the present disclosure. This will be well understood be a person skilled in the art.

As used herein, “alkylene” means a saturated straight or branched divalent hydrocarbon radical of the general formula —CnH2n- where n is 1-6. Methylene or ethylene are optional alkylenes.

As used herein, “alkenyl” means a straight or branched unsaturated monovalent C2-6 hydrocarbon radical, with ethenyl or propenyl being most optional alkenyls as a substituent at the 2′-position of the sugar moiety. As will be well understood in the art, the degree of unsaturation that is present in an alkenyl radical is the presence of at least one carbon to carbon double bond. The alkenyl group typically attaches to an oxygen linking atom at the 2′-position of the sugar, therefore, overall providing a —Oalkenyl substituent, such as an-OCH2CH═CH2 substituent, on a sugar moiety of an oligomeric compound according to the present disclosure. This will be well understood be a person skilled in the art.

As used herein, “alkynyl” means a straight or branched unsaturated C2-6 hydrocarbon radical, with ethynyl being a most optional alkynyl as a substituent at the 2′-position of the sugar moiety. As will be well understood in the art, the degree of unsaturation that is present in an alkynyl radical is the presence of at least one carbon to carbon triple bond. The alkynyl group typically attaches to an oxygen linking atom at the 2′-position of the sugar, therefore, overall providing a-Oalkynyl substituent on a sugar moiety of an oligomeric compound according to the present disclosure. This will be well understood be a person skilled in the art.

As used herein, “carboxyl” is a radical having a general formula —CO2H.

As used herein, “acyl” means a radical formed by removal of a hydroxyl group from a carboxyl radical as defined herein and has the general Formula —C(O)—X where X is typically C1-6 alkyl.

As used herein, “alkoxy” means a radical formed between an alkyl group, such as a C1-6 alkyl group, and an oxygen atom wherein the oxygen atom is used to attach the alkoxy group either to a parent molecule (such as at the 2′-position of a sugar moiety), or to another group such as an alkylene group as defined herein. Examples of alkoxy groups include without limitation, methoxy, ethoxy, propoxy, isopropoxy, n-butoxy, sec-butoxy and tert-butoxy. Alkoxy groups as used herein may optionally include further substituent groups.

As used herein, alkoxyalkylene means an alkoxy group as defined herein that is attached to an alkylene group also as defined herein, and wherein the oxygen atom of the alkoxy group attaches to the alkylene group and the alkylene attaches to a parent molecule. The alkylene group typically attaches to an oxygen linking atom at the 2′-position of the sugar, therefore, overall providing a —Oalkylenealkoxy substituent, such as an —OCH2CH2OCH3 substituent, on a sugar moiety of an oligomeric compound according to the present disclosure. This will be well understood by a person skilled in the art and is generally referred to as an MOE substituent as defined herein and as known in the art.

As used herein, “amino” includes primary, secondary and tertiary amino groups.

As used herein, “halo” and “halogen,” mean an atom selected from fluorine, chlorine, bromine and iodine.

As used herein, the term “muRNA” or “multi RNA” includes nucleic acid constructs comprising more than one, typically two, RNA sequences, i.e. first and second nucleic acid portion, the first nucleic acid portion targeting a region of TMPRSS6 mRNA and the second nucleic acid portion targeting a region of APOC3. The targeting RNA sequences are also referred to as “antisense” or “guide” strands, while the respective passenger strands, i.e. third and fourth nucleic acid portions being complementary to the first and second nucleic acid portions, respectively, are also included in the nucleic acid construct. In particular, such muRNA are designed such that subsequent to in vivo administration, they are disassembled and the first and second antisense sequences are released. A particular example for such muRNA is shown below, where (1) is the first nucleic acid portion, (2) is the third nucleic acid portion being complementary to (1), (3) is the second nucleic acid portion being complementary to the fourth nucleic acid portion, while (5) is a labile linker while (6) is a ligand, which will both be explained below.

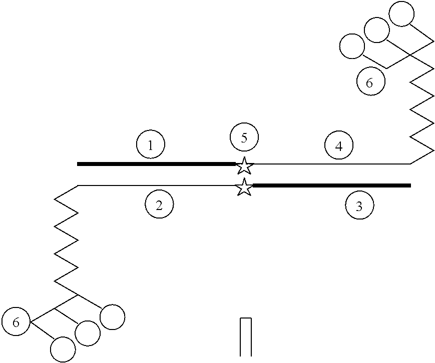

Further miniaturization by shortening the sense regions leads to bulge in the central part of the molecule where the 3′-terminal regions of the two antisense regions face each other:

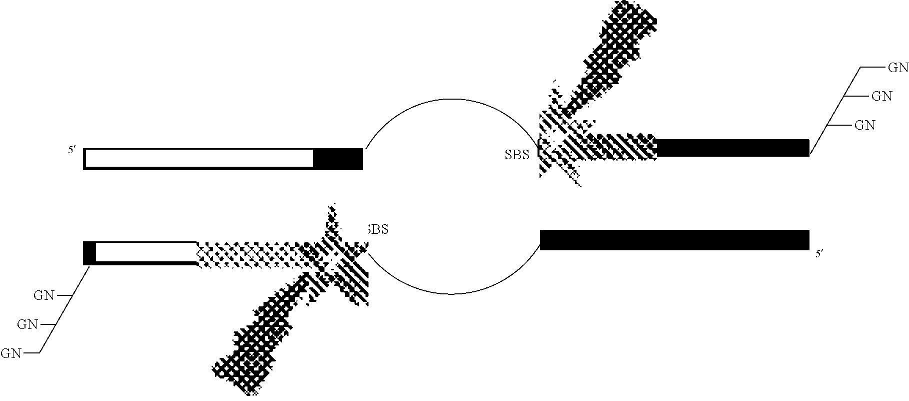

In the diagram above, “GN” designates a GalNAc moiety, and “SBS” designates the fragile site (“SBS”=Sollbruchstelle”) which may be implemented as a nucleoside with a non-modified sugar.

It will also be understood that oligomeric compounds as described herein may have one or more non-hybridizing nucleosides at one or both ends of one or both strands (overhangs) and/or one or more internal non-hybridizing nucleosides (mismatches) provided there is sufficient complementarity to maintain hybridization under physiologically relevant conditions. Alternatively, oligomeric compounds as described herein may be blunt ended at least one end.

The term “comprising” is used herein to mean including the method steps or elements identified, but that such steps or elements do not comprise an exclusive list and as such there may be present additional steps or elements.

Further, to the extent that the term “includes” is used in either the detailed description or the claims, such term is intended to be inclusive in a manner similar to the term “comprising” as “comprising” is interpreted when employed as a transitional word in a claim.

### The Present Disclosure Relates to the Following Embodiments

muRNA Nucleic Acid Constructs

According to a first aspect, the present disclosure is directed to a nucleic acid construct comprising at least:
(a) a first nucleic acid portion that is at least partially complementary to at least a first portion of an RNA which is transcribed from a TMPRSS6 gene;
(b) a second nucleic acid portion that is at least partially complementary to at least a second portion of an RNA which is transcribed from a APOC3 gene;
(c) a third nucleic acid portion that is at least partially complementary to the first nucleic acid portion of (a), so as to form a first nucleic acid duplex region therewith;
(d) a fourth nucleic acid portion that is at least partially complementary to the second nucleic acid portion of (b), so as to form a second nucleic acid duplex region therewith.

The first/second/third and fourth nucleic acid portions refer in their broadest sense to nucleobase sequences. In their narrower sense it is clear that these sequences may be composed of linked nucleosides or nucleotides. Complementarity is defined to allow for 0, 1, 2 or 3 mismatches between an antisense sequence and a target region, whereas all other nucleobases are complementary to the target region.

The construct may be designed such that subsequent to in vivo administration the construct disassembles to yield at least first and second discrete nucleic acid targeting molecules that respectively target the RNA portions transcribed from the target genes of (a) and (b);

whereby (i) the first nucleic acid targeting molecule is capable of modulating expression of the target gene of (a), and comprises, or is derived from, at least the first nucleic acid portion of (a), and (ii) the second nucleic acid targeting molecule is capable of modulating expression of the target gene of (b), and comprises, or is derived from, the second nucleic acid portion of (b).

The construct may be designed to disassemble such that the first and second discrete nucleic acid targeting molecules are respectively processed by independent RNAi-induced silencing complexes.

### Sequence Features, Labile Functionality and Structural Features of the RNA Molecules

The construct according to the first aspect and its aforementioned embodiments may comprise at least one labile functionality such that subsequent to in vivo administration the construct is cleaved so as to yield the at least first and second discrete nucleic acid targeting molecules.

The labile functionality may comprise one or more unmodified nucleotides, wherein optionally the one or more unmodified nucleotides of the labile functionality represent one or more cleavage positions within the construct whereby subsequent to in vivo administration the construct is cleaved at the one or more cleavage positions so as to yield the at least first and second discrete nucleic acid targeting molecules. Especially the cleavage positions are respectively located within the construct so that subsequent to cleavage the first discrete nucleic acid targeting molecule comprises, or is derived from, the first nucleic acid duplex region, and the second discrete nucleic acid targeting molecule comprises, or is derived from, the second nucleic acid duplex region Optionally the first discrete nucleic acid targeting molecule comprises or consists of the first nucleic acid portion of (a) and the third nucleic acid portion of (c), and/or the second discrete nucleic acid targeting molecule comprises or consists of the second nucleic acid portion of (b) and the fourth nucleic acid portion of (d).

### In Certain Embodiments,

(a) the first nucleic acid portion has a nucleobase sequence selected from SEQ ID NOs: 1 to 3 (see next paragraph);
(b) the second nucleic acid portion has a nucleobase sequence selected from Table 1 (SEQ ID NOs: 8 to 14) or SEQ ID NO: 29;
(c) the third nucleic acid portion has a nucleobase sequence selected from SEQ ID NOs: 15 to 17 (see next paragraph); and/or
(d) the fourth nucleic acid portion has a nucleobase sequence selected from Table 2 (SEQ ID NOs: 22 to 28) or SEQ ID NO: 30.

First nucleic acid portion sequences (19 mers):
SEQ ID No. 1 (TMPf, antisense):
UGUACCCUAGGAAAUACCA
SEQ ID No. 2 (X311, antisense):
UUGUACCCUAGGAAAUACC
SEQ ID No. 3 (X312, antisense):
AACCAGAAGAAGCAGGUGA
Third nucleic acid portion sequences (15 mers):
SEQ ID No. 15 (TMPf, sense):
AUUUCCUAGGGUACA
SEQ ID No. 16 (X311, sense):
UUUCUUAGGGUACAA
SEQ ID No. 17 (X312, sense):
CUGCUUCUUCUGGUU

In certain embodiments, the first nucleic acid portion of (a) is directly or indirectly linked to the fourth nucleic acid portion of (d) as a primary structure.

In certain embodiments, the first and the fourth nucleic acid portions have the nucleobase sequences of SEQ ID NOs: 1 and 24; 1 and 22; 1 and 25; 1 and 26; 1 and 28; 1 and 30; 3 and 24; 3 and 22; 3 and 25; 3 and 26; 3 and 28; 3 and 30; 2 and 24; 2 and 22; 2 and 25; 2 and 26; 2 and 28; 2 and 30, respectively, optionally 1 and 24.

In certain embodiments, the second nucleic acid portion of (b) is directly or indirectly linked to the third nucleic acid portion of (c) as a primary structure.

In certain embodiments, the second and third nucleic acid portions have the nucleobase sequences of SEQ ID NOs: 10 and 15; 8 and 15; 11 and 15; 12 and 15; 14 and 15; 29 and 15; 10 and 16; 8 and 16; 11 and 16; 12 and 16; 14 and 16; 29 and 16; 10 and 17; 8 and 17; 11 and 17; 12 and 17; 14 and 17; 29 and 17, respectively, optionally 10 and 15.

In certain embodiments, the nucleic acid construct further comprises 1 to 8 additional nucleic acid portions that are respectively at least partially complementary to an additional 1 to 8 portions of RNA transcribed from one or more target genes, which target genes may be the same or different to each other, and/or the same or different to the target genes defined in (a) and/or (b), and wherein each of the 1 to 8 additional nucleic acid portions respectively form additional duplex regions with respective passenger nucleic acid portions that are respectively at least partially complementary therewith.

In certain embodiments, the second nucleic acid portion of (b), and the 1 to 8 additional nucleic acid portions, are directly or indirectly linked to selected passenger nucleic acid portions as respective primary structures.

In certain embodiments, the direct or indirect linking represents either (i) an internucleotide bond, (ii) an internucleotide nick, or (iii) a nucleic acid linker portion of 1, 2, 3, 4, 5, 6, 7, 8, 9, or 10 nucleotides, the nucleic acid linker optionally being single stranded.

In certain embodiments, the linking is direct, thereby giving rise to (a) contiguous strand(s).

In certain embodiments, there exists some complementarity between the first nucleic acid portion of (a) and the second nucleic acid portion of (b), or the third nucleic acid portion of (c) and the fourth nucleic acid portion of (d).

In certain embodiments, the complementarity
(i) is/are 1, 2, 3, 4, 5, 6, 7, 8, 9, or 10, optionally 2, 3, 4 or 5 base pairs; and/or
(ii) is between the first nucleic acid portion of (a) and the second nucleic acid portion of (b).

In certain embodiments, wherein the internucleotide bond involves at least one of the one or more unmodified nucleotides, wherein optionally cleavage occurs at the 3′ position of (at least one of) the unmodified nucleotide(s).

In certain embodiments, the first nucleic acid portion of (a), and/or the second nucleic acid portion of (b), and/or the third nucleic acid portion of (c), and/or the fourth nucleic acid portion of (d), are respectively 7 to 25 nucleotides in length.

In certain embodiments, the first nucleic acid portion of (a) and/or the second nucleic acid portion of (b) have a length of 18 to 21, more optionally 18 to 20, and yet more optionally 19 nucleotides.

In certain embodiments, the third nucleic acid portion of (c), and/or the fourth nucleic acid portion of (d) have a length of 11 to 20, more optionally 13 to 16, and yet more optionally 14 or 15, most optionally 14 nucleotides.

In certain embodiments, the unmodified nucleotide(s) is/are at any of position 18 to 25, more optionally at any of positions 18 to 21, and/or the 3′ terminal position of the first nucleic acid portion of (a) and/or of the third nucleic acid portion of (c).

In certain embodiments, the unmodified nucleotide is at position 19.

In certain embodiments, wherein the nucleic acid linker portion is 1 to 8 nucleotides in length, optionally 2 to 7 or 3 to 6 nucleotides in length, more optionally about 4 or 5 and most optionally 4 nucleotides in length.

In certain embodiments, one, more of all of the duplex regions independently have a length of 10 to 19, more optionally 13 to 19, and yet more optionally 13, 14 or 15 base pairs, most optionally 14 base pairs, wherein optionally there is one mismatch within the duplex region.

### In Certain Embodiments,

(a) the first nucleic acid portion is selected from Table 3a;
(b) the second nucleic acid portion is selected from Table 3b;
(c) the third nucleic acid portion is selected from Table 4a; and/or
(d) the fourth nucleic acid portion is selected from Table 4b.

In certain embodiments, the muRNA construct comprises two strands, wherein the first strand is selected from Table 5a and the second strand from Table 5b. Alternatively, the first and second strands are selected from Table 7a, wherein in particular the first and second strands are jointly selected from SEQ ID NO: 634, 635, 636, 637, 638, 639, 640, 641, 642, and 643. Optionally, the muRNA constructs are consisting of the group selected from the combinations (two strands constituting a muRNA) of SEQ ID NOs: 634+635, 636+637, 638+639, 640+641 and 642+643.

In other words, the first strand may be
(SEQ ID NO. 634)
[5Phos][mU][Ps][fG][Ps][mG][fA][mU][fU][mU][fG][mG][fA][mG][fA][mA][fU][mG][Ps][fA]
[Ps][mA][Ps][fC][Ps][rC][fG][Ps][mG][Ps][fU][mA][fC][mU][fC][mC][fU][mU][fG][mU][fU]
[Ps][mG][Ps][fA][Ps][3XGalNAc]
and
the second strand may be
(SEQ ID NO. 635)
[5Phos][mU][Ps][fC][Ps][mA][fA][mC][fA][mA][fG][mG][fA][mG][fU][mA][fC][mC][Ps][fC]
[Ps][mG][Ps][fG][Ps][rG][fC][Ps][mA][Ps][fU][mU][fC][mU][fC][mC][fA][mA][fA][mU][fC]
[Ps][mC][Ps][fA][Ps][3xGalNAc];
or
the first strand may be
(SEQ ID NO. 636)
[5Phos][mU][Ps][fA][Ps][mA][fA][mG][fG][mG][fC][mA][fG][mC][fU][mG][fA][mG][Ps][fC]
[Ps][mU][Ps][fC][Ps][rA][fG][Ps][mG][Ps][fU][mA][fC][mU][fC][mC][fU][mU][fG][mU][fU][
Ps][mG][Ps][fA][Ps][3XGalNAc]
and
the second strand may be
(SEQ ID NO. 637)
[5Phos][mU][Ps][fC][Ps][mA][fA][mC][fA][mA][fG][mG][fA][mG][fU][mA][fC][mC][Ps][fC]
[Ps][mG][Ps][fG][Ps][rG][fC][Ps][mU][Ps][fC][mA][fG][mC][fU][mG][fC][mC][fC][mU][fU]
[Ps][mU][Ps][fA][Ps][3xGalNAc];
or
the first strand may be
(SEQ ID NO. 638)
[5Phos][mU][Ps][fA][Ps][mC][fG][mC][fA][mG][fU][mU][fU][mC][fU][mC][fU][mC][Ps][fA]
[Ps][mU][Ps][fC][Ps][rC][fG][Ps][mG][Ps][fU][mA][fC][mU][fC][mC][fU][mU][fG][mU][fU]
[Ps][mG][Ps][fA][Ps][3XGalNAc]
and
the second strand may be
(SEQ ID NO. 639)
[5phos][mU][Ps][fC][Ps][mA][fA][mC][fA][mA][fG][mG][fA][mG][fU][mA][fC][mC][Ps][fC][
Ps][mG][Ps][fG][Ps][rG][fG][Ps][mA][Ps][fG][mA][fG][mA][fA][mA][fC][mU][fG][mC][fG][
Ps][mU][Ps][fA][Ps][3xGalNAc];
or
the first strand may be:
(SEQ ID NO. 640)
[5Phos][mU][Ps][fG][Ps][mC][fA][mG][fC][mU][fU][mU][fA][mU][fU][mC][fC][mA][Ps][fA]
[Ps][mA][Ps][fG][Ps][rG][fG][Ps][mG][Ps][fU][mA][fC][mU][fC][mC][fU][mU][fG][mU][fU]
[Ps][mG][Ps][fA][Ps][3XGalNAc]
and
the second strand may be
(SEQ ID NO. 641)
[Phos][mU][Ps][fC][Ps][mA][fA][mC][fA][mA][fG][mG][fA][mG][fU][mA][fC][mC][Ps][fC][
Ps][mG][Ps][fG][Ps][rG][fU][Ps][mG][Ps][fG][mA][fA][mU][fA][mA][fA][mG][fC][mU][fG]
[Ps][mC][Ps][fA][Ps][3xGalNAc];
or
the first strand may be
(SEQ ID NO. 642)
[5Phos][mU][Ps][fC][Ps][mA][fG][mU][fU][mU][fC][mU][fC][mU][fC][mA][fU][mC][Ps][fC][
Ps][mA][Ps][fG][Ps][rG][fG][Ps][mG][Ps][fU][mA][fC][mU][fC][mC][fU][mU][fG][mU][fU][P
s][mG][Ps][fA][Ps][3XGalNAc]
and
the second strand may be
(SEQ ID NO. 643)
[5Phos][mU][Ps][fC][Ps][mA][fA][mC][fA][mA][fG][mG][fA][mG][fU][mA][fC][mC][Ps][fC][
Ps][mG][Ps][fG][Ps][rG][fG][Ps][mA][Ps][fU][mG][fA][mG][fA][mG][fA][mA][fA][mC][fU][
Ps][mG][Ps][fA][Ps][3xGalNAc].

Further alternatively, the first and second strands are selected from Table 7b, wherein in particular the first and second strands are jointly selected from SEQ ID NO: 644, 645, 646, 647, 648, 649, 650, 651, 652 and 653. Optionally, the muRNA constructs are consisting of the group selected from the combinations (two strands constituting a muRNA) of SEQ ID NOs: 634+635, 636+637, 638+639, 640+641 and 642+643.

In other words, the first strand may be
UGGAUUUGGAGAAUGAACCGGUACUCCUUGUUGA (SEQ ID NO. 644) and the second strand may be UCAACAAGGAGUACCCGGGCAUUCUCCAAAUCCA (SEQ ID NO. 645); or
the first strand may be UAAAGGGCAGCUGAGCUCAGGUACUCCUUGUUGA (SEQ ID NO. 646) and the second strand may be UCAACAAGGAGUACCCGGGCUCAGCUGCCCUUUA (SEQ ID NO. 647); or
the first strand may be UACGCAGUUUCUCUCAUCCGGUACUCCUUGUUGA (SEQ ID NO. 648) and the second strand may be UCAACAAGGAGUACCCGGGGAGAGAAACUGCGUA (SEQ ID NO. 649); or
the first strand may be: UGCAGCUUUAUUCCAAAGGGGUACUCCUUGUUGA (SEQ ID NO. 650)
and the second strand may be UCAACAAGGAGUACCCGGGUGGAAUAAAGCUGCA (SEQ ID NO. 651) or
the first strand may be UCAGUUUCUCUCAUCCAGGGGUACUCCUUGUUGA (SEQ ID NO. 652) and the second strand may be UCAACAAGGAGUACCCGGGGAUGAGAGAAACUGA (SEQ ID NO. 653).

In certain embodiments, first and second strands are as shown below:

(SEQ ID NO. 670)
[mU][#][fG][#][mU][fA][mC][fC][mC][fU][mA][fG][mG]
[fA][mA][fA][mU][#][fA][#][mC][#][fC][#][rA][mG]
[#][fU][#][mA][fC][mU][fC][mC][fU][mU][fG][mU]
[fU][#][mG][#][fA][#][3XGalNAc];
and
(SEQ ID NO. 672)
[mU][#][fC][#][mA][fA][mC][fA][mA][fG][mG][fA][mG]
[fU][mA][fC][#][mC][#][fC][#][mG][#][fG][#][rG]
[fA][#][mU][#][fU][mU][fC][mC][fU][mA][fG][mG]
[fG][mU][fA][#][mC][#][fA][#][3XGalNAc],

wherein [mN], N being any nucleoside, designates 2′-OMe; [fN], N being any nucleoside, designates: 2′-F; [rA], N being any nucleoside, designates: 2′-OH; [#] designates a phosphorothioate connecting two adjacent nucleosides; and [3XGalNAc] designates the following ligand, wherein the strand to which the ligand is bound is shown in square brackets:

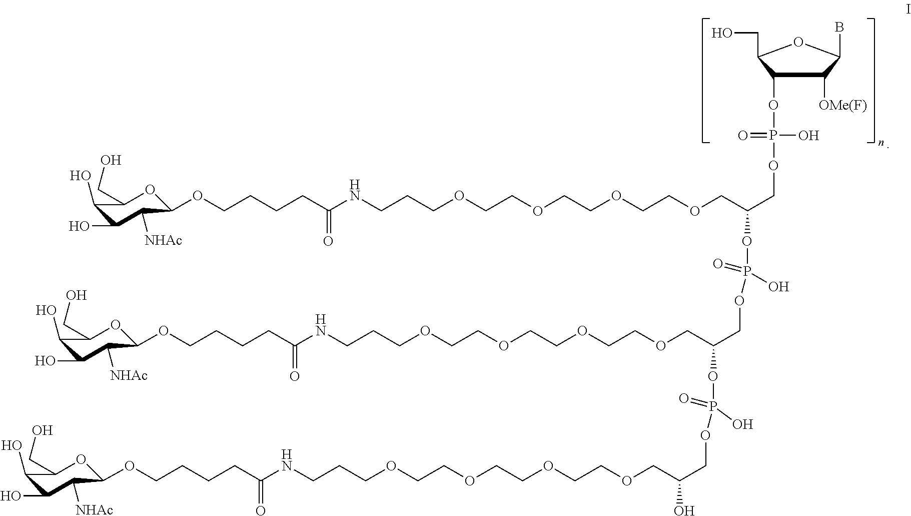

In certain embodiments, the 3′ terminal positions of the first and the third nucleic acid portions are replaced with an unmodified nucleotide.

### In Certain Embodiments,

(a) the first nucleic acid portion comprises at least 18, optionally 19, contiguous nucleotides allowing for up to three mismatches with a sequence being selected from Table 6a, wherein optionally the first antisense sequence is selected from SEQ ID NOs: 65, 127, 153, 185, and 203;
(b) the second nucleic acid portion comprises at least 18, optionally 19, contiguous nucleotides allowing for up to three mismatches with a sequence being selected from Table 1 (SEQ ID NOs: 8 to 14) or SEQ ID NO: 29;
(c) the third nucleic acid portion comprises at least 11, optionally 15, contiguous nucleotides allowing for up to three mismatches with a sequence being complementary to the first nucleic acid portion of (a), wherein optionally the first sense sequence is selected from 15 contiguous nucleotides of a sequence being complementary to a sequence selected from SEQ ID NOs 65, 127, 153, 185, and 203; and/or
(d) the fourth nucleic acid portion has a nucleobase sequence selected from Table 2 (SEQ ID NOs: 22 to 28) or SEQ ID NO: 30.

The third nucleic acid portion may alternatively be independently selected from Table 6b, such as from SEQ ID NOs 265, 327, 353, 385 and 406, wherein optionally at least 11, more optionally 15, contiguous nucleotides out of the sequence in Table 6b may constitute the first and/or the second sense sequence. More optionally, the first and/or the second sense sequence comprises or consists of the first 15 contiguous nucleotides from the corresponding one selected from Table 6b, such as from SEQ ID NOs 265, 327, 353, 385 and 406, counted from the 3′ terminus, wherein the last nucleotide at the 3′ terminus of the sequence carries an adenine “A” base replacing the base indicated in Table 6b.

Especially, the first and second antisense sequence have identical sequences being selected from SEQ ID NOs: 65, 127, 153, 185, and 203. The first and the second sense sequences may be selected complementary sequences of SEQ ID NOs: 65, 127, 153, 185, and 203, each of the complementary sequences comprising at least 15 contiguous nucleotides, wherein the last nucleotide at the 3′ terminus of the sequence comprising 15 contiguous nucleotides carries an adenine “A” base.

Since the present inventors surprisingly found in several instances that outstanding performance in single-targeting molecules (such as mxRNAs) may be transferred to double-targeting molecules (such as muRNAs), any further sequences, in particular antisense sequences as disclosed in the above-mentioned patent documents may serve as a basis for designing muRNAs of the present disclosure.

### In Certain Embodiments,

(a) the first nucleic acid portion is selected from Table 6c, in particular from SEQ ID NO: 465, 527, 553, 585, and 603;
(b) the second nucleic acid portion is selected from Table 3b;
(c) the third nucleic acid portion comprises at least 14, in particular 15, contiguous nucleotides being complementary to the corresponding part of the first nucleic acid portion; and/or
(d) the fourth nucleic acid portion is selected from Table 4b.

In certain embodiments, the 3′ terminal positions of the first antisense sequence is carries an unmodified nucleotide.

In certain embodiments, the first nucleic acid portion of (a) has a greater number of linked nucleosides compared to the third nucleic acid portion of (c), wherein optionally
a ratio between a total number of linked nucleosides of the first nucleic acid portion of (a) and a total number of linked nucleosides of the third nucleic acid portion of (c) ranges from about 19/16 to about 19/8, or from about 18/16 to about 18/8, wherein more optionally the ratio is 19/15 or 19/14, wherein the same may also apply for the second nucleic acid portion and the fourth nucleic acid portion.

In certain embodiments, the first antisense sequence of (a) has a greater number of linked nucleosides compared to the first sense sequence of (c), wherein optionally a percentage of the total number of the first antisense sequence of (a) relative to the total number of nucleosides of the entire first strand encompassing the first antisense sequence of (a) and the second sense sequence of (d) ranges from about to about 55% to about 60%, optionally from about 55% to about 56%, the same may apply to the second antisense sequence of (b) and the first sense sequence of (c).

In certain embodiment, the first nucleic acid portion is selected from Table 6a, in particular from SEQ ID NOs: 65, 127, 153, 185, and 203.

In certain embodiments, the third nucleic acid portion is selected from Table 6b and in particular has a length of 15 nucleotides counted from the 5′ end, wherein the sequence is in particular selected form SEQ ID NO: 265, 327, 353, 385, and 403.

In certain embodiments, first nucleic acid portion is selected from Table 6c, in particular from SEQ ID NO: 465, 527, 553, 585, and 603.

In certain embodiments, the third nucleic acid portion is selected from Table 6b and in particular has a length of 15 nucleotides counted from the 5′ end, wherein the sequence is in particular selected from SEQ ID NO: 265, 327, 353, 385, and 403.

### Ligands

The nucleic acid construct according to the first aspect and the aforementioned embodiments may further comprise one or more ligands.

In certain embodiments, the first nucleic acid portion of (a), and/or the second nucleic acid portion of (b), and/or the third nucleic acid portion of (c), and/or the fourth nucleic acid portion of (d), and/or, to the extent present, the 1 to 8 additional nucleic acid portions as defined previously herein, and/or the passenger nucleic acid portions as defined previously herein, respectively have a 5′ to 3′ directionality thereby defining 5′ and 3′ regions thereof.

In certain embodiments one or more ligands are conjugated at the 3′region, optionally the 3′ end, of any of (i) the third nucleic acid portion of (c), and/or (ii) the fourth nucleic acid portion of (d), and/or, to the extent present, the (iii) passenger nucleic acid portions as defined previously herein.

In certain embodiments, one or more ligands are conjugated at one or more regions intermediate of the 5′ and 3′ regions of any of the nucleic acid portions, optionally of the third nucleic acid portion of (c), and/or the fourth nucleic acid portion of (d), and/or the passenger nucleic acid portions as defined in claim 14 or 15.

In certain embodiments, one or more ligands are conjugated at the 5′ region, optionally the 5′ end, of any of the nucleic acid portions.

In certain embodiments, the one or more ligands are any cell directing moiety, such as lipids, carbohydrates, aptamers, vitamins and/or peptides that bind cellular membrane or a specific target on cellular surface. In an optional embodiment, the one or more carbohydrates can be a monosaccharide, disaccharide, trisaccharide, tetrasaccharide, oligosaccharide or polysaccharide In a more optional embodiment, the one or more carbohydrates comprise one or more hexose moieties. Especially the one or more hexose moieties are one or more galactose moieties, one or more lactose moieties, one or more N-Acetyl-Galactosamine moieties, and/or one or more mannose moieties. In particular, the hexose moiety may comprise two or three N-Acetyl-Galactosamine moieties.

In certain embodiments, the one or more ligands are attached in a linear configuration, or in a branched configuration. Optionally, the one or more ligands are attached as a biantennary or triantennary configuration, or as a configuration based on single ligands at different positions.

Optionally, the ligand has the following structure:

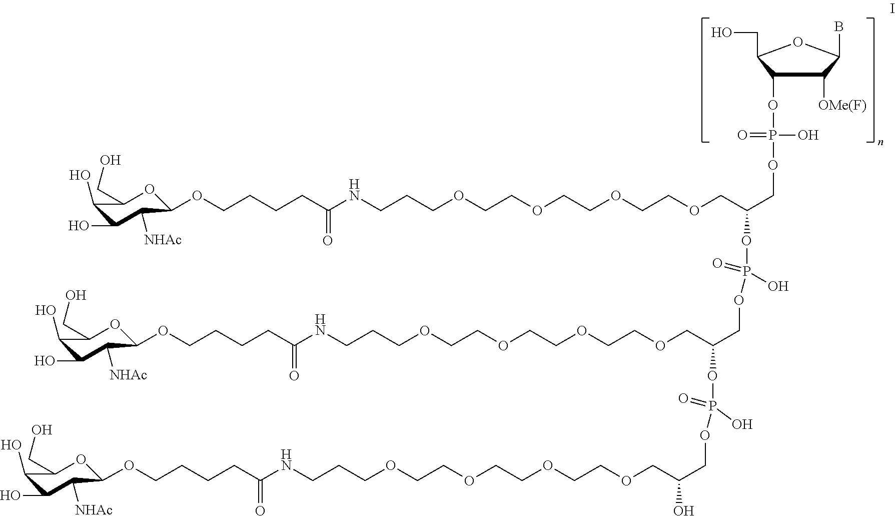

### Internucleoside Linkages

The nucleic acid construct according to the first aspect of the present disclosure or its aforementioned embodiments may comprise one or more phosphorothioate or phosphorodithioate internucleotide linkages.

In certain embodiments, the nucleic acid construct may comprise 1 to 15 phosphorothioate or phosphorodithioate internucleotide linkages.

In certain embodiments, the nucleic acid construct comprises one or more phosphorothioate or phosphorodithioate internucleotide linkages at one or more of the 5′ and/or 3′ regions of the first nucleic acid portion of (a), and/or the second nucleic acid portion of (b), and/or the third nucleic acid portion of (c), and/or the fourth nucleic acid portion of (d), and/or the 1 to 8 additional nucleic acid portions as defined previously herein, and/or the passenger nucleic acid portions as defined previously herein.

In certain embodiments, the nucleic acid construct comprises phosphorothioate or phosphorodithioate internucleotide linkages between at least two adjacent nucleotides of the nucleic acid linker portion as defined previously herein.

In certain embodiments, the nucleic acid construct comprises a phosphorothioate or phosphorodithioate internucleotide linkage between each adjacent nucleotide that is present in the nucleic acid linker portion.

In certain embodiments, the nucleic acid construct comprises a phosphorothioate or phosphorodithioate internucleotide linkage linking:
the first nucleic acid portion of (a) to the nucleic acid linker portion as defined previously herein; and/or
the second nucleic acid portion of (b) to the nucleic acid linker portion as defined previously herein; and/or
the third nucleic acid portion of (c) to the nucleic acid linker portion as defined previously herein and/or
the fourth nucleic acid portion of (d) to the nucleic acid linker portion as defined previously herein; and/or
the 1 to 8 additional nucleic acid portions as defined previously herein to the nucleic acid linker portion as defined previously herein; and/or
the passenger nucleic acid portions as defined previously herein to the nucleic acid linker portion as defined previously herein.

### Modifications

In the nucleic acid construct according to the first aspect of the present disclosure and its aforementioned embodiments, at least one nucleotide of at least one of the following is modified:
the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions as defined previously herein; and/or
to the extent present, the passenger nucleic acid portions as defined previously herein; and/or
to the extent present, the nucleic acid linker portion as previously herein.

In certain embodiments, one or more of the odd numbered nucleotides starting from the 5′ region of one of the following are modified, and/or wherein one or more of the even numbered nucleotides starting from the 5′ region of one of the following are modified, wherein typically the modification of the even numbered nucleotides is a second modification that is different from the modification of odd numbered nucleotides:
the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions as defined previously herein; and/or
to the extent present, the passenger nucleic acid portions as defined previously herein.

In certain embodiments, one or more of the odd numbered nucleotides starting from the 3′ region of the third nucleic acid portion of (c) are modified by a modification that is different from the modification of odd numbered nucleotides starting from the 5′ region of the first nucleic acid portion of (a); and/or
wherein one or more of the odd numbered nucleotides starting from the 3′ region of the fourth nucleic acid portion of (d) are modified by a modification that is different from the modification of odd numbered nucleotides starting from the 5′ region of the second nucleic acid portion of (b); and/or
wherein one or more of the odd numbered nucleotides starting from the 3′ region of the passenger nucleic acid portions as defined previously herein, to the extent present, are modified by a modification that is different from the modification of odd numbered nucleotides starting from the 5′ region of the 1 to 8 additional nucleic acid portions as defined previously herein; and/or
wherein one or more of the nucleotides of a nucleic acid linker portion as defined previously herein, to the extent present, are modified by a modification that (i) is different from the modification of an adjacent nucleotide of the 3′ region of the first nucleic acid portion of (a); and/or (ii) is different from the modification of an adjacent nucleotide of the 3′ region of the second nucleic acid portion of (b); and/or is different from the modification of an adjacent nucleotide of the 3′ region of the 1 to 8 additional nucleic acid portions, to the extent present, as defined previously herein.

In certain embodiments, one or more of the even numbered nucleotides starting from the 3′ region of: (i) the third nucleic acid portion of (c), and/or (ii) the fourth nucleic acid portion of (d), and/or (iii) the passenger nucleic acid portions as defined previously herein, to the extent present, are modified by a modification that is different from the modification of odd numbered nucleotides starting from the 3′ region of these respective portions.

In certain embodiments, at least one or more of the modified even numbered nucleotides of (i) the first nucleic acid portion of (a), and/or (ii) the second nucleic acid portion of (b), and/or (iii), to the extent present, the 1 to 8 additional nucleic acid portions as defined previously herein, is adjacent to at least one or more differently modified odd numbered nucleotides of these respective portions.

In certain embodiments, at least one or more of the modified even numbered nucleotides of (i) the third nucleic acid portion of (c), and/or (ii) the fourth nucleic acid portion of (d), and/or (iii), to the extent present, the passenger nucleic acid portions as defined previously herein, is adjacent to at least one or more differently modified odd numbered nucleotides of these respective portions.

In certain embodiments, a plurality of adjacent nucleotides of (i) the first nucleic acid portion of (a), and/or (ii) the second nucleic acid portion of (b), and/or (iii), to the extent present, the 1 to 8 additional nucleic acid portions as defined previously herein, are modified by a common modification.

In certain embodiments, a plurality of adjacent nucleotides of (i) the third nucleic acid portion of (c), and/or (ii) the fourth nucleic acid portion of (d), and/or (iii), to the extent present, the passenger nucleic acid portions as defined previously herein, are modified by a common modification.

In certain embodiments, the plurality of adjacent commonly modified nucleotides are 2 to 4 adjacent nucleotides, optionally 3 or 4 adjacent nucleotides.

In certain embodiments, the plurality of adjacent commonly modified nucleotides are located in the 5′ region of (i) the third nucleic acid portion of (c), and/or (ii) the fourth nucleic acid portion of (d), and/or (iii), to the extent present, the passenger nucleic acid portions as defined previously herein.

In certain embodiments, a plurality of adjacent commonly modified nucleotides are located in the nucleic acid linker portion as defined previously herein.

In certain embodiments, the one or more of the modified nucleotides of first nucleic acid portion of (a) do not have a common modification present in the corresponding nucleotide of the third nucleic acid portion of (c) of the first duplex region; and/or one or more of the modified nucleotides of second nucleic acid portion of (b) do not have a common modification present in the corresponding nucleotide of the fourth nucleic acid portion of (d) of the second duplex region; and/or one or more of the modified nucleotides of the 1 to 8 additional nucleic acid portions, to the extent present, as defined previously herein, do not have a common modification present in the corresponding nucleotide of the corresponding passenger nucleic acid portions of the respective duplex regions.

In certain embodiments, the one or more of the modified nucleotides of the first nucleic acid portion of (a) are shifted by at least one nucleotide relative to a commonly modified nucleotide of the third nucleic acid portion of (c); and/or one or more of the modified nucleotides of the second nucleic acid portion of (b) are shifted by at least one nucleotide relative to a commonly modified nucleotide of the fourth nucleic acid portion of (d); and/or one or more of the modified nucleotides of the 1 to 8 additional nucleic acid portions, to the extent present, as defined previously herein are shifted by at least one nucleotide relative to a commonly modified nucleotide of the passenger nucleic acid portions, to the extent present, as defined previously herein.

In certain embodiments, the modification and/or modifications are each and individually sugar, phosphate, or base modifications.

In certain embodiments, the modification is selected from nucleotides with 2′ modified sugars; conformationally restricted nucleotides (CRN) sugar such as locked nucleic acid (LNA), (S)-constrained ethyl bicyclic nucleic acid, and constrained ethyl (cEt), tricyclo-DNA;

morpholino, unlocked nucleic acid (UNA), glycol nucleic acid (GNA), D-hexitol nucleic acid (HNA), and cyclohexene nucleic acid (CeNA).

In certain embodiments, the 2′ modified sugar is selected from 2′-O-alkyl modified sugar, 2′-O-methyl modified sugar, 2′-O-methoxyethyl modified sugar, 2′-O-allyl modified sugar, 2′-C-allyl modified sugar, 2′-deoxy modified sugar such as 2′-deoxy ribose, 2′-F modified sugar, 2′-arabino-fluoro modified sugar, 2′-O-benzyl modified sugar, 2′-amino modified sugar, and 2′-O-methyl-4-pyridine modified sugar.

In certain embodiments, the base modification is any one of a an abasic nucleotide and a non-natural base comprising nucleotide.

In certain embodiments, at least one modification is a 2′-O-methyl modification in a ribose moiety.

In certain embodiments, at least one modification is a 2′-F modification in a ribose moiety.

In certain embodiments, the nucleotides at any of positions 2 and 14 downstream from the first nucleotide of the 5′ region of (i) the first nucleic acid portion of (a); and/or (ii) the second nucleic acid portion of (b); and/or (iii), to the extent present, the 1 to 8 additional nucleic acid portions as defined previously herein; do not contain 2′-O-methyl modifications in ribose moieties.

In certain embodiments, one, two or all three nucleotides of (i) the third nucleic acid portion of (c); and/or (ii) the fourth nucleic acid portion of (d); and/or (iii), to the extent present, the passenger nucleic acid portions as defined previously herein; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii) the 1 to 8 additional nucleic acid portions, to the extent present, as defined previously herein; do not contain 2′-O-methyl modifications in ribose moieties.

In certain embodiments, the nucleotides at any of positions 2 and 14 downstream from the first of (i) the first nucleic acid portion of (a); and/or (ii) the second nucleic acid portion of (b); and/or (iii), to the extent present, the 1 to 8 additional nucleic acid portions as defined previously herein; contain 2′-F modifications in ribose moieties.

In certain embodiments, one, two or all three nucleotides of (i) the third nucleic acid portion of (c); and or (ii) the fourth nucleic acid portion of (d); and/or (iii), to the extent present, the passenger nucleic acid portions as defined previously herein; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii), to the extent present, the 1 to 8 additional nucleic acid portions as defined previously herein; contain 2′-F modifications in ribose moieties.

In certain embodiment all remaining nucleotides contain either 2′-O-methyl modifications or 2′-F modifications in ribose moieties, optionally with the exception of the unmodified nucleotide(s) in accordance with an embodiment defined previously herein.

In certain embodiments, the remaining nucleotides contain 2′-O-methyl modifications in ribose moieties.

In certain embodiments, the one or more, optionally one, unmodified nucleotide represents any of the nucleotides of the nucleic acid linker portion as defined previously herein, optionally the nucleotide of the nucleic acid linker portion as defined previously herein that is adjacent to (i) the third nucleic acid portion of (c); and or (ii) the fourth nucleic acid portion of (d); and/or (iii), to the extent present, the passenger nucleic acid portions as defined previously herein.

In certain embodiments, at least one vinylphosphonate modification, such as at least one vinylphosphonate modification in the 5′ region of (i) the first nucleic acid portion of (a); and/or (ii) the second nucleic acid portion of (b); and/or (iii), to the extent present, the 1 to 8 additional nucleic acid portions as defined previously herein.

In certain embodiments, one or more nucleotides of the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions as defined previously herein; and/or
to the extent present, the passenger nucleic acid portions as defined previously herein;
is an inverted nucleotide and is attached to the adjacent nucleotide via the 3′ carbon of the nucleotide and the 3′ carbon of the adjacent nucleotide, and/or is an inverted nucleotide and is attached to the adjacent nucleotide via the 5′ carbon of the nucleotide and the 5′ carbon of the adjacent nucleotide.

In certain embodiments, the inverted nucleotide is attached to the adjacent nucleotide via a phosphate group by way of a phosphodiester linkage; or is attached to the adjacent nucleotide via a phosphorothioate group; or is attached to the adjacent nucleotide via a phosphorodithioate group.

In certain embodiment, the nucleic acid construct is blunt ended.

### In Certain Embodiments,

the first nucleic acid portion of (a); and/or
the second nucleic acid portion of (b); and/or
the third nucleic acid portion of (c); and/or
the fourth nucleic acid portion of (d); and/or
to the extent present, the 1 to 8 additional nucleic acid portions as defined previously herein; and/or
to the extent present, the passenger nucleic acid portions as defined previously herein; has an overhang.

In certain embodiments, the target RNA is an mRNA or an other RNA molecule.

### Compositions and Pharmaceutical Compositions

According to a second aspect, the present disclosure is directed to a composition comprising a construct according to the first aspect, and a physiologically acceptable excipient.

According to a third aspect, the present disclosure is directed to a pharmaceutical composition comprising a construct according to the first aspect.

In certain embodiments, the pharmaceutical composition further comprises a pharmaceutically acceptable excipient, diluent, antioxidant, and/or preservative.

In certain embodiments, the construct is the only pharmaceutically active agent.

In certain embodiments, the pharmaceutical composition is to be administered to patients or individuals which are statin-intolerant and/or for whom statins are contraindicated.

In certain embodiments, the pharmaceutical composition furthermore comprises one or more further pharmaceutically active agents.

In certain embodiments, the further pharmaceutically active agent(s) is/are an RNAi agent which is directed to a target different from TMPRSS6 and from APOC3.

In certain embodiments, the construct and the further pharmaceutically active agent(s) are to be administered concomitantly or in any order.

Diseases to be Treated by muRNA Nucleic Acid Constructs of the Present Disclosure

According to a fourth aspect, the present disclosure is directed to a construct according to the first aspect, for use in human or veterinary medicine or therapy.

According to a fifth aspect, the present disclosure is directed to a construct according to the first aspect, for use in a method of treating a disease or disorder.

According to a sixth aspect, the present disclosure is directed to a method of treating a disease or disorder comprising administration of a construct according to the first aspect, to an individual in need of treatment.

According to a seventh aspect, the present disclosure is directed to a use of a nucleic acid construct according to the first aspect in the manufacture of a medicament for a treatment of a disease or disorder.

In certain embodiments, the disease or disorder is a TMPRSS6- and/or an APOC3-associated disease or disorder or a disease or disorder requiring reduction of TMPRSS6 and/or APOC3 expression levels.

### In Certain Embodiments, the Disease or Disorder is a

(a) a TMPRSS6-associated disease or disorder; a disease or disorder associated with excess accumulation of iron and/or requiring reduction of iron levels such as transfusional iron overlaod, excess parenteral iron supplement, and excess dietary iron intake; a disease or disorder selected from blood disorders such as hemochromatosis, anaemia, thalassaemia, porphyria, and hemosiderosis; bone marrow failure syndromes and myelodysplasia; neurological disorders such as Parkinson's disease, Alzheimer's disease, and Friedreich's ataxia; and/or chronic liver diseases; and/or
(b) an APOC3-associated disease or disorder, or a disease or disorder requiring reduction of APOC3 expression levels, the disease or disorder optionally being selected from dyslipidemia including mixed dyslipidemia; hyperchylomicronemia including familial hyperchylomicronemia; hypertriglyceridemia, optionally severe hypertriglyceridemia and/or hypertriglyceridemia with blood triglyceride levels above 500 mg/dl; inflammation including low-grade inflammation; atherosclerosis; atherosclerotic cardiovascular diseases (ASCVD) including major adverse cardiovascular events (MACE) such as myocardial infarction, stroke and peripheral arterial disease; and pancreatitis including acute pancreatitis.

TMPRSS6 associated hemochromatosis includes, but is not limited to, hereditary hemochromatosis, idiopathic hemochromatosis, primary hemochromatosis, secondary hemochromatosis, severe juvenile hemochromatosis, and neonatal hemochromatosis.

TMPRSS6 associated anemia includes, but is not limited to sideroblastic anemia, hemolytic anemia, dyserythropoietic anemia, congenital dyserythropoietic anemia, hereditary anemia, myelodysplastic syndrome, severe chronic hemolysis, hereditary hemorrhagic telangiectasia, Fanconi anemia, Diamond Blackfan anemia, Shwachman Diamond syndrome, red cell membrane disorders, glucose-6-phosphate dehydrogenase deficiency, and sickle-cell anemia.

TMPRSS6 associated thalassaemia includes hereditary thalassemia, β-thalassemia such as β-thalassemia major and β-thalassemia intermedia, α-thalassemia, □-thalassemia, non-transfusion dependent thalassemia (NTDT), and sickle cell disease.

TMPRSS6 associated porphyria includes porphyria cutanea tarda, and erythropoietic porphyria.

TMPRSS6 associated hemosiderosis includes idiopathic pulmonary hemosiderosis, and renal hemosiderosis.

Further TMPRSS6 associated diseases and disorders include hemoglobinopathy, atransferrinemia, hereditary tyrosinemia, cerebrohepatorenal syndrome, diabetes, glucose intolerance, cardiovascular diseases, hepatic injury, and steatohepatitis.

In certain embodiment, the method comprises administration of a construct according the first aspect, to an individual in need of treatment.

In certain embodiments, the construct is administered subcutaneously or intravenously to the individual, optionally subcutaneously.

In certain embodiments, subsequent to in vivo administration the construct disassembles to yield at least first and second discrete nucleic acid targeting molecules that target portions of RNA transcribed from a TMPRSS6 and an APOC3 gene, respectively.

In particular, due to the nature of the muRNA constructs including a first portion of linked nucleotides, e.g. an antisense sequence, which targets a TMPRSS6 gene and a second portion of linked nucleotides, e.g. an antisense sequence, which targets an APOC3 gene, it is plausible that the following diseases or disorders associated to TMPRSS6 and associated to APOC3 can be treated at the same time with the same molecule, i.e. the nucleic acid constructs disclosed herein.

### TMPRSS6-Associated Disease or Disorder

In particular, the disease or disorder is a TMPRSS6-associated disease or disorder requiring reduction of TMPRSS6 expression levels. Especially, disease or disorder is associated with iron overload and/or a disorder of ineffective erythropoiesis.

The disease or disorder may be a TMPRSS6-associated disease or disorder, wherein the disease or disorder is selected from the group consisting of a TMPRSS6-associated disease or disorder; a disease or disorder associated with excess accumulation of iron and/or requiring reduction of iron levels such as transfusional iron overload, excess parenteral iron supplement, and excess dietary iron intake; a disease or disorder selected from blood disorders such as hemochromatosis, anaemia, thalassaemia, porphyria, and hemosiderosis; bone marrow failure syndromes and myelodysplasia; neurological disorders such as Parkinson's disease, Alzheimer's disease, and Friedreich's ataxia; and/or chronic liver diseases.

In certain embodiments, the nucleic acid construct is administered at a dose of about 0.05 mg/kg to about 50.0 mg/kg, optionally 0.05 mg/kg to about 30.0 mg/kg or 10 mg/kg to about 50 mg/kg of body weight of the human subject.

In certain embodiments, the administering results in a reduction of lipid levels, including triglyceride levels, cholesterol levels, insulin resistance, glucose levels or a combination thereof.

The fact that the Examples compounds show TMPRSS6 knockdown renders it possible such compounds may be used in treating such diseases. This is because reducing TMPRSS6 levels is also at least credibly and plausibly connected with a reduction of triglyceride levels and/or cholesterol levels.

### APOC3-Associated Disease or Disorder

In certain embodiments, an APOC3-associated disease or disorder, or a disease or disorder requiring reduction of APOC3 expression levels, may be selected from dyslipidemia including mixed dyslipidemia; hyperchylomicronemia including familial hyperchylomicronemia; hypertriglyceridemia, optionally severe hypertriglyceridemia and/or hypertriglyceridemia with blood triglyceride levels above 500 mg/dl; inflammation including low-grade inflammation; atherosclerosis; atherosclerotic cardiovascular diseases (ASCVD) including major adverse cardiovascular events (MACE) such as myocardial infarction, stroke and peripheral arterial disease; and pancreatitis including acute pancreatitis.

In certain embodiments, the nucleic acid construct is administered at a dose of about 0.05 mg/kg to about 50.0 mg/kg, optionally 0.05 mg/kg to about 30.0 mg/kg or 10 mg/kg to about 50 mg/kg of body weight of the human subject.

### Process for Making the Constructs

According to a nineth aspect, the present disclosure is directed to a process of making a construct according to the first aspect.

In certain embodiments, the process comprises the steps of:
(i) synthesizing each of:
(a) a first nucleic acid portion that is at least partially complementary to at least a first portion of RNA transcribed from a target gene, such as TMPRSS6;
(b) a second nucleic acid portion that is at least partially complementary to at least a second portion of RNA transcribed from a target gene, which target gene may be the same or different to the target gene defined in (a), wherein optionally the target gene being APOC3;
(c) a third nucleic acid portion that is at least partially complementary to the first nucleic acid portion of (a);
(d) a fourth nucleic acid portion that is at least partially complementary to the second nucleic acid portion of (b);
(ii) contacting at least the first and second nucleic acid portions of (a) and (b) in vitro, so as to form a first nucleic acid duplex region comprising the first and second nucleic acid portions of (a) and (b);
(iii) contacting at least the third and fourth nucleic acid portions of (c) and (d) in vitro, so as to form a second nucleic acid duplex region comprising the third and fourth nucleic acid portions of (c) and (d);
(iv) forming a nucleic acid construct in vitro comprising at least the first and second nucleic acid duplex regions.

In certain embodiments, the process further comprises generating from the construct at least first and second nucleic acid targeting molecules, wherein the first nucleic acid targeting molecule is capable of modulating expression of the target gene of (a), and comprises, or is derived from, at least the first nucleic acid portion of (a), and wherein the second nucleic acid targeting molecule is capable of modulating expression of the target gene of (b), and comprises, or is derived from, the second nucleic acid portion of (b).

In certain embodiments, the at least first and second nucleic acid targeting molecules are generated subsequent to in vivo administration.

In certain embodiments, the labile functionality present in the construct is cleaved subsequent to in vivo administration so as to generate the at least first and second discrete nucleic acid targeting molecules.

In certain embodiments, the labile functionality comprises one or more unmodified nucleotides.

In certain embodiments, the one or more unmodified nucleotides of the labile functionality represent one or more cleavage positions within the construct whereby subsequent to in vivo administration the construct is cleaved at the one or more cleavage positions so as to yield the at least first and second discrete nucleic acid targeting molecules.

In certain embodiments, the cleavage positions are respectively located within the construct so that subsequent to cleavage the first discrete nucleic acid targeting molecule comprises, or is derived from, the first nucleic acid duplex region, and the second discrete nucleic acid targeting molecule comprises, or is derived from, the second nucleic acid duplex region.

### Sequences of the Disclosed Nucleic Acid Constructs

The following Tables show nucleobase sequences and full definitions (including sugar modifications and, where applicable, phosphate modifications) of portions as well as of entire constructs in accordance with the disclosure.

The notation used is common in the art and as the following meaning:
A represents adenine;
U represents uracil;
C represents cytosine;
G represents guanine.
P represents a terminal phosphate group which is optional but not indispensable; m represents a methyl modification at the 2′ position of the sugar of the underlying nucleoside, wherein an accordingly modified nucleotide such as mG is sometimes displayed in brackets ([mG]);
f represents a fluoro modification at the 2′ position of the sugar of the underlying nucleoside, wherein an accordingly modified nucleotide such as fG is sometimes displayed in brackets ([fG]);
r indicates an unmodified (2′-OH) ribonucleotide, wherein corresponding nucleotide such as rG is sometimes displayed in brackets ([rG]);
(ps), #, [#], or * represents a phosphorothioate inter-nucleoside linkage;
i represents an inverted inter-nucleoside linkage, which can be either 3′-3′, or 5′-5′;
vp represents vinyl phosphonate;
mvp represents methyl vinyl phosphonate;
3xGalNAc represents a trivalent GalNAc which is optional but not indispensable; and Mono-GalNAc-PA, which is optional but not indispensable, represents one of optionally three GalNAc bearing moieties, the assembly of three Mono-GalNAc-PA moieties also being referred to as “toothbrush”, wherein the individual moieties are connected by phosphoramidates (“PA”); see the embodiments for an illustration.

Table 1 below shows the nucleobase sequences of APOC3-targeting antisense portions (second nucleic acid portions). The sequences are those of SEQ ID NOs: 8 to 14 (same order). The nucleobase sequence of a further APOC3-targeting antisense portion of the disclosure is set forth in SEQ ID NO: 29 (below Table 1).

| SEQ ID | Experimental | Anti-Sense Sequence |
|---|---|---|
| No. | denotation | (5′ to 3′) |
| 8 | AP277 | UUGGAUAGGCAGGUGGACU |
| 9 | AP337 | UGCACUGAGAAUACUGUCC |
| 10 | AP028 | UCAACAAGGAGUACCCGGG |
| 11 | AP369 | UCUUGUCCAGCUUUAUUGG |
| 12 | AP366 | UUCCAGCUUUAUUGGGAGG |
| 13 | AP367 | UGUCCAGCUUUAUUGGGAG |
| 14 | AP336 | UCACUGAGAAUACUGUCCC |
| 29 | AP-as | UGCACUGAGAAUACUGUCC |

Table 2 below shows the nucleobase sequences of APOC3-targeting sense portions (fourth nucleic acid portions of the disclosure). The sequences are those of SEQ ID NOs: 22 to 28 (same order). The nucleobase sequence of a further APOC3-targeting sense portion of the disclosure is set forth in SEQ ID NO: 30 (below Table 2).

| SEQ ID | Experimental | SS Sequence |
|---|---|---|
| No. | denotation | (5′ to 3′) |
| 22 | AP277 | CACCUGCCUAUCCAA |
| 23 | AP337 | AGUAUUCUCAGUGCA |
| 24 | AP028 | GGUACUCCUUGUUGA |
| 25 | AP369 | UAAAGCUGGACAAGA |
| 26 | AP366 | CCAAUAAAGCUGGAA |
| 27 | AP367 | CAAUAAAGCUGGACA |
| 28 | AP336 | CAGUAUUCUCAGUGA |
| 30 | AP-s | GUAUUCUCAGUGCA |

### In Certain Embodiments, the Following Sequences May be Used:

SEQ ID No. 31:
UGUACCCUAGGAAAUACCAGUACUCCUUGUUGA
SEQ ID No. 32:
UCAACAAGGAGUACCCGGGAUUUCCUAGGGUACA
SEQ ID No. 33:
UGUACCCUAGGAAAUACCAGGUACUCCUUGUUGA

Table 3a shows TMPRSS6-targeting antisense portions including modification information.

| SEQ ID | |
|---|---|
| No. | AS modified |
| 654 | [mU][#][fG][#][mU][fA][mC][fC][fU][mA][fG] |
| | [mG][fA][mA][fA][mU][#][fA][#][mC][#][fC] |
| | [#][rA] |

Table 3b shows APOC3-targeting antisense portions including modification information.

| SEQ ID | |
|---|---|
| No. | AS Modified |
| 655 | PmU.fU.mG.fG.mA.fU.mA.fG.mG.fC.mA.fG.mG.fU.mG.fG.mA.fC.mU |
| 656 | PmU.fG.mC.fA.mC.fU.mG.fA.mG.fA.mA.fU.mA.fC.mU.fG.mU.fC.mC |
| 657 | PmU.fC.mA.fA.mC.fA.mA.fG.mG.fA.mG.fU.mA.fC.mC.fC.mG.fG.mG |
| 658 | PmU.fC.mU.fU.mG.fU.mC.fC.mA.fG.mC.fU.mU.fU.mA.fU.mU.fG.mG |
| 659 | PmU.fU.mC.fC.mA.fG.mC.fU.mU.fU.mA.fU.mU.fG.mG.fG.mA.fG.mG |
| 660 | PmU.fG.mU.fC.mC.fA.mG.fC.mU.fU.mU.fA.mU.fU.mG.fG.mG.fA.mG |
| 661 | PmU.fC.mA.fC.mU.fG.mA.fG.mA.fA.mU.fA.mC.fU.mG.fU.mC.fC.mC |

Table 4a shows TMPRSS6-targeting sense portions including modification information.

| SEQ ID | |
|---|---|
| No. | S Modified |
| 662 | [fA][#][mU][#][fU][mU][fC][mC][fU][mA][fG] |
| | [mG][fG][mU][fA][#][mC][#][fA] |

Table 4b shows APOC3-targeting sense portions including modification information.

| SEQ | |
|---|---|
| ID | |
| No. | S Modified |
| 663 | fC.mA.fC.mC.fU.mG.fC.mC.fU.mA.fU.mC.fC.mA.fA |
| 664 | fA.mG.fU.mA.fU.mU.fC.mU.fC.mA.fG.mU.fG.mC.fA |
| 665 | fG.mG.fU.mA.fC.mU.fC.mC.fU.mU.fG.mU.fU.mG.fA |
| 666 | fU.mA.fA.mA.fG.mC.fU.mG.fG.mA.fC.mA.fA.mG.fA |
| 667 | fC.mC.fA.mA.fU.mA.fA.mA.fG.mC.fU.mG.fG.mA.fA |
| 668 | fC.mA.fA.mU.fA.mA.fA.mG.fC.mU.fG.mG.fA.mC.fA |
| 669 | fC.mA.fG.mU.fA.mU.fU.mC.fU.mC.fA.mG.fU.mG.fA |

Table 5a shows linked first and fourth nucleic acid portions of the disclosure. Linking is direct to give rise to a single contiguous strand.

| SEQ ID | |
|---|---|
| No. | First strand, modified |
| 670 | [mU][#][fG][#][mU][fA][mC][fC][mC][fU][mA][fG][mG][fA][mA][fA][mU][#][fA] |
| | [#][mC][#][fC][#][rA][mG][#][fU][#][mA][fC][mU][fC][mC][fU][mU][fG] |
| 671 | [mU][#][fG][#][mU][fA][mC][fC][mC][fU][mA][fG][mG][fA][mA][fA][#][mU][#] |
| | [fA][#][mC][#][fC][#][rA][fG][#][mG][#][fU][mA][fC][mU][fC][mC][fU][mU] |
| | [fG][mU][fU][#][mG][#][fA][#][3XGaINAc] |

Table 5b shows linked second and third nucleic acid portions of the disclosure. Linking is direct to give rise to a single contiguous strand.

| SEQ ID | |
|---|---|
| No. | Second strand, modified |
| 672 | [mU][#][fC][#][mA][fA][mC][fA][mA][fG][mG][fA][mG][fU][mA][fC][#][mC][#][fC][#] |
| | [mG][#][fG][#][rG][fA][#][mU][#][fU][mU][fC][mC][fU][mA][fG][mG][fG][mU][fA][#] |
| | [mC][#][fA][#][3XGaINAc] |

Table 6a below shows the nucleobase sequences of TMPRSS6-targeting antisense sequences (first nucleic acid portions of muRNA or antisense sequence of mxRNA).

| | | Target | Antisense sequence |
|---|---|---|---|
| | | position | (19 mer; from 5′ |
| SEQ | | in | terminus (left) to |
| ID | Experimental | TMPRSS6 | 3′ terminus |
| NO: | denotation | mRNA | (right)) |
| 34 | TMPRSS6_1(mx) | 1930 | UCCCAGCGGUCAGCGAUGA |
| 35 | TMPRSS6_2(mx) | 1971 | UGGCCAUGCUGUCCUCCUG |
| 36 | TMPRSS6_3(mx) | 2053 | UGGCUCACCUUGAAGGACA |
| 37 | TMPRSS6_4(mx) | 1970 | UGCCAUGCUGUCCUCCUGG |
| 38 | TMPRSS6_5(mx) | 1810 | UCCUGGAGGCCACAGUCAC |
| 39 | TMPRSS6_6(mx) | 1931 | UACCCAGCGGUCAGCGAUG |
| 40 | TMPRSS6_7(mx) | 1972 | UAGGCCAUGCUGUCCUCCU |
| 41 | TMPRSS6_8(mx) | 2049 | UCACCUUGAAGGACACCUC |
| 42 | TMPRSS6_9(mx) | 330 | UGUACCCUAGGAAAUACCA |
| 43 | TMPRSS6_10(mx) | 1418 | UGUGAGGGAGAUCUGGGAG |
| 44 | TMPRSS6_11(mx) | 1413 | UGGAGAUCUGGGAGGUGAA |
| 45 | TMPRSS6_12(mx) | 1929 | UCCAGCGGUCAGCGAUGAG |
| 46 | TMPRSS6_13(mx) | 1322 | UAGCCUCCUGUUCUGGAUC |
| 47 | TMPRSS6_14(mx) | 1967 | UAUGCUGUCCUCCUGGAAG |
| 48 | TMPRSS6_15(mx) | 1415 | UAGGGAGAUCUGGGAGGUG |
| 49 | TMPRSS6_16(mx) | 1968 | UCAUGCUGUCCUCCUGGAA |
| 50 | TMPRSS6_17(mx) | 2052 | UGCUCACCUUGAAGGACAC |
| 51 | TMPRSS6_18(mx) | 1966 | UUGCUGUCCUCCUGGAAGC |
| 52 | TMPRSS6_19(mx) | 1969 | UCCAUGCUGUCCUCCUGGA |
| 53 | TMPRSS6_20(mx) | 1416 | UGAGGGAGAUCUGGGAGGU |
| 54 | TMPRSS6_21(mx) | 331 | UUGUACCCUAGGAAAUACC |
| 55 | TMPRSS6_22(mx) | 332 | UUUGUACCCUAGGAAAUAC |
| 56 | TMPRSS6_23(mx) | 1808 | UUGGAGGCCACAGUCACAG |
| 57 | TMPRSS6_24(mx) | 1974 | UGGAGGCCAUGCUGUCCUC |
| 58 | TMPRSS6_25(mx) | 2050 | UUCACCUUGAAGGACACCU |
| 59 | TMPRSS6_26(mx) | 2051 | UCUCACCUUGAAGGACACC |
| 60 | TMPRSS6_27(mx) | 1807 | UGGAGGCCACAGUCACAGU |
| 61 | TMPRSS6_28(mx) | 1965 | UGCUGUCCUCCUGGAAGCA |
| 62 | TMPRSS6_29(mx) | 556 | UACCAGAAGAAGCAGGUGA |
| 63 | TMPRSS6_30(mx) | 1325 | UCACAGCCUCCUGUUCUGG |
| 64 | TMPRSS6_31(mx) | 1327 | UCACACAGCCUCCUGUUCU |
| 65 | TMPRSS6_32(mx) | 1562 | UCAGUUUCUCUCAUCCAGG |
| 66 | TMPRSS6_33(mx) | 1234 | UCCAAGCCGUAGUCCAGAG |
| 67 | TMPRSS6_34(mx) | 557 | UAACCAGAAGAAGCAGGUG |
| 68 | TMPRSS6_35(mx) | 555 | UCCAGAAGAAGCAGGUGAG |
| 69 | TMPRSS6_36(mx) | 460 | UGCAUCUUCUGGGCUUUGG |
| 70 | TMPRSS6_37(mx) | 1233 | UCAAGCCGUAGUCCAGAGA |
| 71 | TMPRSS6_38(mx) | 1861 | UGCCACUCACCCUCGGAGG |
| 72 | TMPRSS6_39(mx) | 1326 | UACACAGCCUCCUGUUCUG |
| 73 | TMPRSS6_40(mx) | 1235 | UGCCAAGCCGUAGUCCAGA |
| 74 | TMPRSS6_41(mx) | 2426 | UAGGAACCAGCGGCCACUG |
| 75 | TMPRSS6_42(mx) | 1857 | UCUCACCCUCGGAGGACAC |
| 76 | TMPRSS6_43(mx) | 1858 | UACUCACCCUCGGAGGACA |
| 77 | TMPRSS6_44(mx) | 461 | UAGCAUCUUCUGGGCUUUG |
| 78 | TMPRSS6_45(mx) | 1859 | UCACUCACCCUCGGAGGAC |
| 79 | TMPRSS6_46(mx) | 2027 | UGGCCAGCGCGAGUUCUGC |
| 80 | TMPRSS6_47(mx) | 462 | UGAGCAUCUUCUGGGCUUU |
| 81 | TMPRSS6_48(mx) | 1862 | UGGCCACUCACCCUCGGAG |
| 82 | TMPRSS6_49(mx) | 1324 | UACAGCCUCCUGUUCUGGA |
| 83 | TMPRSS6_50(mx) | 1561 | UAGUUUCUCUCAUCCAGGC |
| 84 | TMPRSS6_51(mx) | 1560 | UGUUUCUCUCAUCCAGGCC |
| 85 | TMPRSS6_52(mx) | 1323 | UCAGCCUCCUGUUCUGGAU |
| 86 | TMPRSS6_53(mx) | 1866 | UCCAUGGCCACUCACCCUC |
| 87 | TMPRSS6_54(mx) | 1867 | UGCCAUGGCCACUCACCCU |
| 88 | TMPRSS6_55(mx) | 2358 | UCUUGCCCUUGCGGUAGCC |
| 89 | TMPRSS6_56(mx) | 2360 | UUUCUUGCCCUUGCGGUAG |
| 90 | TMPRSS6_57(mx) | 1231 | UAGCCGUAGUCCAGAGAGG |
| 91 | TMPRSS6_58(mx) | 1865 | UCAUGGCCACUCACCCUCG |
| 92 | TMPRSS6_59(mx) | 1328 | UCCACACAGCCUCCUGUUC |
| 93 | TMPRSS6_60(mx) | 1863 | UUGGCCACUCACCCUCGGA |
| 94 | TMPRSS6_61(mx) | 1230 | UGCCGUAGUCCAGAGAGGG |
| 95 | TMPRSS6_62(mx) | 1860 | UCCACUCACCCUCGGAGGA |
| 96 | TMPRSS6_63(mx) | 1868 | UUGCCAUGGCCACUCACCC |
| 97 | TMPRSS6_64(mx) | 2359 | UUCUUGCCCUUGCGGUAGC |
| 98 | TMPRSS6_65(mx) | 1484 | UAGGAACUCUCCAGGGCAG |
| 99 | TMPRSS6_66(mx) | 329 | UUACCCUAGGAAAUACCAG |
| 100 | TMPRSS6_67(mx) | 1805 | UAGGCCACAGUCACAGUGC |
| 101 | TMPRSS6_68(mx) | 338 | UUCCGCCUUGUACCCUAGG |
| 102 | TMPRSS6_69(mx) | 2057 | UAGGCGGCUCACCUUGAAG |
| 103 | TMPRSS6_70(mx) | 1485 | UGAGGAACUCUCCAGGGCA |
| 104 | TMPRSS6_71(mx) | 1555 | UUCUCAUCCAGGCCGUUGG |
| 105 | TMPRSS6_72(mx) | 337 | UCCGCCUUGUACCCUAGGA |
| 106 | TMPRSS6_73(mx) | 777 | UCACGUAGCUGUAGCGGUA |
| 107 | TMPRSS6_74(mx) | 2056 | UGGCGGCUCACCUUGAAGG |
| 108 | TMPRSS6_75(mx) | 560 | UAUGAACCAGAAGAAGCAG |
| 109 | TMPRSS6_76(mx) | 327 | UCCCUAGGAAAUACCAGAG |
| 110 | TMPRSS6_77(mx) | 775 | UCGUAGCUGUAGCGGUAAC |
| 111 | TMPRSS6_78(mx) | 335 | UGCCUUGUACCCUAGGAAA |
| 112 | TMPRSS6_79(mx) | 1804 | UGGCCACAGUCACAGUGCU |
| 113 | TMPRSS6_80(mx) | 846 | UCUGCAGGUGCCACAGGCA |
| 114 | TMPRSS6_81(mx) | 776 | UACGUAGCUGUAGCGGUAA |
| 115 | TMPRSS6_82(mx) | 1556 | UCUCUCAUCCAGGCCGUUG |
| 116 | TMPRSS6_83(mx) | 328 | UACCCUAGGAAAUACCAGA |
| 117 | TMPRSS6_84(mx) | 774 | UGUAGCUGUAGCGGUAACA |
| 118 | TMPRSS6_85(mx) | 2055 | UGCGGCUCACCUUGAAGGA |
| 119 | TMPRSS6_86(mx) | 334 | UCCUUGUACCCUAGGAAAU |
| 120 | TMPRSS6_87(mx) | 333 | UCUUGUACCCUAGGAAAUA |
| 121 | TMPRSS6_88(mx) | 843 | UCAGGUGCCACAGGCAGCU |
| 122 | TMPRSS6_89(mx) | 2971 | UCAGAUCCCAAGUUAGACC |
| 123 | TMPRSS6_90(mx) | 1567 | UAAACGCAGUUUCUCUCAU |
| 124 | TMPRSS6_91(mx) | 2024 | UCAGCGCGAGUUCUGCCAC |
| 125 | TMPRSS6_92(mx) | 3165 | UAGCUUUAUUCCAAAGGGC |
| 126 | TMPRSS6_93(mx) | 321 | UGAAAUACCAGAGUAGCAC |
| 127 | TMPRSS6_94(mx) | 3154 | UAAAGGGCAGCUGAGCUCA |
| 128 | TMPRSS6_95(mx) | 928 | UCCACGUCAUACAUGGCCA |
| 129 | TMPRSS6_96(mx) | 526 | UCAAAGGAAUAGACGGAGC |
| 130 | TMPRSS6_97(mx) | 1927 | UAGCGGUCAGCGAUGAGGG |
| 131 | TMPRSS6_98(mx) | 737 | UGCAGCUAUGUCUUUCACA |
| 132 | TMPRSS6_99(mx) | 3166 | UCAGCUUUAUUCCAAAGGG |
| 133 | TMPRSS6_100(mx) | 1243 | UACCAGAGGGCCAAGCCGU |
| 134 | TMPRSS6_101(mx) | 1280 | UAAAUCAUACUUCUGCCUC |
| 135 | TMPRSS6_102(mx) | 2541 | UGGCAGUUCCUCAGGUCAC |
| 136 | TMPRSS6_103(mx) | 446 | UUUGGCGGUUUCACUGCGG |
| 137 | TMPRSS6_104(mx) | 738 | UUGCAGCUAUGUCUUUCAC |
| 138 | TMPRSS6_105(mx) | 451 | UGGGCUUUGGCGGUUUCAC |
| 139 | TMPRSS6_106(mx) | 1707 | UCUGGAAGGUGAAUGUCCC |
| 140 | TMPRSS6_107(mx) | 2849 | UCAUUCUUGCUGCUGAGCC |
| 141 | TMPRSS6_108(mx) | 638 | UGACAGCAGCUCCUCCACC |
| 142 | TMPRSS6_109(mx) | 1044 | UCAGGCCCUUCUUCCAGAC |
| 143 | TMPRSS6_110(mx) | 2851 | UAGCAUUCUUGCUGCUGAG |
| 144 | TMPRSS6_111(mx) | 520 | UAAUAGACGGAGCUGGAGU |
| 145 | TMPRSS6_112(mx) | 1060 | UGGUCGUAGUAGCUGUGCA |
| 146 | TMPRSS6_113(mx) | 2903 | UAGCCUCUGUACAGAGUGG |
| 147 | TMPRSS6_114(mx) | 734 | UGCUAUGUCUUUCACACUG |
| 148 | TMPRSS6_115(mx) | 413 | UCGGCGGGUAAGAUCCUGG |
| 149 | TMPRSS6_116(mx) | 1945 | UGGGCAGCUGUUAUCACCC |
| 150 | TMPRSS6_117(mx) | 1568 | UCAAACGCAGUUUCUCUCA |
| 151 | TMPRSS6_118(mx) | 2494 | UUGAUGCGGGUGUAGACGC |
| 152 | TMPRSS6_119(mx) | 1099 | UAGGCCUGGAAGACCACCG |
| 153 | TMPRSS6_120(mx) | 736 | UCAGCUAUGUCUUUCACAC |
| 154 | TMPRSS6_121(mx) | 3167 | UGCAGCUUUAUUCCAAAGG |
| 155 | TMPRSS6_122(mx) | 1281 | UCAAAUCAUACUUCUGCCU |
| 156 | TMPRSS6_123(mx) | 2039 | UGACACCUCUCCAGGCCAG |
| 157 | TMPRSS6_124(mx) | 523 | UAGGAAUAGACGGAGCUGG |
| 158 | TMPRSS6_125(mx) | 1582 | UGGAAUGUGGCUCUGCAAA |
| 159 | TMPRSS6_126(mx) | 2527 | UUCACCACUUGCUGGAUCC |
| 160 | TMPRSS6_127(mx) | 1446 | UGCCAUAGUGCACCCGCAC |
| 161 | TMPRSS6_128(mx) | 830 | UCAGCUGGAGGCCAGGUGG |
| 162 | TMPRSS6_129(mx) | 2697 | UCAUCACUGGAGCAGACAU |
| 163 | TMPRSS6_130(mx) | 1944 | UGGCAGCUGUUAUCACCCA |
| 164 | TMPRSS6_131(mx) | 1310 | UUGGAUCGUCCACUGGCCC |
| 165 | TMPRSS6_132(mx) | 234 | UUUUUCUCUUGGAGUCCUC |
| 166 | TMPRSS6_133(mx) | 890 | UAGCGUCCACUCCAGCCGG |
| 167 | TMPRSS6_134(mx) | 320 | UAAAUACCAGAGUAGCACC |
| 168 | TMPRSS6_135(mx) | 2353 | UCCUUGCGGUAGCCGGCAC |
| 169 | TMPRSS6_136(mx) | 2492 | UAUGCGGGUGUAGACGCCG |
| 170 | TMPRSS6_137(mx) | 448 | UCUUUGGCGGUUUCACUGC |
| 171 | TMPRSS6_138(mx) | 729 | UGUCUUUCACACUGGCUUC |
| 172 | TMPRSS6_139(mx) | 3155 | UCAAAGGGCAGCUGAGCUC |
| 173 | TMPRSS6_140(mx) | 2532 | UUCAGGUCACCACUUGCUG |
| 174 | TMPRSS6_141(mx) | 1564 | UCGCAGUUUCUCUCAUCCA |
| 175 | TMPRSS6_142(mx) | 654 | UCGAGCUGUUGACUGUGGA |
| 176 | TMPRSS6_143(mx) | 2475 | UGAAGUAGUUAGGCCGGCC |
| 177 | TMPRSS6_144(mx) | 2041 | UAGGACACCUCUCCAGGCC |
| 178 | TMPRSS6_145(mx) | 733 | UCUAUGUCUUUCACACUGG |
| 179 | TMPRSS6_146(mx) | 2501 | UACACCUGUGAUGCGGGUG |
| 180 | TMPRSS6_147(mx) | 1566 | UAACGCAGUUUCUCUCAUC |
| 181 | TMPRSS6_148(mx) | 2295 | UGCACAGGUCCUGUGGGAU |
| 182 | TMPRSS6_149(mx) | 2531 | UCAGGUCACCACUUGCUGG |
| 183 | TMPRSS6_150(mx) | 235 | UCUUUUCUCUUGGAGUCCU |
| 184 | TMPRSS6_151(mx) | 956 | UGUGAUGAGCCUCUUCUCC |
| 185 | TMPRSS6_152(mx) | 2040 | UGGACACCUCUCCAGGCCA |
| 186 | TMPRSS6_153(mx) | 571 | UGGAUUUGGAGAAUGAACC |
| 187 | TMPRSS6_154(mx) | 1444 | UCAUAGUGCACCCGCACAC |
| 188 | TMPRSS6_155(mx) | 2979 | UUCCAUUCCCAGAUCCCAA |
| 189 | TMPRSS6_156(mx) | 735 | UAGCUAUGUCUUUCACACU |
| 190 | TMPRSS6_157(mx) | 2660 | UCACCUCCUGCCACCACAG |
| 191 | TMPRSS6_158(mx) | 1319 | UCUCCUGUUCUGGAUCGUC |
| 192 | TMPRSS6_159(mx) | 2970 | UAGAUCCCAAGUUAGACCA |
| 193 | TMPRSS6_160(mx) | 1738 | UUGGGCUUCUUCACGCAGC |
| 194 | TMPRSS6_161(mx) | 1282 | UGCAAAUCAUACUUCUGCC |
| 195 | TMPRSS6_162(mx) | 409 | UGGGUAAGAUCCUGGGAGA |
| 196 | TMPRSS6_163(mx) | 1227 | UGUAGUCCAGAGAGGGCAC |
| 197 | TMPRSS6_164(mx) | 691 | UGGUCCACUUCGUACUCGG |
| 198 | TMPRSS6_165(mx) | 2669 | UACAAGAUGCCACCUCCUG |
| 199 | TMPRSS6_166(mx) | 322 | UGGAAAUACCAGAGUAGCA |
| 200 | TMPRSS6_167(mx) | 765 | UGCGGUAACAACCCAGCGU |
| 201 | TMPRSS6_168(mx) | 3151 | UGGGCAGCUGAGCUCACCU |
| 202 | TMPRSS6_169(mx) | 709 | UGGAUCACUAGGCCCUCGG |
| 203 | TMPRSS6_170(mx) | 2334 | UCAGCAUGCGUGGCGUCAC |
| 204 | TMPRSS6_171(mx) | 1565 | UACGCAGUUUCUCUCAUCC |
| 205 | TMPRSS6_172(mx) | 2848 | UAUUCUUGCUGCUGAGCCA |
| 206 | TMPRSS6_173(mx) | 1297 | UGGCCCUGGGUGCACGGCA |
| 207 | TMPRSS6_174(mx) | 2025 | UCCAGCGCGAGUUCUGCCA |
| 208 | TMPRSS6_175(mx) | 682 | UCGUACUCGGCCCUGUAGG |
| 209 | TMPRSS6_176(mx) | 2524 | UCCACUUGCUGGAUCCAGC |
| 210 | TMPRSS6_177(mx) | 2286 | UCUGUGGGAUCAACUGCAC |
| 211 | TMPRSS6_178(mx) | 2288 | UUCCUGUGGGAUCAACUGC |
| 212 | TMPRSS6_179(mx) | 1942 | UCAGCUGUUAUCACCCAGC |
| 213 | TMPRSS6_180(mx) | 2972 | UCCAGAUCCCAAGUUAGAC |
| 214 | TMPRSS6_181(mx) | 2477 | UCCGAAGUAGUUAGGCCGG |
| 215 | TMPRSS6_182(mx) | 2485 | UUGUAGACGCCGAAGUAGU |
| 216 | TMPRSS6_183(mx) | 2495 | UGUGAUGCGGGUGUAGACG |
| 217 | TMPRSS6_184(mx) | 339 | UCUCCGCCUUGUACCCUAG |
| 218 | TMPRSS6_185(mx) | 1311 | UCUGGAUCGUCCACUGGCC |
| 219 | TMPRSS6_186(mx) | 1216 | UAGGGCACCGUGAGGUGCC |
| 220 | TMPRSS6_187(mx) | 2482 | UAGACGCCGAAGUAGUUAG |
| 221 | TMPRSS6_188(mx) | 524 | UAAGGAAUAGACGGAGCUG |
| 222 | TMPRSS6_189(mx) | 2850 | UGCAUUCUUGCUGCUGAGC |
| 223 | TMPRSS6_190(mx) | 2009 | UCACACCUUGCCCAGGAAC |
| 224 | TMPRSS6_191(mx) | 957 | UGGUGAUGAGCCUCUUCUC |
| 225 | TMPRSS6_192(mx) | 2668 | UCAAGAUGCCACCUCCUGC |
| 226 | TMPRSS6_193(mx) | 769 | UUGUAGCGGUAACAACCCA |
| 227 | TMPRSS6_194(mx) | 2321 | UGUCACCUGGUAGCGAUAG |
| 228 | TMPRSS6_195(mx) | 730 | UUGUCUUUCACACUGGCUU |
| 229 | TMPRSS6_196(mx) | 2337 | UACACAGCAUGCGUGGCGU |
| 230 | TMPRSS6_197(mx) | 2588 | UUGCCCUGGGCUCUCUGAG |
| 231 | TMPRSS6_198(mx) | 964 | UACACCGAGGUGAUGAGCC |
| 232 | TMPRSS6_199(mx) | 1303 | UUCCACUGGCCCUGGGUGC |
| 233 | TMPRSS6_200(mx) | 341 | UACCUCCGCCUUGUACCCU |

Note
In particular, the first nucleobase at the 5′ terminus in each of the above constructs may be substituted by U.

Table 6b below shows a selection of specific 20mer sense sequences, which can be the basis for the third nucleic acid portion of muRNA, as well as their targeting regions.

| | | Target | Sense sequence |
|---|---|---|---|
| | | position | (20 mer from 5′ |
| SEQ | | in | terminus (left) |
| ID | Experimental | TMPRSS6 | to 3′ terminus |
| NO: | denotation | mRNA | (right)) |
| 234 | TMPRSS6_1(mx) | 1930 | CUCAUCGCUGACCGCUGGGU |
| 235 | TMPRSS6_2(mx) | 1971 | CCAGGAGGACAGCAUGGCCU |
| 236 | TMPRSS6_3(mx) | 2053 | GUGUCCUUCAAGGUGAGCCG |
| 237 | TMPRSS6_4(mx) | 1970 | UCCAGGAGGACAGCAUGGCC |
| 238 | TMPRSS6_5(mx) | 1810 | UGUGACUGUGGCCUCCAGGG |
| 239 | TMPRSS6_6(mx) | 1931 | UCAUCGCUGACCGCUGGGUG |
| 240 | TMPRSS6_7(mx) | 1972 | CAGGAGGACAGCAUGGCCUC |
| 241 | TMPRSS6_8(mx) | 2049 | AGAGGUGUCCUUCAAGGUGA |
| 242 | TMPRSS6_9(mx) | 330 | CUGGUAUUUCCUAGGGUACA |
| 243 | TMPRSS6_10(mx) | 1418 | CCUCCCAGAUCUCCCUCACC |
| 244 | TMPRSS6_11(mx) | 1413 | CUUCACCUCCCAGAUCUCCC |
| 245 | TMPRSS6_12(mx) | 1929 | CCUCAUCGCUGACCGCUGGG |
| 246 | TMPRSS6_13(mx) | 1322 | CGAUCCAGAACAGGAGGCUG |
| 247 | TMPRSS6_14(mx) | 1967 | GCUUCCAGGAGGACAGCAUG |
| 248 | TMPRSS6_15(mx) | 1415 | UCACCUCCCAGAUCUCCCUC |
| 249 | TMPRSS6_16(mx) | 1968 | CUUCCAGGAGGACAGCAUGG |
| 250 | TMPRSS6_17(mx) | 2052 | GGUGUCCUUCAAGGUGAGCC |
| 251 | TMPRSS6_18(mx) | 1966 | UGCUUCCAGGAGGACAGCAU |
| 252 | TMPRSS6_19(mx) | 1969 | UUCCAGGAGGACAGCAUGGC |
| 253 | TMPRSS6_20(mx) | 1416 | CACCUCCCAGAUCUCCCUCA |
| 254 | TMPRSS6_21(mx) | 331 | UGGUAUUUCCUAGGGUACAA |
| 255 | TMPRSS6_22(mx) | 332 | GGUAUUUCCUAGGGUACAAG |
| 256 | TMPRSS6_23(mx) | 1808 | ACUGUGACUGUGGCCUCCAG |
| 257 | TMPRSS6_24(mx) | 1974 | GGAGGACAGCAUGGCCUCCA |
| 258 | TMPRSS6_25(mx) | 2050 | GAGGUGUCCUUCAAGGUGAG |
| 259 | TMPRSS6_26(mx) | 2051 | AGGUGUCCUUCAAGGUGAGC |
| 260 | TMPRSS6_27(mx) | 1807 | CACUGUGACUGUGGCCUCCA |
| 261 | TMPRSS6_28(mx) | 1965 | CUGCUUCCAGGAGGACAGCA |
| 262 | TMPRSS6_29(mx) | 556 | CUCACCUGCUUCUUCUGGUU |
| 263 | TMPRSS6_30(mx) | 1325 | UCCAGAACAGGAGGCUGUGU |
| 264 | TMPRSS6_31(mx) | 1327 | CAGAACAGGAGGCUGUGUGG |
| 265 | TMPRSS6_32(mx) | 1562 | GCCUGGAUGAGAGAAACUGC |
| 266 | TMPRSS6_33(mx) | 1234 | UCUCUGGACUACGGCUUGGC |
| 267 | TMPRSS6_34(mx) | 557 | UCACCUGCUUCUUCUGGUUC |
| 268 | TMPRSS6_35(mx) | 555 | CCUCACCUGCUUCUUCUGGU |
| 269 | TMPRSS6_36(mx) | 460 | GCCAAAGCCCAGAAGAUGCU |
| 270 | TMPRSS6_37(mx) | 1233 | CUCUCUGGACUACGGCUUGG |
| 271 | TMPRSS6_38(mx) | 1861 | UCCUCCGAGGGUGAGUGGCC |
| 272 | TMPRSS6_39(mx) | 1326 | CCAGAACAGGAGGCUGUGUG |
| 273 | TMPRSS6_40(mx) | 1235 | CUCUGGACUACGGCUUGGCC |
| 274 | TMPRSS6_41(mx) | 2426 | UCAGUGGCCGCUGGUUCCUG |
| 275 | TMPRSS6_42(mx) | 1857 | UGUGUCCUCCGAGGGUGAGU |
| 276 | TMPRSS6_43(mx) | 1858 | GUGUCCUCCGAGGGUGAGUG |
| 277 | TMPRSS6_44(mx) | 461 | CCAAAGCCCAGAAGAUGCUC |
| 278 | TMPRSS6_45(mx) | 1859 | UGUCCUCCGAGGGUGAGUGG |
| 279 | TMPRSS6_46(mx) | 2027 | GGCAGAACUCGCGCUGGCCU |
| 280 | TMPRSS6_47(mx) | 462 | CAAAGCCCAGAAGAUGCUCA |
| 281 | TMPRSS6_48(mx) | 1862 | CCUCCGAGGGUGAGUGGCCA |
| 282 | TMPRSS6_49(mx) | 1324 | AUCCAGAACAGGAGGCUGUG |
| 283 | TMPRSS6_50(mx) | 1561 | GGCCUGGAUGAGAGAAACUG |
| 284 | TMPRSS6_51(mx) | 1560 | CGGCCUGGAUGAGAGAAACU |
| 285 | TMPRSS6_52(mx) | 1323 | GAUCCAGAACAGGAGGCUGU |
| 286 | TMPRSS6_53(mx) | 1866 | CGAGGGUGAGUGGCCAUGGC |
| 287 | TMPRSS6_54(mx) | 1867 | GAGGGUGAGUGGCCAUGGCA |
| 288 | TMPRSS6_55(mx) | 2358 | CGGCUACCGCAAGGGCAAGA |
| 289 | TMPRSS6_56(mx) | 2360 | GCUACCGCAAGGGCAAGAAG |
| 290 | TMPRSS6_57(mx) | 1231 | CCCUCUCUGGACUACGGCUU |
| 291 | TMPRSS6_58(mx) | 1865 | CCGAGGGUGAGUGGCCAUGG |
| 292 | TMPRSS6_59(mx) | 1328 | AGAACAGGAGGCUGUGUGGC |
| 293 | TMPRSS6_60(mx) | 1863 | CUCCGAGGGUGAGUGGCCAU |
| 294 | TMPRSS6_61(mx) | 1230 | GCCCUCUCUGGACUACGGCU |
| 295 | TMPRSS6_62(mx) | 1860 | GUCCUCCGAGGGUGAGUGGC |
| 296 | TMPRSS6_63(mx) | 1868 | AGGGUGAGUGGCCAUGGCAG |
| 297 | TMPRSS6_64(mx) | 2359 | GGCUACCGCAAGGGCAAGAA |
| 298 | TMPRSS6_65(mx) | 1484 | CCUGCCCUGGAGAGUUCCUC |
| 299 | TMPRSS6_66(mx) | 329 | UCUGGUAUUUCCUAGGGUAC |
| 300 | TMPRSS6_67(mx) | 1805 | AGCACUGUGACUGUGGCCUC |
| 301 | TMPRSS6_68(mx) | 338 | UCCUAGGGUACAAGGCGGAG |
| 302 | TMPRSS6_69(mx) | 2057 | CCUUCAAGGUGAGCCGCCUG |
| 303 | TMPRSS6_70(mx) | 1485 | CUGCCCUGGAGAGUUCCUCU |
| 304 | TMPRSS6_71(mx) | 1555 | CCCAACGGCCUGGAUGAGAG |
| 305 | TMPRSS6_72(mx) | 337 | UUCCUAGGGUACAAGGCGGA |
| 306 | TMPRSS6_73(mx) | 777 | UUACCGCUACAGCUACGUGG |
| 307 | TMPRSS6_74(mx) | 2056 | UCCUUCAAGGUGAGCCGCCU |
| 308 | TMPRSS6_75(mx) | 560 | CCUGCUUCUUCUGGUUCAUU |
| 309 | TMPRSS6_76(mx) | 327 | ACUCUGGUAUUUCCUAGGGU |
| 310 | TMPRSS6_77(mx) | 775 | UGUUACCGCUACAGCUACGU |
| 311 | TMPRSS6_78(mx) | 335 | AUUUCCUAGGGUACAAGGCG |
| 312 | TMPRSS6_79(mx) | 1804 | GAGCACUGUGACUGUGGCCU |
| 313 | TMPRSS6_80(mx) | 846 | CUGCCUGUGGCACCUGCAGG |
| 314 | TMPRSS6_81(mx) | 776 | GUUACCGCUACAGCUACGUG |
| 315 | TMPRSS6_82(mx) | 1556 | CCAACGGCCUGGAUGAGAGA |
| 316 | TMPRSS6_83(mx) | 328 | CUCUGGUAUUUCCUAGGGUA |
| 317 | TMPRSS6_84(mx) | 774 | UUGUUACCGCUACAGCUACG |
| 318 | TMPRSS6_85(mx) | 2055 | GUCCUUCAAGGUGAGCCGCC |
| 319 | TMPRSS6_86(mx) | 334 | UAUUUCCUAGGGUACAAGGC |
| 320 | TMPRSS6_87(mx) | 333 | GUAUUUCCUAGGGUACAAGG |
| 321 | TMPRSS6_88(mx) | 843 | CAGCUGCCUGUGGCACCUGC |
| 322 | TMPRSS6_89(mx) | 2971 | UGGUCUAACUUGGGAUCUGG |
| 323 | TMPRSS6_90(mx) | 1567 | GAUGAGAGAAACUGCGUUUG |
| 324 | TMPRSS6_91(mx) | 2024 | UGUGGCAGAACUCGCGCUGG |
| 325 | TMPRSS6_92(mx) | 3165 | UGCCCUUUGGAAUAAAGCUG |
| 326 | TMPRSS6_93(mx) | 321 | GGUGCUACUCUGGUAUUUCC |
| 327 | TMPRSS6_94(mx) | 3154 | GUGAGCUCAGCUGCCCUUUG |
| 328 | TMPRSS6_95(mx) | 928 | CUGGCCAUGUAUGACGUGGC |
| 329 | TMPRSS6_96(mx) | 526 | AGCUCCGUCUAUUCCUUUGG |
| 330 | TMPRSS6_97(mx) | 1927 | GCCCUCAUCGCUGACCGCUG |
| 331 | TMPRSS6_98(mx) | 737 | GUGUGAAAGACAUAGCUGCA |
| 332 | TMPRSS6_99(mx) | 3166 | GCCCUUUGGAAUAAAGCUGC |
| 333 | TMPRSS6_100(mx) | 1243 | UACGGCUUGGCCCUCUGGUU |
| 334 | TMPRSS6_101(mx) | 1280 | GGAGGCAGAAGUAUGAUUUG |
| 335 | TMPRSS6_102(mx) | 2541 | GGUGACCUGAGGAACUGCCC |
| 336 | TMPRSS6_103(mx) | 446 | UCCGCAGUGAAACCGCCAAA |
| 337 | TMPRSS6_104(mx) | 738 | UGUGAAAGACAUAGCUGCAU |
| 338 | TMPRSS6_105(mx) | 451 | AGUGAAACCGCCAAAGCCCA |
| 339 | TMPRSS6_106(mx) | 1707 | UGGGACAUUCACCUUCCAGU |
| 340 | TMPRSS6_107(mx) | 2849 | UGGCUCAGCAGCAAGAAUGC |
| 341 | TMPRSS6_108(mx) | 638 | UGGUGGAGGAGCUGCUGUCC |
| 342 | TMPRSS6_109(mx) | 1044 | CGUCUGGAAGAAGGGCCUGC |
| 343 | TMPRSS6_110(mx) | 2851 | GCUCAGCAGCAAGAAUGCUG |
| 344 | TMPRSS6_111(mx) | 520 | AACUCCAGCUCCGUCUAUUC |
| 345 | TMPRSS6_112(mx) | 1060 | CUGCACAGCUACUACGACCC |
| 346 | TMPRSS6_113(mx) | 2903 | CCCACUCUGUACAGAGGCUG |
| 347 | TMPRSS6_114(mx) | 734 | CCAGUGUGAAAGACAUAGCU |
| 348 | TMPRSS6_115(mx) | 413 | CCCAGGAUCUUACCCGCCGG |
| 349 | TMPRSS6_116(mx) | 1945 | UGGGUGAUAACAGCUGCCCA |
| 350 | TMPRSS6_117(mx) | 1568 | AUGAGAGAAACUGCGUUUGC |
| 351 | TMPRSS6_118(mx) | 2494 | GGCGUCUACACCCGCAUCAC |
| 352 | TMPRSS6_119(mx) | 1099 | CCGGUGGUCUUCCAGGCCUG |
| 353 | TMPRSS6_120(mx) | 736 | AGUGUGAAAGACAUAGCUGC |
| 354 | TMPRSS6_121(mx) | 3167 | CCCUUUGGAAUAAAGCUGCC |
| 355 | TMPRSS6_122(mx) | 1281 | GAGGCAGAAGUAUGAUUUGC |
| 356 | TMPRSS6_123(mx) | 2039 | GCUGGCCUGGAGAGGUGUCC |
| 357 | TMPRSS6_124(mx) | 523 | UCCAGCUCCGUCUAUUCCUU |
| 358 | TMPRSS6_125(mx) | 1582 | GUUUGCAGAGCCACAUUCCA |
| 359 | TMPRSS6_126(mx) | 2527 | UGGAUCCAGCAAGUGGUGAC |
| 360 | TMPRSS6_127(mx) | 1446 | UGUGCGGGUGCACUAUGGCU |
| 361 | TMPRSS6_128(mx) | 830 | ACCACCUGGCCUCCAGCUGC |
| 362 | TMPRSS6_129(mx) | 2697 | GAUGUCUGCUCCAGUGAUGG |
| 363 | TMPRSS6_130(mx) | 1944 | CUGGGUGAUAACAGCUGCCC |
| 364 | TMPRSS6_131(mx) | 1310 | AGGGCCAGUGGACGAUCCAG |
| 365 | TMPRSS6_132(mx) | 234 | UGAGGACUCCAAGAGAAAAG |
| 366 | TMPRSS6_133(mx) | 890 | UCCGGCUGGAGUGGACGCUG |
| 367 | TMPRSS6_134(mx) | 320 | GGGUGCUACUCUGGUAUUUC |
| 368 | TMPRSS6_135(mx) | 2353 | UGUGCCGGCUACCGCAAGGG |
| 369 | TMPRSS6_136(mx) | 2492 | UCGGCGUCUACACCCGCAUC |
| 370 | TMPRSS6_137(mx) | 448 | CGCAGUGAAACCGCCAAAGC |
| 371 | TMPRSS6_138(mx) | 729 | GGAAGCCAGUGUGAAAGACA |
| 372 | TMPRSS6_139(mx) | 3155 | UGAGCUCAGCUGCCCUUUGG |
| 373 | TMPRSS6_140(mx) | 2532 | CCAGCAAGUGGUGACCUGAG |
| 374 | TMPRSS6_141(mx) | 1564 | CUGGAUGAGAGAAACUGCGU |
| 375 | TMPRSS6_142(mx) | 654 | GUCCACAGUCAACAGCUCGG |
| 376 | TMPRSS6_143(mx) | 2475 | UGGCCGGCCUAACUACUUCG |
| 377 | TMPRSS6_144(mx) | 2041 | UGGCCUGGAGAGGUGUCCUU |
| 378 | TMPRSS6_145(mx) | 733 | GCCAGUGUGAAAGACAUAGC |
| 379 | TMPRSS6_146(mx) | 2501 | ACACCCGCAUCACAGGUGUG |
| 380 | TMPRSS6_147(mx) | 1566 | GGAUGAGAGAAACUGCGUUU |
| 381 | TMPRSS6_148(mx) | 2295 | GAUCCCACAGGACCUGUGCA |
| 382 | TMPRSS6_149(mx) | 2531 | UCCAGCAAGUGGUGACCUGA |
| 383 | TMPRSS6_150(mx) | 235 | GAGGACUCCAAGAGAAAAGC |
| 384 | TMPRSS6_151(mx) | 956 | UGGAGAAGAGGCUCAUCACC |
| 385 | TMPRSS6_152(mx) | 2040 | CUGGCCUGGAGAGGUGUCCU |
| 386 | TMPRSS6_153(mx) | 571 | UGGUUCAUUCUCCAAAUCCC |
| 387 | TMPRSS6_154(mx) | 1444 | GGUGUGCGGGUGCACUAUGG |
| 388 | TMPRSS6_155(mx) | 2979 | CUUGGGAUCUGGGAAUGGAA |
| 389 | TMPRSS6_156(mx) | 735 | CAGUGUGAAAGACAUAGCUG |
| 390 | TMPRSS6_157(mx) | 2660 | CCUGUGGUGGCAGGAGGUGG |
| 391 | TMPRSS6_158(mx) | 1319 | GGACGAUCCAGAACAGGAGG |
| 392 | TMPRSS6_159(mx) | 2970 | CUGGUCUAACUUGGGAUCUG |
| 393 | TMPRSS6_160(mx) | 1738 | AGCUGCGUGAAGAAGCCCAA |
| 394 | TMPRSS6_161(mx) | 1282 | AGGCAGAAGUAUGAUUUGCC |
| 395 | TMPRSS6_162(mx) | 409 | UUCUCCCAGGAUCUUACCCG |
| 396 | TMPRSS6_163(mx) | 1227 | GGUGCCCUCUCUGGACUACG |
| 397 | TMPRSS6_164(mx) | 691 | GCCGAGUACGAAGUGGACCC |
| 398 | TMPRSS6_165(mx) | 2669 | GCAGGAGGUGGCAUCUUGUC |
| 399 | TMPRSS6_166(mx) | 322 | GUGCUACUCUGGUAUUUCCU |
| 400 | TMPRSS6_167(mx) | 765 | CACGCUGGGUUGUUACCGCU |
| 401 | TMPRSS6_168(mx) | 3151 | GAGGUGAGCUCAGCUGCCCU |
| 402 | TMPRSS6_169(mx) | 709 | CCCGAGGGCCUAGUGAUCCU |
| 403 | TMPRSS6_170(mx) | 2334 | GGUGACGCCACGCAUGCUGU |
| 404 | TMPRSS6_171(mx) | 1565 | UGGAUGAGAGAAACUGCGUU |
| 405 | TMPRSS6_172(mx) | 2848 | GUGGCUCAGCAGCAAGAAUG |
| 406 | TMPRSS6_173(mx) | 1297 | UUGCCGUGCACCCAGGGCCA |
| 407 | TMPRSS6_174(mx) | 2025 | GUGGCAGAACUCGCGCUGGC |
| 408 | TMPRSS6_175(mx) | 682 | CCCUACAGGGCCGAGUACGA |
| 409 | TMPRSS6_176(mx) | 2524 | AGCUGGAUCCAGCAAGUGGU |
| 410 | TMPRSS6_177(mx) | 2286 | UGUGCAGUUGAUCCCACAGG |
| 411 | TMPRSS6_178(mx) | 2288 | UGCAGUUGAUCCCACAGGAC |
| 412 | TMPRSS6_179(mx) | 1942 | CGCUGGGUGAUAACAGCUGC |
| 413 | TMPRSS6_180(mx) | 2972 | GGUCUAACUUGGGAUCUGGG |
| 414 | TMPRSS6_181(mx) | 2477 | GCCGGCCUAACUACUUCGGC |
| 415 | TMPRSS6_182(mx) | 2485 | AACUACUUCGGCGUCUACAC |
| 416 | TMPRSS6_183(mx) | 2495 | GCGUCUACACCCGCAUCACA |
| 417 | TMPRSS6_184(mx) | 339 | CCUAGGGUACAAGGCGGAGG |
| 418 | TMPRSS6_185(mx) | 1311 | GGGCCAGUGGACGAUCCAGA |
| 419 | TMPRSS6_186(mx) | 1216 | UGGCACCUCACGGUGCCCUC |
| 420 | TMPRSS6_187(mx) | 2482 | CCUAACUACUUCGGCGUCUA |
| 421 | TMPRSS6_188(mx) | 524 | CCAGCUCCGUCUAUUCCUUU |
| 422 | TMPRSS6_189(mx) | 2850 | GGCUCAGCAGCAAGAAUGCU |
| 423 | TMPRSS6_190(mx) | 2009 | UGUUCCUGGGCAAGGUGUGG |
| 424 | TMPRSS6_191(mx) | 957 | GGAGAAGAGGCUCAUCACCU |
| 425 | TMPRSS6_192(mx) | 2668 | GGCAGGAGGUGGCAUCUUGU |
| 426 | TMPRSS6_193(mx) | 769 | CUGGGUUGUUACCGCUACAG |
| 427 | TMPRSS6_194(mx) | 2321 | UCUAUCGCUACCAGGUGACG |
| 428 | TMPRSS6_195(mx) | 730 | GAAGCCAGUGUGAAAGACAU |
| 429 | TMPRSS6_196(mx) | 2337 | GACGCCACGCAUGCUGUGUG |
| 430 | TMPRSS6_197(mx) | 2588 | ACUCAGAGAGCCCAGGGCAA |
| 431 | TMPRSS6_198(mx) | 964 | AGGCUCAUCACCUCGGUGUA |
| 432 | TMPRSS6_199(mx) | 1303 | UGCACCCAGGGCCAGUGGAC |
| 433 | TMPRSS6_200(mx) | 341 | UAGGGUACAAGGCGGAGGUG |

The last position at the 3′ end in each of the constructs may be replaced by an “A”.

Table 6c shows TMPRSS6-targeting antisense sequences (i.e. first nucleic acid portion) including sugar modification information.

| | | Target | |
|---|---|---|---|
| | | posi- | |
| | | tion | |
| | Experi- | in | |
| SEQ | mental | TMPRS | |
| ID | Deno- | S6 | |
| NO: | tation | mRNA | Antisense Sequence (19mer) |
| 434 | TMPRSS6_1 | 1930 | [5Phos][mU][Ps][fC][Ps][mC][fC][mA][fG][mC][fG][mG][fU][mC][fA][mG][fC][mG] |
| | (mx) | | [Ps][fA][Ps][mU][Ps][fG][Ps][mA] |
| 435 | TMPRSS6_2 | 1971 | [5Phos][mU][Ps][fG][Ps][mG][fC][mC][fA][mU][fG][mC][fU][mG][fU][mC][fC][mU] |
| | (mx) | | [Ps][fC][Ps][mC][Ps][fU][Ps][mG] |
| 436 | TMPRSS6_3 | 2053 | [5Phos][mU][Ps][fG][Ps][mG][fC][mU][fC][mA][fC][mC][fU][mU][fG][mA][fA][mG] |
| | (mx) | | [Ps][fG][Ps][mA][Ps][fC][Ps][mA] |
| 437 | TMPRSS6_4 | 1970 | [5Phos][mU][Ps][fG][Ps][mC][fC][mA][fU][mG][fC][mU][fG][mU][fC][mC][fU][mC] |
| | (mx) | | [Ps][fC][Ps][mU][Ps][fG][Ps][mG] |
| 438 | TMPRSS6_5 | 1810 | [5Phos][mU][Ps][fC][Ps][mC][fU][mG][fG][mA][fG][mG][fC][mC][fA][mC][fA][mG] |
| | (mx) | | [Ps][fU][Ps][mC][Ps][fA][Ps][mC] |
| 439 | TMPRSS6_6 | 1931 | [5Phos][mU][Ps][fA][Ps][mC][fC][mC][fA][mG][fC][mG][fG][mU][fC][mA][fG][mC] |
| | (mx) | | [Ps][fG][Ps][mA][Ps][fU][Ps][mG] |
| 440 | TMPRSS6_7 | 1972 | [5Phos][mU][Ps][fA][Ps][mG][fG][mC][fC][mA][fU][mG][fC][mU][fG][mU][fC][mC] |
| | (mx) | | [Ps][fU][Ps][mC][Ps][fC][Ps][mU] |
| 441 | TMPRSS6_8 | 2049 | [5Phos][mU][Ps][fC][Ps][mA][fC][mC][fU][mU][fG][mA][fA][mG][fG][mA][fC][mA] |
| | (mx) | | [Ps][fC][Ps][mC][Ps][fU][Ps][mC] |
| 442 | TMPRSS6_9 | 330 | [5Phos][mU][Ps][fG][Ps][mU][fA][mC][fC][mC][fU][mA][fG][mG][fA][mA][fA][mU] |
| | (mx) | | [Ps][fA][Ps][mC][Ps][fC][Ps][mA] |
| 443 | TMPRSS6_10 | 1418 | [5Phos][mU][Ps][fG][Ps][mU][fG][mA][fG][mG][fG][mA][fG][mA][fU][mC][fU][mG] |
| | (mx) | | [Ps][fG][Ps][mG][Ps][fA][Ps][mG] |
| 444 | TMPRSS6_11 | 1413 | [5Phos][mU][Ps][fG][Ps][mG][fA][mG][fA][mU][fC][mU][fG][mG][fG][mA][fG][mG] |
| | (mx) | | [Ps][fU][Ps][mG][Ps][fA][Ps][mA] |
| 445 | TMPRSS6_12 | 1929 | [5Phos][mU][Ps][fC][Ps][mC][fA][mG][fC][mG][fG][mU][fC][mA][fG][mC][fG][mA] |
| | (mx) | | [Ps][fU][Ps][mG][Ps][fA][Ps][mG] |
| 446 | TMPRSS6_13 | 1322 | [5Phos][mU][Ps][fA][Ps][mG][fC][mC][fU][mC][fC][mU][fG][mU][fU][mC][fU][mG] |
| | (mx) | | [Ps][fG][Ps][mA][Ps][fU][Ps][mC] |
| 447 | TMPRSS6_14 | 1967 | [5Phos][mU][Ps][fA][Ps][mU][fG][mC][fU][mG][fU][mC][fC][mU][fC][mC][fU][mG] |
| | (mx) | | [Ps][fG][Ps][mA][Ps][fA][Ps][mG] |
| 448 | TMPRSS6_15 | 1415 | [5Phos][mU][Ps][fA][Ps][mG][fG][mG][fA][mG][fA][mU][fC][mU][fG][mG][fG][mA] |
| | (mx) | | [Ps][fG][Ps][mG][Ps][fU][Ps][mG] |
| 449 | TMPRSS6_16 | 1968 | [5Phos][mU][Ps][fC][Ps][mA][fU][mG][fC][mU][fG][mU][fC][mC][fU][mC][fC][mU] |
| | (mx) | | [Ps][fG][Ps][mG][Ps][fA][Ps][mA] |
| 450 | TMPRSS6_17 | 2052 | [5Phos][mU][Ps][fG][Ps][mC][fU][mC][fA][mC][fC][mU][fU][mG][fA][mA][fG][mG] |
| | (mx) | | [Ps][fA][Ps][mC][Ps][fA][Ps][mC] |
| 451 | TMPRSS6_18 | 1966 | [5Phos][mU][Ps][fU][Ps][mG][fC][mU][fG][mU][fC][mC][fU][mC][fC][mU][fG][mG] |
| | (mx) | | [Ps][fA][Ps][mA][Ps][fG][Ps][mC] |
| 452 | TMPRSS6_19 | 1969 | [5Phos][mU][Ps][fC][Ps][mC][fA][mU][fG][mC][fU][mG][fU][mC][fC][mU][fC][mC] |
| | (mx) | | [Ps][fU][Ps][mG][Ps][fG][Ps][mA] |
| 453 | TMPRSS6_20 | 1416 | [5Phos][mU][Ps][fG][Ps][mA][fG][mG][fG][mA][fG][mA][fU][mC][fU][mG][fG][mG] |
| | (mx) | | [Ps][fA][Ps][mG][Ps][fG][Ps][mU] |
| 454 | TMPRSS6_21 | 331 | [5Phos][mU][Ps][fU][Ps][mG][fU][mA][fC][mC][fC][mU][fA][mG][fG][mA][fA][mA] |
| | (mx) | | [Ps][fU][Ps][mA][Ps][fC][Ps][mC] |
| 455 | TMPRSS6_22 | 332 | [5Phos][mU][Ps][fU][Ps][mU][fG][mU][fA][mC][fC][mC][fU][mA][fG][mG][fA][mA] |
| | (mx) | | [Ps][fA][Ps][mU][Ps][fA][Ps][mC] |
| 456 | TMPRSS6_23 | 1808 | [5Phos][mU][Ps][fU][Ps][mG][fG][mA][fG][mG][fC][mC][fA][mC][fA][mG][fU][mC] |
| | (mx) | | [Ps][fA][Ps][mC][Ps][fA][Ps][mG] |
| 457 | TMPRSS6_24 | 1974 | [5Phos][mU][Ps][fG][Ps][mG][fA][mG][fG][mC][fC][mA][fU][mG][fC][mU][fG][mU] |
| | (mx) | | [Ps][fC][Ps][mC][Ps][fU][Ps][mC] |
| 458 | TMPRSS6_25 | 2050 | [5Phos][mU][Ps][fU][Ps][mC][fA][mC][fC][mU][fU][mG][fA][mA][fG][mG][fA][mC] |
| | (mx) | | [Ps][fA][Ps][mC][Ps][fC][Ps][mU] |
| 459 | TMPRSS6_26 | 2051 | [5Phos][mU][Ps][fC][Ps][mU][fC][mA][fC][mC][fU][mU][fG][mA][fA][mG][fG][mA] |
| | (mx) | | [Ps][fC][Ps][mA][Ps][fC][Ps][mC] |
| 460 | TMPRSS6_27 | 1807 | [5Phos][mU][Ps][fG][Ps][mG][fA][mG][fG][mC][fC][mA][fC][mA][fG][mU][fC][mA] |
| | (mx) | | [Ps][fC][Ps][mA][Ps][fG][Ps][mU] |
| 461 | TMPRSS6_28 | 1965 | [5Phos][mU][Ps][fG][Ps][mC][fU][mG][fU][mC][fC][mU][fC][mC][fU][mG][fG][mA] |
| | (mx) | | [Ps][fA][Ps][mG][Ps][fC][Ps][mA] |
| 462 | TMPRSS6_29 | 556 | [5Phos][mU][Ps][fA][Ps][mC][fC][mA][fG][mA][fA][mG][fA][mA][fG][mC][fA][mG] |
| | (mx) | | [Ps][fG][Ps][mU][Ps][fG][Ps][mA] |
| 463 | TMPRSS6_30 | 1325 | [5Phos][mU][Ps][fC][Ps][mA][fC][mA][fG][mC][fC][mU][fC][mC][fU][mG][fU][mU] |
| | (mx) | | [Ps][fC][Ps][mU][Ps][fG][Ps][mG] |
| 464 | TMPRSS6_31 | 1327 | [5Phos][mU][Ps][fC][Ps][mA][fC][mA][fC][mA][fG][mC][fC][mU][fC][mC][fU][mG] |
| | (mx) | | [Ps][fU][Ps][mU][Ps][fC][Ps][mU] |
| 465 | TMPRSS6_32 | 1562 | [5Phos][mU][Ps][fC][Ps][mA][fG][mU][fU][mU][C][mU][fC][mU][fC][mA][fU][mC] |
| | (mx) | | [Ps][fC][Ps][mA][Ps][fG][Ps][mG] |
| 466 | TMPRSS6_33 | 1234 | [5Phos][mU][Ps][fC][Ps][mC][fA][mA][fG][mC][fC][mG][fU][mA][fG][mU][fC][mC] |
| | (mx) | | [Ps][fA][Ps][mG][Ps][fA][Ps][mG] |
| 467 | TMPRSS6_34 | 557 | [5Phos][mU][Ps][fA][Ps][mA][fC][mC][fA][mG][fA][mA][fG][mA][fA][mG][IC][mA] |
| | (mx) | | [Ps][fG][Ps][mG][Ps][fU][Ps][mG] |
| 468 | TMPRSS6_35 | 555 | [5Phos][mU][Ps][fC][Ps][mC][fA][mG][fA][mA][fG][mA][fA][mG][fC][mA][fG][mG] |
| | (mx) | | [Ps][fU][Ps][mG][Ps][fA][Ps][mG] |
| 469 | TMPRSS6_36 | 460 | [5Phos][mU][Ps][fG][Ps][mC][fA][mU][fC][mU][fU][mC][fU][mG][fG][mG][fC][mU] |
| | (mx) | | [Ps][fU][Ps][mU][Ps][fG][Ps][mG] |
| 470 | TMPRSS6_37 | 1233 | [5Phos][mU][Ps][fC][Ps][mA][fA][mG][fC][mC][fG][mU][fA][mG][fU][mC][fC][mA] |
| | (mx) | | [Ps][fG][Ps][mA][Ps][fG][Ps][mA] |
| 471 | TMPRSS6_38 | 1861 | [5Phos][mU][Ps][fG][Ps][mC][fC][mA][fC][mU][fC][mA][fC][mC][fC][mU][fC][mG] |
| | (mx) | | [Ps][fG][Ps][mA][Ps][fG][Ps][mG] |
| 472 | TMPRSS6_39 | 1326 | [5Phos][mU][Ps][fA][Ps][mC][fA][mC][fA][mG][fC][mC][fU][mC][fC][mU][fG][mU] |
| | (mx) | | [Ps][fU][Ps][mC][Ps][fU][Ps][mG] |
| 473 | TMPRSS6_40 | 1235 | [5Phos][mU][Ps][fG][Ps][mC][fC][mA][fA][mG][fC][mC][fG][mU][fA][mG][fU][mC] |
| | (mx) | | [Ps][fC][Ps][mA][Ps][fG][Ps][mA] |
| 474 | TMPRSS6_41 | 2426 | [5Phos][mU][Ps][fA][Ps][mG][fG][mA][fA][mC][fC][mA][fG][mC][fG][mG][fC][mC] |
| | (mx) | | [Ps][fA][Ps][mC][Ps][fU][Ps][mG] |
| 475 | TMPRSS6_42 | 1857 | [5Phos][mU][Ps][fC][Ps][mU][fC][mA][fC][mC][fC][mU][fC][mG][fG][mA][fG][mG] |
| | (mx) | | [Ps][fA][Ps][mC][Ps][fA][Ps][mC] |
| 476 | TMPRSS6_43 | 1858 | [5Phos][mU][Ps][fA][Ps][mC][fU][mC][fA][mC][fC][mC][fU][mC][fG][mG][fA][mG] |
| | (mx) | | [Ps][fG][Ps][mA][Ps][fC][Ps][mA] |
| 477 | TMPRSS6_44 | 461 | [5Phos][mU][Ps][fA][Ps][mG][fC][mA][fU][mC][fU][mU][fC][mU][fG][mG][G][mC] |
| | (mx) | | [Ps][fU][Ps][mU][Ps][fU][Ps][mG] |
| 478 | TMPRSS6_45 | 1859 | [5Phos][mU][Ps][fC][Ps][mA][fC][mU][fC][mA][fC][mC][fC][mU][fC][mG][fG][mA] |
| | (mx) | | [Ps][fG][Ps][mG][Ps][fA][Ps][mC] |
| 479 | TMPRSS6_46 | 2027 | [5Phos][mU][Ps][fG][Ps][mG][fC][mC][fA][mG][fC][mG][fC][mG][fA][mG][fU][mU] |
| | (mx) | | [Ps][fC][Ps][mU][Ps][fG][Ps][mC] |
| 480 | TMPRSS6_47 | 462 | [5Phos][mU][Ps][fG][Ps][mA][fG][mC][fA][mU][fC][mU][fU][mC][fU][mG][fG][mG] |
| | (mx) | | [Ps][fC][Ps][mU][Ps][fU][Ps][mU] |
| 481 | TMPRSS6_48 | 1862 | [5Phos][mU][Ps][fG][Ps][mG][fC][mC][fA][mC][fU][mC][fA][mC][fC][mC][fU][mC] |
| | (mx) | | [Ps][fG][Ps][mG][Ps][fA][Ps][mG] |
| 482 | TMPRSS6_49 | 1324 | [5Phos][mU][Ps][fA][Ps][mC][fA][mG][fC][mC][fU][mC][fC][mU][fG][mU][fU][mC] |
| | (mx) | | [Ps][fU][Ps][mG][Ps][fG][Ps][mA] |
| 483 | TMPRSS6_50 | 1561 | [5Phos][mU][Ps][fA][Ps][mG][fU][mU][fU][mC][fU][mC][fU][mC][fA][mU][fC][mC] |
| | (mx) | | [Ps][fA][Ps][mG][Ps][fG][Ps][mC] |
| 484 | TMPRSS6_51 | 1560 | [5Phos][mU][Ps][fG][Ps][mU][fU][mU][fC][mU][fC][mU][fC][mA][fU][mC][IC][mA] |
| | (mx) | | [Ps][fG][Ps][mG][Ps][fC][Ps][mC] |
| 485 | TMPRSS6_52 | 1323 | [5Phos][mU][Ps][fC][Ps][mA][fG][mC][fC][mU][fC][mC][fU][mG][fU][mU][fC][mU] |
| | (mx) | | [Ps][fG][Ps][mG][Ps][fA][Ps][mU] |
| 486 | TMPRSS6_53 | 1866 | [5Phos][mU][Ps][fC][Ps][mC][fA][mU][fG][mG][fC][mC][fA][mC][fU][mC][fA][mC] |
| | (mx) | | [Ps][fC][Ps][mC][Ps][fU][Ps][mC] |
| 487 | TMPRSS6_54 | 1867 | [5Phos][mU][Ps][fG][Ps][mC][fC][mA][fU][mG][fG][mC][fC][mA][fC][mU][fC][mA] |
| | (mx) | | [Ps][fC][Ps][mC][Ps][fC][Ps][mU] |
| 488 | TMPRSS6_55 | 2358 | [5Phos][mU][Ps][fC][Ps][mU][fU][mG][fC][mC][fC][mU][fU][mG][fC][mG][fG][mU] |
| | (mx) | | [Ps][fA][Ps][mG][Ps][fC][Ps][mC] |
| 489 | TMPRSS6_56 | 2360 | [5Phos][mU][Ps][fU][Ps][mU][fC][mU][fU][mG][fC][mC][fC][mU][fU][mG][fC][mG] |
| | (mx) | | [Ps][fG][Ps][mU][Ps][fA][Ps][mG] |
| 490 | TMPRSS6_57 | 1231 | [5Phos][mU][Ps][fA][Ps][mG][fC][mC][fG][mU][fA][mG][fU][mC][fC][mA][fG][mA] |
| | (mx) | | [Ps][fG][Ps][mA][Ps][fG][Ps][mG] |
| 491 | TMPRSS6_58 | 1865 | [5Phos][mU][Ps][fC][Ps][mA][fU][mG][fG][mC][fC][mA][fC][mU][C][mA][fC][mC] |
| | (mx) | | [Ps][fC][Ps][mU][Ps][fC][Ps][mG] |
| 492 | TMPRSS6_59 | 1328 | [5Phos][mU][Ps][fC][Ps][mC][fA][mC][fA][mC][fA][mG][fC][mC][fU][mC][fC][mU] |
| | (mx) | | [Ps][fG][Ps][mU][Ps][fU][Ps][mC] |
| 493 | TMPRSS6_60 | 1863 | [5Phos][mU][Ps][fU][Ps][mG][fG][mC][fC][mA][fC][mU][fC][mA][fC][mC][IC][mU] |
| | (mx) | | [Ps][fC][Ps][mG][Ps][fG][Ps][mA] |
| 494 | TMPRSS6_61 | 1230 | [5Phos][mU][Ps][fG][Ps][mC][fC][mG][fU][mA][fG][mU][fC][mC][fA][mG][fA][mG] |
| | (mx) | | [Ps][fA][Ps][mG][Ps][fG][Ps][mG] |
| 495 | TMPRSS6_62 | 1860 | [5Phos][mU][Ps][fC][Ps][mC][fA][mC][fU][mC][fA][mC][fC][mC][fU][mC][fG][mG] |
| | (mx) | | [Ps][fA][Ps][mG][Ps][fG][Ps][mA] |
| 496 | TMPRSS6_63 | 1868 | [5Phos][mU][Ps][fU][Ps][mG][fC][mC][fA][mU][fG][mG][fC][mC][fA][mC][fU][mC] |
| | (mx) | | [Ps][fA][Ps][mC][Ps][fC][Ps][mC] |
| 497 | TMPRSS6_64 | 2359 | [5Phos][mU][Ps][fU][Ps][mC][fU][mU][fG][mC][fC][mC][fU][mU][fG][mC][fG][mG] |
| | (mx) | | [Ps][fU][Ps][mA][Ps][fG][Ps][mC] |
| 498 | TMPRSS6_65 | 1484 | [5Phos][mU][Ps][fA][Ps][mG][fG][mA][fA][mC][fU][mC][fU][mC][fC][mA][fG][mG] |
| | (mx) | | [Ps][fG][Ps][mC][Ps][fA][Ps][mG] |
| 499 | TMPRSS6_66 | 329 | [5Phos][mU][Ps][fU][Ps][mA][fC][mC][fC][mU][fA][mG][fG][mA][fA][mA][fU][mA] |
| | (mx) | | [Ps][fC][Ps][mC][Ps][fA][Ps][mG] |
| 500 | TMPRSS6_67 | 1805 | [5Phos][mU][Ps][fA][Ps][mG][fG][mC][fC][mA][fC][mA][fG][mU][fC][mA][fC][mA] |
| | (mx) | | [Ps][fG][Ps][mU][Ps][fG][Ps][mC] |
| 501 | TMPRSS6_68 | 338 | [5Phos][mU][Ps][fU][Ps][mC][fC][mG][fC][mC][fU][mU][fG][mU][fA][mC][fC][mC] |
| | (mx) | | [Ps][fU][Ps][mA][Ps][fG][Ps][mG] |
| 502 | TMPRSS6_69 | 2057 | [5Phos][mU][Ps][fA][Ps][mG][fG][mC][fG][mG][fC][mU][fC][mA][fC][mC][fU][mU] |
| | (mx) | | [Ps][fG][Ps][mA][Ps][fA][Ps][mG] |
| 503 | TMPRSS6_70 | 1485 | [5Phos][mU][Ps][fG][Ps][mA][fG][mG][fA][mA][fC][mU][fC][mU][fC][mC][fA][mG] |
| | (mx) | | [Ps][fG][Ps][mG][Ps][fC][Ps][mA] |
| 504 | TMPRSS6_71 | 1555 | [5Phos][mU][Ps][fU][Ps][mC][fU][mC][fA][mU][fC][mC][fA][mG][fG][mC][fC][mG] |
| | (mx) | | [Ps][fU][Ps][mU][Ps][fG][Ps][mG] |
| 505 | TMPRSS6_72 | 337 | [5Phos][mU][Ps][fC][Ps][mC][fG][mC][C][mU][fU][mG][fU][mA][fC][mC][fC][mU] |
| | (mx) | | [Ps][fA][Ps][mG][Ps][fG][Ps][mA] |
| 506 | TMPRSS6_73 | 777 | [5Phos][mU][Ps][fC][Ps][mA][fC][mG][fU][mA][fG][mC][fU][mG][fU][mA][fG][mC] |
| | (mx) | | [Ps][fG][Ps][mG][Ps][fU][Ps][mA] |
| 507 | TMPRSS6_74 | 2056 | [5Phos][mU][Ps][fG][Ps][mG][fC][mG][fG][mC][fU][mC][fA][mC][fC][mU][fU][mG] |
| | (mx) | | [Ps][fA][Ps][mA][Ps][fG][Ps][mG] |
| 508 | TMPRSS6_75 | 560 | [5Phos][mU][Ps][fA][Ps][mU][fG][mA][fA][mC][fC][mA][G][mA][fA][mG][fA][mA] |
| | (mx) | | [Ps][fG][Ps][mC][Ps][fA][Ps][mG] |
| 509 | TMPRSS6_76 | 327 | [5Phos][mU][Ps][fC][Ps][mC][fC][mU][fA][mG][fG][mA][fA][mA][fU][mA][fC][C] |
| | (mx) | | [Ps][fA][Ps][mG][Ps][fA][Ps][mG] |
| 510 | TMPRSS6_77 | 775 | [5Phos][mU][Ps][fC][Ps][mG][fU][mA][fG][mC][fU][mG][fU][mA][fG][mC][fG][mG] |
| | (mx) | | [Ps][fU][Ps][mA][Ps][fA][Ps][mC] |
| 511 | TMPRSS6_78 | 335 | [5Phos][mU][Ps][fG][Ps][mC][fC][mU][fU][mG][fU][mA][fC][mC][fC][mU][fA][mG] |
| | (mx) | | [Ps][fG][Ps][mA][Ps][fA][Ps][mA] |
| 512 | TMPRSS6_79 | 1804 | [5Phos][mU][Ps][fG][Ps][mG][fC][mC][fA][mC][fA][mG][fU][mC][fA][mC][fA][mG] |
| | (mx) | | [Ps][fU][Ps][mG][Ps][fC][Ps][mU] |
| 513 | TMPRSS6_80 | 846 | [5Phos][mU][Ps][fC][Ps][mU][fG][mC][fA][mG][fG][mU][fG][mC][fC][mA][fC][mA] |
| | (mx) | | [Ps][fG][Ps][mG][Ps][fC][Ps][mA] |
| 514 | TMPRSS6_81 | 776 | [5Phos][mU][Ps][fA][Ps][mC][G][mU][fA][mG][fC][mU][fG][mU][fA][mG][fC][mG] |
| | (mx) | | [Ps][fG][Ps][mU][Ps][fA][Ps][mA] |
| 515 | TMPRSS6_82 | 1556 | [5Phos][mU][Ps][fC][Ps][mU][fC][mU][fC][mA][fU][mC][fC][mA][fG][mG][fC][mC] |
| | (mx) | | [Ps][fG][Ps][mU][Ps][fU][Ps][mG] |
| 516 | TMPRSS6_83 | 328 | [5Phos][mU][Ps][fA][Ps][mC][fC][mC][fU][mA][fG][mG][fA][mA][fA][mU][fA][mC] |
| | (mx) | | [Ps][fC][Ps][mA][Ps][fG][Ps][mA] |
| 517 | TMPRSS6_84 | 774 | [5Phos][mU][Ps][fG][Ps][mU][fA][mG][fC][mU][fG][mU][fA][mG][fC][mG][fG][mU] |
| | (mx) | | [Ps][fA][Ps][mA][Ps][fC][Ps][mA] |
| 518 | TMPRSS6_85 | 2055 | [5Phos][mU][Ps][fG][Ps][mC][fG][mG][fC][mU][fC][mA][fC][mC][fU][mU][fG][mA] |
| | (mx) | | [Ps][fA][Ps][mG][Ps][fG][Ps][mA] |
| 519 | TMPRSS6_86 | 334 | [5Phos][mU][Ps][fC][Ps][mC][fU][mU][fG][mU][fA][mC][fC][mC][fU][mA][fG][mG] |
| | (mx) | | [Ps][fA][Ps][mA][Ps][fA][Ps][mU] |
| 520 | TMPRSS6_87 | 333 | [5Phos][mU][Ps][fC][Ps][mU][fU][mG][fU][mA][fC][mC][fC][mU][fA][mG][fG][mA] |
| | (mx) | | [Ps][fA][Ps][mA][Ps][fU][Ps][mA] |
| 521 | TMPRSS6_88 | 843 | [5Phos][mU][Ps][fC][Ps][mA][fG][mG][fU][mG][fC][mC][fA][mC][fA][mG][fG][mC] |
| | (mx) | | [Ps][fA][Ps][mG][Ps][fC][Ps][mU] |
| 522 | TMPRSS6_89 | 2971 | [5Phos][mU][Ps][fC][Ps][mA][fG][mA][fU][mC][fC][mC][fA][mA][fG][mU][fU][mA] |
| | (mx) | | [Ps][fG][Ps][mA][Ps][fC][Ps][mC] |
| 523 | TMPRSS6_90 | 1567 | [5Phos][mU][Ps][fA][Ps][mA][fA][mC][fG][mC][fA][mG][fU][mU][fU][mC][fU][mC] |
| | (mx) | | [Ps][fU][Ps][mC][Ps][fA][Ps][mU] |
| 524 | TMPRSS6_91 | 2024 | [5Phos][mU][Ps][fC][Ps][mA][fG][mC][fG][mC][fG][mA][fG][mU][fU][mC][fU][mG] |
| | (mx) | | [Ps][fC][Ps][mC][Ps][fA][Ps][mC] |
| 525 | TMPRSS6_92 | 3165 | [5Phos][mU][Ps][fA][Ps][mG][fC][mU][fU][mU][fA][mU][fU][mC][fC][mA][fA][mA] |
| | (mx) | | [Ps][fG][Ps][mG][Ps][fG][Ps][mC] |
| 526 | TMPRSS6_93 | 321 | [5Phos][mU][Ps][fG][Ps][mA][fA][mA][fU][mA][fC][mC][fA][mG][fA][mG][fU][mA] |
| | (mx) | | [Ps][fG][Ps][mC][Ps][fA][Ps][mC] |
| 527 | TMPRSS6_94 | 3154 | [5Phos][mU][Ps][fA][Ps][mA][fA][mG][fG][mG][C][mA][fG][mC][fU][mG][fA][mG] |
| | (mx) | | [Ps][fC][Ps][mU][Ps][fC][Ps][mA] |
| 528 | TMPRSS6_95 | 928 | [5Phos][mU][Ps][fC][Ps][mC][fA][mC][fG][mU][fC][mA][fU][mA][fC][mA][fU][mG] |
| | (mx) | | [Ps][fG][Ps][mC][Ps][fC][Ps][mA] |
| 529 | TMPRSS6_96 | 526 | [5Phos][mU][Ps][fC][Ps][mA][fA][mA][fG][mG][fA][mA][fU][mA][fG][mA][fC][mG] |
| | (mx) | | [Ps][fG][Ps][mA][Ps][fG][Ps][mC] |
| 530 | TMPRSS6_97 | 1927 | [5Phos][mU][Ps][fA][Ps][mG][fC][mG][fG][mU][fC][mA][fG][mC][fG][mA][fU][mG] |
| | (mx) | | [Ps][fA][Ps][mG][Ps][fG][Ps][mG] |
| 531 | TMPRSS6_98 | 737 | [5Phos][mU][Ps][fG][Ps][mC][fA][mG][fC][mU][fA][mU][fG][mU][fC][mU][fU][mU] |
| | (mx) | | [Ps][fC][Ps][mA][Ps][fC][Ps][mA] |
| 532 | TMPRSS6_99 | 3166 | [5Phos][mU][Ps][fC][Ps][mA][fG][mC][fU][mU][fU][mA][fU][mU][fC][mC][fA][mA] |
| | (mx) | | [Ps][fA][Ps][mG][Ps][fG][Ps][mG] |
| 533 | TMPRSS6_100 | 1243 | [5Phos][mU][Ps][fA][Ps][mC][fC][mA][fG][mA][G][mG][fG][mC][fC][mA][fA][mG] |
| | (mx) | | [Ps][fC][Ps][mC][Ps][fG][Ps][mU] |
| 534 | TMPRSS6_101 | 1280 | [5Phos][mU][Ps][fA][Ps][mA][fA][mU][fC][mA][fU][mA][fC][mU][fU][mC][fU][mG] |
| | (mx) | | [Ps][fC][Ps][mC][Ps][fU][Ps][mC] |
| 535 | TMPRSS6_102 | 2541 | [5Phos][mU][Ps][fG][Ps][mG][fC][mA][fG][mU][fU][mC][fC][mU][fC][mA][fG][mG] |
| | (mx) | | [Ps][fU][Ps][mC][Ps][fA][Ps][mC] |
| 536 | TMPRSS6_103 | 446 | [5Phos][mU][Ps][fU][Ps][mU][fG][mG][fC][mG][fG][mU][fU][mU][fC][mA][fC][mU] |
| | (mx) | | [Ps][fG][Ps][mC][Ps][fG][Ps][mG] |
| 537 | TMPRSS6_104 | 738 | [5Phos][mU][Ps][fU][Ps][mG][fC][mA][fG][mC][fU][mA][fU][mG][fU][mC][fU][mU] |
| | (mx) | | [Ps][fU][Ps][mC][Ps][fA][Ps][mC] |
| 538 | TMPRSS6_105 | 451 | [5Phos][mU][Ps][fG][Ps][mG][fG][mC][fU][mU][fU][mG][fG][mC][fG][mG][fU][mU] |
| | (mx) | | [Ps][fU][Ps][mC][Ps][fA][Ps][mC] |
| 539 | TMPRSS6_106 | 1707 | [5Phos][mU][Ps][fC][Ps][mU][fG][mG][fA][mA][fG][mG][fU][mG][fA][mA][fU][mG] |
| | (mx) | | [Ps][fU][Ps][mC][Ps][fC][Ps][mC] |
| 540 | TMPRSS6_107 | 2849 | [5Phos][mU][Ps][fC][Ps][mA][fU][mU][fC][mU][fU][mG][fC][mU][fG][mC][fU][mG] |
| | (mx) | | [Ps][fA][Ps][mG][Ps][fC][Ps][mC] |
| 541 | TMPRSS6_108 | 638 | [5Phos][mU][Ps][fG][Ps][mA][fC][mA][fG][mC][fA][mG][fC][mU][fC][mC][fU][mC] |
| | (mx) | | [Ps][fC][Ps][mA][Ps][fC][Ps][mC] |
| 542 | TMPRSS6_109 | 1044 | [5Phos][mU][Ps][fC][Ps][mA][fG][mG][fC][mC][fC][mU][fU][mC][fU][mU][fC][mC] |
| | (mx) | | [Ps][fA][Ps][mG][Ps][fA][Ps][mC] |
| 543 | TMPRSS6_110 | 2851 | [5Phos][mU][Ps][fA][Ps][mG][fC][mA][fU][mU][fC][mU][fU][mG][fC][mU][fG][mC] |
| | (mx) | | [Ps][fU][Ps][mG][Ps][fA][Ps][mG] |
| 544 | TMPRSS6_111 | 520 | [5Phos][mU][Ps][fA][Ps][mA][fU][mA][fG][mA][fC][mG][fG][mA][fG][mC][fU][mG] |
| | (mx) | | [Ps][fG][Ps][mA][Ps][fG][Ps][mU] |
| 545 | TMPRSS6_112 | 1060 | [5Phos][mU][Ps][fG][Ps][mG][fU][mC][fG][mU][fA][mG][fU][mA][fG][mC][fU][mG] |
| | (mx) | | [Ps][fU][Ps][mG][Ps][fC][Ps][mA] |
| 546 | TMPRSS6_113 | 2903 | [5Phos][mU][Ps][fA][Ps][mG][fC][mC][fU][mC][fU][mG][fU][mA][fC][mA][fG][mA] |
| | (mx) | | [Ps][fG][Ps][mU][Ps][fG][Ps][mG] |
| 547 | TMPRSS6_114 | 734 | [5Phos][mU][Ps][fG][Ps][mC][fU][mA][fU][mG][fU][mC][fU][mU][fU][mC][fA][mC] |
| | (mx) | | [Ps][fA][Ps][mC][Ps][fU][Ps][mG] |
| 548 | TMPRSS6_115 | 413 | [5Phos][mU][Ps][fC][Ps][mG][fG][mC][fG][mG][fG][mU][fA][mA][fG][mA][fU][mC] |
| | (mx) | | [Ps][fC][Ps][mU][Ps][fG][Ps][mG] |
| 549 | TMPRSS6_116 | 1945 | [5Phos][mU][Ps][fG][Ps][mG][fG][mC][fA][mG][fC][mU][fG][mU][fU][mA][fU][mC] |
| | (mx) | | [Ps][fA][Ps][mC][Ps][fC][Ps][mC] |
| 550 | TMPRSS6_117 | 1568 | [5Phos][mU][Ps][fC][Ps][mA][fA][mA][fC][mG][fC][mA][fG][mU][fU][mU][fC][mU] |
| | (mx) | | [Ps][fC][Ps][mU][Ps][fC][Ps][mA] |
| 551 | TMPRSS6_118 | 2494 | [5Phos][mU][Ps][fU][Ps][mG][fA][mU][fG][mC][fG][mG][fG][mU][fG][mU][fA][mG] |
| | (mx) | | [Ps][fA][Ps][mC][Ps][fG][Ps][mC] |
| 552 | TMPRSS6_119 | 1099 | [5Phos][mU][Ps][fA][Ps][mG][fG][mC][fC][mU][fG][mG][fA][mA][fG][mA][fC][mC] |
| | (mx) | | [Ps][fA][Ps][mC][Ps][fC][Ps][mG] |
| 553 | TMPRSS6_120 | 736 | [5Phos][m]][Ps][fC][Ps][mA][fG][mC][fU][mA][fU][mG][fU][mC][fU][mU][fU][mC] |
| | (mx) | | [Ps][fA][Ps][mC][Ps][fA][Ps][mC] |
| 554 | TMPRSS6_121 | 3167 | [5Phos][mU][Ps][fG][Ps][mC][fA][mG][fC][mU][fU][mU][fA][mU][fU][mC][fC][mA] |
| | (mx) | | [Ps][fA][Ps][mA][Ps][fG][Ps][mG] |
| 555 | TMPRSS6_122 | 1281 | [5Phos][mU][Ps][fC][Ps][mA][fA][mA][fU][mC][fA][mU][fA][mC][fU][mU][fC][mU] |
| | (mx) | | [Ps][fG][Ps][mC][Ps][fC][Ps][mU] |
| 556 | TMPRSS6_123 | 2039 | [5Phos][mU][Ps][fG][Ps][mA][fC][mA][fC][mC][fU][mC][fU][mC][fC][mA][fG][mG] |
| | (mx) | | [Ps][fC][Ps][mC][Ps][fA][Ps][mG] |
| 557 | TMPRSS6_124 | 523 | [5Phos][mU][Ps][fA][Ps][mG][fG][mA][fA][mU][fA][mG][fA][mC][fG][mG][fA][mG] |
| | (mx) | | [Ps][fC][Ps][mU][Ps][fG][Ps][mG] |
| 558 | TMPRSS6_125 | 1582 | [5Phos][mU][Ps][fG][Ps][mG][fA][mA][fU][mG][fU][mG][fG][mC][fU][mC][fU][mG] |
| | (mx) | | [Ps][fC][Ps][mA][Ps][fA][Ps][mA] |
| 559 | TMPRSS6_126 | 2527 | [5Phos][mU][Ps][fU][Ps][mC][fA][mC][fC][mA][fC][mU][fU][mG][fC][mU][fG][mG] |
| | (mx) | | [Ps][fA][Ps][mU][Ps][fC][Ps][mC] |
| 560 | TMPRSS6_127 | 1446 | [5Phos][mU][Ps][fG][Ps][mC][fC][mA][fU][mA][fG][mU][G][mC][fA][mC][fC][mC] |
| | (mx) | | [Ps][fG][Ps][mC][Ps][fA][Ps][mC] |
| 561 | TMPRSS6_128 | 830 | [5Phos][mU][Ps][fC][Ps][mA][fG][mC][fU][mG][fG][mA][fG][mG][fC][mC][fA][mG] |
| | (mx) | | [Ps][fG][Ps][mU][Ps][fG][Ps][mG] |
| 562 | TMPRSS6_129 | 2697 | [5Phos][mU][Ps][fC][Ps][mA][fU][mC][fA][mC][fU][mG][fG][mA][fG][mC][fA][mG] |
| | (mx) | | [Ps][fA][Ps][mC][Ps][fA][Ps][mU] |
| 563 | TMPRSS6_130 | 1944 | [5Phos][mU][Ps][fG][Ps][mG][fC][mA][fG][mC][fU][mG][fU][mU][fA][mU][fC][mA] |
| | (mx) | | [Ps][fC][Ps][mC][Ps][fC][Ps][mA] |
| 564 | TMPRSS6_131 | 1310 | [5Phos][mU][Ps][fU][Ps][mG][fG][mA][fU][mC][fG][mU][fC][mC][fA][mC][fU][mG] |
| | (mx) | | [Ps][fG][Ps][mC][Ps][fC][Ps][mC] |
| 565 | TMPRSS6_132 | 234 | [5Phos][mU][Ps][fU][Ps][mU][fU][mU][fC][mU][fC][mU][fU][mG][fG][mA][fG][mU] |
| | (mx) | | [Ps][fC][Ps][mC][Ps][fU][Ps][mC] |
| 566 | TMPRSS6_133 | 890 | [5Phos][mU][Ps][fA][Ps][mG][fC][mG][fU][mC][fC][mA][fC][mU][fC][mC][fA][mG] |
| | (mx) | | [Ps][fC][Ps][mC][Ps][fG][Ps][mG] |
| 567 | TMPRSS6_134 | 320 | [5Phos][mU][Ps][fA][Ps][mA][fA][mU][fA][mC][fC][mA][G][mA][fG][mU][fA][mG] |
| | (mx) | | [Ps][fC][Ps][mA][Ps][fC][Ps][mC] |
| 568 | TMPRSS6_135 | 2353 | [5Phos][mU][Ps][fC][Ps][mC][fU][mU][fG][mC][fG][mG][fU][mA][fG][mC][fC][mG] |
| | (mx) | | [Ps][fG][Ps][mC][Ps][fA][Ps][mC] |
| 569 | TMPRSS6_136 | 2492 | [5Phos][mU][Ps][fA][Ps][mU][fG][mC][fG][mG][fG][mU][fG][mU][fA][mG][fA][mC] |
| | (mx) | | [Ps][fG][Ps][mC][Ps][fC][Ps][mG] |
| 570 | TMPRSS6_137 | 448 | [5Phos][mU][Ps][fC][Ps][mU][fU][mU][fG][mG][fC][mG][fG][mU][fU][mU][fC][mA] |
| | (mx) | | [Ps][fC][Ps][mU][Ps][fG][Ps][mC] |
| 571 | TMPRSS6_138 | 729 | [5Phos][mU][Ps][fG][Ps][mU][fC][mU][fU][mU][fC][mA][fC][mA][fC][mU][fG][mG] |
| | (mx) | | [Ps][fC][Ps][mU][Ps][fU][Ps][mC] |
| 572 | TMPRSS6_139 | 3155 | [5Phos][mU][Ps][fC][Ps][mA][fA][mA][fG][mG][fG][mC][fA][mG][fC][mU][fG][mA] |
| | (mx) | | [Ps][fG][Ps][mC][Ps][fU][Ps][mC] |
| 573 | TMPRSS6_140 | 2532 | [5Phos][mU][Ps][fU][Ps][mC][fA][mG][fG][mU][fC][mA][fC][mC][fA][mC][fU][mU] |
| | (mx) | | [Ps][fG][Ps][mC][Ps][fU][Ps][mG] |
| 574 | TMPRSS6_141 | 1564 | [5Phos][mU][Ps][fC][Ps][mG][fC][mA][fG][mU][fU][mU][fC][mU][fC][mU][fC][mA] |
| | (mx) | | [Ps][fU][Ps][mC][Ps][fC][Ps][mA] |
| 575 | TMPRSS6_142 | 654 | [5Phos][mU][Ps][fC][Ps][mG][fA][mG][fC][mU][fG][mU][fU][mG][fA][mC][fU][mG] |
| | (mx) | | [Ps][fU][Ps][mG][Ps][fG][Ps][mA] |
| 576 | TMPRSS6_143 | 2475 | [5Phos][mU][Ps][fG][Ps][mA][fA][mG][fU][mA][fG][mU][fU][mA][fG][mG][fC][mC] |
| | (mx) | | [Ps][fG][Ps][mG][Ps][fC][Ps][mC] |
| 577 | TMPRSS6_144 | 2041 | [5Phos][mU][Ps][fA][Ps][mG][fG][mA][fC][mA][fC][mC][fU][mC][fU][mC][fC][mA] |
| | (mx) | | [Ps][fG][Ps][mG][Ps][fC][Ps][mC] |
| 578 | TMPRSS6_145 | 733 | [5Phos][mU][Ps][fC][Ps][mU][fA][mU][fG][mU][fC][mU][fU][mU][fC][mA][fC][mA] |
| | (mx) | | [Ps][fC][Ps][mU][Ps][fG][Ps][mG] |
| 579 | TMPRSS6_146 | 2501 | [5Phos][mU][Ps][fA][Ps][mC][fA][mC][fC][mU][fG][mU][fG][mA][fU][mG][fC][mG] |
| | (mx) | | [Ps][fG][Ps][mG][Ps][fU][Ps][mG] |
| 580 | TMPRSS6_147 | 1566 | [5Phos][mU][Ps][fA][Ps][mA][fC][mG][fC][mA][fG][mU][fU][mU][fC][mU][fC][mU] |
| | (mx) | | [Ps][fC][Ps][mA][Ps][fU][Ps][mC] |
| 581 | TMPRSS6_148 | 2295 | [5Phos][mU][Ps][fG][Ps][mC][fA][mC][fA][mG][fG][mU][fC][mC][fU][mG][fU][mG] |
| | (mx) | | [Ps][fG][Ps][mG][Ps][fA][Ps][mU] |
| 582 | TMPRSS6_149 | 2531 | [5Phos][mU][Ps][fC][Ps][mA][fG][mG][fU][mC][fA][mC][fC][mA][fC][mU][fU][mG] |
| | (mx) | | [Ps][fC][Ps][mU][Ps][fG][Ps][mG] |
| 583 | TMPRSS6_150 | 235 | [5Phos][mU][Ps][fC][Ps][mU][fU][mU][fU][mC][fU][mC][fU][mU][fG][mG][fA][mG] |
| | (mx) | | [Ps][fU][Ps][mC][Ps][fC][Ps][mU] |
| 584 | TMPRSS6_151 | 956 | [5Phos][mU][Ps][fG][Ps][mU][fG][mA][fU][mG][fA][mG][fC][mC][fU][mC][fU][mU] |
| | (mx) | | [Ps][fC][Ps][mU][Ps][fC][Ps][mC] |
| 585 | TMPRSS6_152 | 2040 | [5Phos][mU][Ps][fG][Ps][mG][fA][mC][fA][mC][fC][mU][fC][mU][fC][mC][fA][mG] |
| | (mx) | | [Ps][fG][Ps][mC][Ps][fC][Ps][mA] |
| 586 | TMPRSS6_153 | 571 | [5Phos][mU][Ps][fG][Ps][mG][fA][mU][fU][mU][fG][mG][fA][mG][fA][mA][fU][mG] |
| | (mx) | | [Ps][fA][Ps][mA][Ps][fC][Ps][mC] |
| 587 | TMPRSS6_154 | 1444 | [5Phos][mU][Ps][fC][Ps][mA][fU][mA][fG][mU][fG][mC][fA][mC][fC][mC][fG][mC] |
| | (mx) | | [Ps][fA][Ps][mC][Ps][fA][Ps][mC] |
| 588 | TMPRSS6_155 | 2979 | [5Phos][mU][Ps][fU][Ps][mC][fC][mA][fU][mU][fC][mC][fC][mA][fG][mA][fU][mC] |
| | (mx) | | [Ps][fC][Ps][mC][Ps][fA][Ps][mA] |
| 589 | TMPRSS6_156 | 735 | [5Phos][mU][Ps][fA][Ps][mG][fC][mU][fA][mU][fG][mU][fC][mU][fU][mU][fC][mA] |
| | (mx) | | [Ps][fC][Ps][mA][Ps][fC][Ps][mU] |
| 590 | TMPRSS6_157 | 2660 | [5Phos][mU][Ps][fC][Ps][mA][fC][mC][fU][mC][fC][mU][fG][mC][fC][mA][fC][mC] |
| | (mx) | | [Ps][fA][Ps][mC][Ps][fA][Ps][mG] |
| 591 | TMPRSS6_158 | 1319 | [5Phos][mU][Ps][fC][Ps][mU][fC][mC][fU][mG][fU][mU][fC][mU][fG][mG][fA][mU] |
| | (mx) | | [Ps][fC][Ps][mG][Ps][fU][Ps][mC] |
| 592 | TMPRSS6_159 | 2970 | [5Phos][mU][Ps][fA][Ps][mG][fA][mU][fC][mC][fC][mA][fA][mG][fU][mU][fA][mG] |
| | (mx) | | [Ps][fA][Ps][mC][Ps][fC][Ps][mA] |
| 593 | TMPRSS6_160 | 1738 | [5Phos][mU][Ps][fU][Ps][mG][fG][mG][fC][mU][fU][mC][fU][mU][fC][mA][fC][mG] |
| | (mx) | | [Ps][fC][Ps][mA][Ps][fG][Ps][mC] |
| 594 | TMPRSS6_161 | 1282 | [5Phos][mU][Ps][fG][Ps][mC][fA][mA][fA][mU][fC][mA][fU][mA][fC][mU][fU][mC] |
| | (mx) | | [Ps][fU][Ps][mG][Ps][fC][Ps][mC] |
| 595 | TMPRSS6_162 | 409 | [5Phos][mU][Ps][fG][Ps][mG][fG][mU][fA][mA][fG][mA][fU][mC][fC][mU][fG][mG] |
| | (mx) | | [Ps][fG][Ps][mA][Ps][fG][Ps][mA] |
| 596 | TMPRSS6_163 | 1227 | [5Phos][mU][Ps][fG][Ps][mU][fA][mG][fU][mC][fC][mA][fG][mA][fG][mA][fG][mG] |
| | (mx) | | [Ps][fG][Ps][mC][Ps][fA][Ps][mC] |
| 597 | TMPRSS6_164 | 691 | [5Phos][mU][Ps][fG][Ps][mG][fU][mC][fC][mA][fC][m]][f]][mC][fG][mU][fA][mC] |
| | (mx) | | [Ps][fU][Ps][mC][Ps][fG][Ps][mG] |
| 598 | TMPRSS6_165 | 2669 | [5Phos][mU][Ps][fA][Ps][mC][fA][mA][fG][mA][fU][mG][fC][mC][fA][mC][fC][mU] |
| | (mx) | | [Ps][fC][Ps][mC][Ps][fU][Ps][mG] |
| 599 | TMPRSS6_166 | 322 | [5Phos][mU][Ps][fG][Ps][mG][fA][mA][fA][mU][fA][mC][fC][mA][fG][mA][fG][mU] |
| | (mx) | | [Ps][fA][Ps][mG][Ps][fC][Ps][mA] |
| 600 | TMPRSS6_167 | 765 | [5Phos][mU][Ps][fG][Ps][mC][fG][mG][fU][mA][fA][mC][fA][mA][fC][mC][fC][mA] |
| | (mx) | | [Ps][fG][Ps][mC][Ps][fG][Ps][mU] |
| 601 | TMPRSS6_168 | 3151 | [5Phos][mU][Ps][fG][Ps][mG][fG][mC][fA][mG][C][mU][fG][mA][fG][mC][fU][mC] |
| | (mx) | | [Ps][fA][Ps][mC][Ps][fC][Ps][mU] |
| 602 | TMPRSS6_169 | 709 | [5Phos][mU][Ps][fG][Ps][mG][fA][mU][fC][mA][fC][mU][fA][mG][fG][mC][fC][mC] |
| | (mx) | | [Ps][fU][Ps][mC][Ps][fG][Ps][mG] |
| 603 | TMPRSS6_170 | 2334 | [5Phos][mU][Ps][fC][Ps][mA][fG][mC][fA][mU][fG][mC][fG][mU][fG][mG][fC][mG] |
| | (mx) | | [Ps][fU][Ps][mC][Ps][fA][Ps][mC] |
| 604 | TMPRSS6_171 | 1565 | [5Phos][mU][Ps][fA][Ps][mC][fG][mC][fA][mG][fU][mU][fU][mC][fU][mC][fU][mC] |
| | (mx) | | [Ps][fA][Ps][mU][Ps][fC][Ps][mC] |
| 605 | TMPRSS6_172 | 2848 | [5Phos][mU][Ps][fA][Ps][mU][fU][mC][fU][mU][fG][mC][fU][mG][fC][mU][fG][mA] |
| | (mx) | | [Ps][fG][Ps][mC][Ps][fC][Ps][mA] |
| 606 | TMPRSS6_173 | 1297 | [5Phos][mU][Ps][fG][Ps][mG][fC][mC][fC][mU][fG][mG][fG][mU][fG][mC][fA][mC] |
| | (mx) | | [Ps][fG][Ps][mG][Ps][fC][Ps][mA] |
| 607 | TMPRSS6_174 | 2025 | [5Phos][mU][Ps][fC][Ps][mC][fA][mG][fC][mG][fC][mG][fA][mG][fU][mU][fC][mU] |
| | (mx) | | [Ps][fG][Ps][mC][Ps][fC][Ps][mA] |
| 608 | TMPRSS6_175 | 682 | [5Phos][mU][Ps][fC][Ps][mG][fU][mA][fC][mU][fC][mG][fG][mC][C][mC][fU][mG] |
| | (mx) | | [Ps][fU][Ps][mA][Ps][fG][Ps][mG] |
| 609 | TMPRSS6_176 | 2524 | [5Phos][mU][Ps][fC][Ps][mC][fA][mC][fU][mU][fG][mC][fU][mG][fG][mA][fU][mC] |
| | (mx) | | [Ps][fC][Ps][mA][Ps][fG][Ps][mC] |
| 610 | TMPRSS6_177 | 2286 | [5Phos][mU][Ps][fC][Ps][mU][fG][mU][fG][mG][fG][mA][fU][mC][fA][mA][fC][mU] |
| | (mx) | | [Ps][fG][Ps][mC][Ps][fA][Ps][mC] |
| 611 | TMPRSS6_178 | 2288 | [5Phos][mU][Ps][fU][Ps][mC][fC][mU][fG][mU][fG][mG][fG][mA][fU][mC][fA][mA] |
| | (mx) | | [Ps][fC][Ps][mU][Ps][fG][Ps][mC] |
| 612 | TMPRSS6_179 | 1942 | [5Phos][mU][Ps][fC][Ps][mA][fG][mC][fU][mG][fU][mU][fA][mU][C][mA][fC][mC] |
| | (mx) | | [Ps][fC][Ps][mA][Ps][fG][Ps][mC] |
| 613 | TMPRSS6_180 | 2972 | [5Phos][mU][Ps][fC][Ps][mC][fA][mG][fA][mU][fC][mC][fC][mA][fA][mG][fU][mU] |
| | (mx) | | [Ps][fA][Ps][mG][Ps][fA][Ps][mC] |
| 614 | TMPRSS6_181 | 2477 | [5Phos][mU][Ps][fC][Ps][mC][fG][mA][fA][mG][fU][mA][fG][mU][fU][mA][fG][mG] |
| | (mx) | | [Ps][fC][Ps][mC][Ps][fG][Ps][mG] |
| 615 | TMPRSS6_182 | 2485 | [5Phos][mU][Ps][fU][Ps][mG][fU][mA][fG][mA][fC][mG][fC][mC][fG][mA][fA][mG] |
| | (mx) | | [Ps][fU][Ps][mA][Ps][fG][Ps][mU] |
| 616 | TMPRSS6_183 | 2495 | [5Phos][mU][Ps][fG][Ps][mU][fG][mA][fU][mG][fC][mG][fG][mG][fU][mG][fU][mA] |
| | (mx) | | [Ps][fG][Ps][mA][Ps][fC][Ps][mG] |
| 617 | TMPRSS6_184 | 339 | [5Phos][mU][Ps][fC][Ps][mU][fC][mC][fG][mC][fC][mU][fU][mG][fU][mA][fC][mC] |
| | (mx) | | [Ps][fC][Ps][mU][Ps][fA][Ps][mG] |
| 618 | TMPRSS6_185 | 1311 | [5Phos][mU][Ps][fC][Ps][mU][fG][mG][fA][mU][fC][mG][fU][mC][fC][mA][fC][mU] |
| | (mx) | | [Ps][fG][Ps][mG][Ps][fC][Ps][mC] |
| 619 | TMPRSS6_186 | 1216 | [5Phos][mU][Ps][fA][Ps][mG][fG][mG][fC][mA][fC][mC][fG][mU][fG][mA][fG][mG] |
| | (mx) | | [Ps][fU][Ps][mG][Ps][fC][Ps][mC] |
| 620 | TMPRSS6_187 | 2482 | [5Phos][mU][Ps][fA][Ps][mG][fA][mC][fG][mC][fC][mG][fA][mA][fG][mU][fA][mG] |
| | (mx) | | [Ps][fU][Ps][mU][Ps][fA][Ps][mG] |
| 621 | TMPRSS6_188 | 524 | [5Phos][mU][Ps][fA][Ps][mA][fG][mG][fA][mA][fU][mA][fG][mA][fC][mG][fG][mA] |
| | (mx) | | [Ps][fG][Ps][mC][Ps][fU][Ps][mG] |
| 622 | TMPRSS6_189 | 2850 | [5Phos][mU][Ps][fG][Ps][mC][fA][mU][fU][mC][fU][mU][fG][mC][fU][mG][fC][mU] |
| | (mx) | | [Ps][fG][Ps][A][Ps][fG][Ps][mC] |
| 623 | TMPRSS6_190 | 2009 | [5Phos][mU][Ps][fC][Ps][mA][fC][mA][fC][mC][fU][mU][fG][mC][fC][mC][fA][mG] |
| | (mx) | | [Ps][fG][Ps][mA][Ps][fA][Ps][mC] |
| 624 | TMPRSS6_191 | 957 | [5Phos][mU][Ps][fG][Ps][mG][fU][mG][fA][mU][fG][mA][fG][mC][fC][mU][fC][mU] |
| | (mx) | | [Ps][fU][Ps][mC][Ps][fU][Ps][mC] |
| 625 | TMPRSS6_192 | 2668 | [5Phos][mU][Ps][fC][Ps][mA][fA][mG][fA][mU][fG][mC][fC][mA][fC][mC][fU][mC] |
| | (mx) | | [Ps][fC][Ps][mU][Ps][fG][Ps][mC] |
| 626 | TMPRSS6_193 | 769 | [5Phos][mU][Ps][fU][Ps][mG][fU][mA][fG][mC][fG][mG][fU][mA][fA][mC][fA][mA] |
| | (mx) | | [Ps][fC][Ps][mC][Ps][fC][Ps][mA] |
| 627 | TMPRSS6_194 | 2321 | [5Phos][mU][Ps][fG][Ps][mU][fC][mA][fC][mC][fU][mG][fG][mU][fA][mG][fC][mG] |
| | (mx) | | [Ps][fA][Ps][mU][Ps][fA][Ps][mG] |
| 628 | TMPRSS6_195 | 730 | [5Phos][mU][Ps][fU][Ps][mG][fU][mC][fU][mU][fU][mC][fA][mC][fA][mC][fU][mG] |
| | (mx) | | [Ps][fG][Ps][mC][Ps][fU][Ps][mU] |
| 629 | TMPRSS6_196 | 2337 | [5Phos][mU][Ps][fA][Ps][mC][fA][mC][fA][mG][fC][mA][fU][mG][fC][mG][fU][mG] |
| | (mx) | | [Ps][fG][Ps][mC][Ps][fG][Ps][mU] |
| 630 | TMPRSS6_197 | 2588 | [5Phos][mU][Ps][fU][Ps][mG][fC][mC][fC][mU][fG][mG][fG][mC][fU][mC][fU][mC] |
| | (mx) | | [Ps][fU][Ps][mG][Ps][fA][Ps][mG] |
| 631 | TMPRSS6_198 | 964 | [5Phos][mU][Ps][fA][Ps][mC][fA][mC][fC][mG][fA][mG][fG][mU][fG][mA][fU][mG] |
| | (mx) | | [Ps][fA][Ps][mG][Ps][fC][Ps][mC] |
| 632 | TMPRSS6_199 | 1303 | [5Phos][mU][Ps][fU][Ps][mC][fC][mA][fC][mU][fG][mG][fC][mC][fC][mU][fG][mG] |
| | (mx) | | [Ps][fG][Ps][mU][Ps][fG][Ps][mC] |
| 633 | TMPRSS6_200 | 341 | [5Phos][mU][Ps][fA][Ps][mC][fC][mU][fC][mC][fG][mC][fC][mU][fU][mG][fU][mA] |
| | (mx) | | [Ps][fC][Ps][mC][Ps][fC][Ps][mU] |

Note
each of the above constructs may or may not have a phosphate modification at the 5′ end group. In certain embodiments, e.g. in the case of a muRNA, the 3′ terminus of the antisense sequence may be unmodified and not carry a phosphorothioate but a phosphate.

Table 7a shows modified TMPRSS6-APOC3 muRNA constructs of the present disclosure in their double stranded form (each strand of the two strands is in a separate line for the respective SEQ ID NO).

| | Experi- | |
|---|---|---|
| SEQ | mental | |
| ID | deno- | |
| NO: | tation | muRNA construct |
| 634 | TMP RSS | [5Phos][mU][Ps][fG][Ps][mG][fA][mU][fU][mU][fG][mG][fA][mG][fA][mA][fU][mG][Ps][fA][Ps] |
| | 153a | [mA][Ps][fC][Ps][rC][fG][Ps][mG][Ps][fU][mA][fC][mU][fC][mC][fU][mU][fG][mU][fU][Ps] |
| | S-A28s | [mG][Ps][fA][Ps][3XGaINAc] |
| 635 | A28a | [5Phos][mU][Ps][fC][Ps][mA][fA][mC][fA][mA][fG][mG][fA][mG][fU][mA][[C][mC][Ps][fC][Ps] |
| | s-TMP | [mG][Ps][fG][Ps][rG][fC][Ps][mA][Ps][fU][mU][fC][mU][fC][mC][fA][mA][fA][mU][fC][Ps] |
| | RSS | [mC][Ps][fA][Ps][3xGaINAc] |
| | 153S | |
| 636 | TMP RSS | [5Phos][mU][Ps][fA][Ps][mA][fA][mG][fG][mG][fC][mA][fG][mC][fU][mG][fA][mG][Ps][fC][Ps] |
| | 94as- | [mU][Ps][fC][Ps][rA][fG][Ps][mG][Ps][fU][mA][fC][mU][fC][mC][fU][mU][fG][mU][fU][Ps] |
| | A28S | [mG][Ps][fA][Ps][3XGaINAc] |
| 637 | A28a | [5Phos][mU][Ps][fC][Ps][mA][fA][mC][fA][mA][fG][mG][fA][mG][fU][mA][fC][mC][Ps][fC][Ps] |
| | S-TMP | [mG][Ps][fG][Ps][rG][fC][Ps][mU][Ps][fC][mA][fG][mC][fU][mG][fC][mC][fC][mU][fU][Ps] |
| | RSS 94S | [mU][Ps][fA][Ps][3xGaINAc] |
| 638 | TMP RSS | [5Phos][mU][Ps][fA][Ps][mC][fG][mC][fA][mG][fU][mU][fU][mC][fU][mC][fU][mC][Ps][fA][Ps] |
| | 171a | [mU][Ps][fC][Ps][C][fG][Ps][mG][Ps][fU][mA][fC][mU][fC][mC][fU][mU][fG][mU][fU][Ps][mG] |
| | S-A28s | [Ps][fA][Ps][3XGaINAc] |
| 639 | A28a | [5Phos][mU][Ps][fC][Ps][mA][fA][mC][fA][mA][fG][mG][fA][mG][fU][mA][fC][mC][Ps][fC][Ps] |
| | S-TMP | [mG][Ps][fG][Ps][rG][fG][Ps][mA][Ps][fG][mA][fG][mA][fA][mA][fC][mU][fG][mC][fG][Ps] |
| | RSS | [mU][Ps][fA][Ps][3xGalNAc] |
| | 171s | |
| 640 | TMP RSS | [5Phos][mU][Ps][fG][Ps][mC][fA][mG][fC][mU][fU][mU][fA][mU][fU][mC][fC][mA][Ps][fA][Ps] |
| | 121a | [mA][Ps][fG][Ps][G][fG][Ps][mG][Ps][fU][mA][fC][mU][fC][mC][fU][mU][fG][mU][fU][Ps][mG] |
| | S-A28s | [Ps][fA][Ps][3XGaINAc] |
| 641 | A28a | [Phos][mU][Ps][fC][Ps][mA][fA][mC][fA][mA][fG][mG][fA][mG][fU][mA][C][mC][Ps][fC][Ps] |
| | S-TMP | [mG][Ps][fG][Ps][rG][fU][Ps][mG][Ps][fG][mA][fA][mU][fA][mA][fA][mG][fC][mU][fG][Ps] |
| | RSS | [mC][Ps][fA][Ps][3xGaINAc] |
| | 121s | |
| 642 | TMP RSS | [5Phos][mU][Ps][fC][Ps][mA][fG][mU][fU][mU][C][mU][fC][mU][[C][mA][fU][mC][Ps][fC][Ps] |
| | 32as- | [mA][Ps][fG][Ps][G][fG][Ps][mG][Ps][fU][mA][fC][mU][fC][mC][fU][mU][fG][mU][fU][Ps] |
| | A28s | [mG][Ps][fA][Ps][3XGaINAc] |
| 643 | A28a | [5Phos][mU][Ps][fC][Ps][mA][fA][mC][fA][mA][fG][mG][fA][mG][fU][mA][fC][mC][Ps][C][Ps] |
| | S-TMP | [mG][Ps][fG][Ps][rG][fG][Ps][mA][Ps][fU][mG][fA][mG][fA][mG][fA][mA][fA][mC][fU][Ps] |
| | RSS 32s | [mG][Ps][fA][Ps][3xGaINAc] |

Note
each of the above constructs may or may not have a phosphate modification at the 5′ end group. In certain embodiments, e.g. in the case of a muRNA, the 3′ terminus of the antisense sequence may be unmodified and not carry a phosphorothioate but a phosphate. Experimental denotation “as” means antisense strand and “s” means sense strand.

Table 7b shows unmodified TMPRSS6-APOC3 muRNA constructs of the present disclosure in their double stranded form (each strand of the two strands is in a separate line for the respective SEQ ID NO).

| SEQ | | |
|---|---|---|
| ID | Experimental | |
| NO: | denotation | muRNA construct |
| 644 | TMPRSS153as- | UGGAUUUGGAGAAUGAACCGGUACUCCUUGU |
| | A28s | UGA |
| 645 | A28as- | UCAACAAGGAGUACCCGGGCAUUCUCCAAAU |
| | TMPRSS153S | CCA |
| 646 | TMPRSS94as- | UAAAGGGCAGCUGAGCUCAGGUACUCCUUGU |
| | A28S | UGA |
| 647 | A28as- | UCAACAAGGAGUACCCGGGCUCAGCUGCCCU |
| | TMPRSS94S | UUA |
| 648 | TMPRSS171as- | UACGCAGUUUCUCUCAUCCGGUACUCCUUGU |
| | A28s | UGA |
| 649 | A28as- | UCAACAAGGAGUACCCGGGGAGAGAAACUGC |
| | TMPRSS171s | GUA |
| 650 | TMPRSS121as- | UGCAGCUUUAUUCCAAAGGGGUACUCCUUGU |
| | A28s | UGA |
| 651 | A28as- | UCAACAAGGAGUACCCGGGUGGAAUAAAGCU |
| | TMPRSS121s | GCA |
| 652 | TMPRSS32as- | UCAGUUUCUCUCAUCCAGGGGUACUCCUUGU |
| | A28s | UGA |
| 653 | A28as- | UCAACAAGGAGUACCCGGGGAUGAGAGAAAC |
| | TMPRSS32s | UGA |

Note
each of the above constructs may or may not have a phosphate modification at the 5′ end group. In certain embodiments, e.g. in the case of a muRNA, the 3′ terminus of the antisense sequence may be unmodified and not carry a phosphorothioate but a phosphate. Experimental denotation “as” means antisense strand and “s” means sense strand.

While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the Examples described above may be combined with aspects of any of the other Examples described to form further Examples.

It will be understood that the above description of a optional embodiment is given by way of example only and that various modifications may be made by those skilled in the art. What has been described above includes Examples of one or more embodiments. It is, of course, not possible to describe every conceivable modification and alteration of the above compounds, compositions or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

### EXAMPLES

The following Examples illustrate certain embodiments of the present disclosure and are not limiting. Moreover, where specific embodiments are provided, the inventors have contemplated generic application of those specific embodiments. For example, disclosure of a construct having a particular motif or modification patterns provides reasonable support for additional constructs having the same or similar motif or modification patterns.

The syntheses of the RNAi constructs, e.g., muRNA constructs, disclosed herein have been conducted using synthesis methods known to the person skilled in the art, such as synthesis methods disclosed in https://en.wikipedia.org/wiki/Oligonucleotide_synthesis {retrieved on 15 Mar. 2022}, wherein the methods disclosed on this website are incorporated by reference herein in their entirety. The only difference to the synthesis method disclosed in this reference is that GalNAc phosphoramidite immobilized on a support is used in the synthesis method during the first synthesis step

### Example 1: In Vivo Study

The muRNA construct (two strands) composed of the sequences (strands) listed in Tables 5a and 5b (SEQ ID NOs 670 and 672) was used for the following in vivo study of this example. All future forms (like “will be”) in the following text are to be considered as past tense, as the study has already been carried out and the wording is just taken from the original study protocol.

Dose and Duration Response of Dual Targeting muRNA in humanized liver-uPA-SCID mice (PXB) model and normal mice, non-GLP

### 1. Study Objective(s)

The objective of this non-GLP study is to evaluate the dose and duration response of GalNAc-siRNA conjugated dual targeting (APOC3 and TMPRSS6) muRNA construct in humanized liver-uPA-SCID (PXB) mice and normal mice. The compound(s) will be administered subcutaneously, and the mice will be survived for up to 49 days.

Prior to necropsy, plasma and serum will be collected. At necropsy, 3 liver biopsies (2 mm) per animal will be preserved in separate vials in RNAlater, flash frozen, and stored at-80° C. Three more liver biopsies (2 mm) will be taken, flash frozen in the same vial, and stored at −80°.

### 2. Test System Information

### 2.1. Animal Test

### 2.1.1. Common Name: Mouse

### 2.1.2. Breed/Class: Rodent—Mouse PXB and C57/BL6

2.1.3. Number of Animals (by gender): 40 Male PXB and 40 male C57/BL6 all naïve
2.1.4. Age Range: 14-19 weeks for PXB mice, 8 weeks for C57/BL6
2.1.5. Weight Range: Approx. 20 grams for all mice

### 2.2. Acclimation Period:

### 2.2.1. Duration:

All animals will be acclimated for a minimum period of five (5) days prior to release by the Attending veterinarian, at which time the overall health of the animals will be evaluated. Animals which are not released from acclimation will be treated accordingly and further evaluation will be performed prior to release. All records from the acclimation period will remain in the study file.

2.2.2. Required Medication and/or Vaccination:
All rodents received will come from a vendor that is certified to be free of any lethal parasites that may affect the facility's total colony.
All rodents must be accompanied by a sentinel report including statistical analysis.
Each shipment of rodents must be housed separately from others in the facility.

### 3. Study Design

### 3.1. Design Details

This study will have two type of mice, 40 PXB and 40 C57/BL6. Animals will be grouped by treatment type, dosage, and survival period. Each animal will be treated by subcutaneous injection of test material. (Note: that the injection must be given subcutaneously. The test articles will not be functional if the subcutaneous site is missed, and injection is given within the muscular region or test articles are injected into the vein/bloodstream). See Study Table 8 for details.
Prior to necropsy, the animals will be deeply anesthetized, and a terminal blood draw will be performed through the vena cava. Blood volume collected will be split evenly between a serum and plasma separation tube.

Note: serum and plasma will be used to measure protein, caution should be taken to avoid hemolysis or clot formation.

At necropsy, three 2 mm biopsy punches will be taken from the left, middle and right liver lobes, placed in separate vials, soaked in RNAlater for 15 minutes, flash frozen and stored at −80° C. Another three 2 mm liver biopsies from the left, middle and right liver lobes will be placed into one vial, flash frozen and stored at −80° C. The rest of the liver will be flash frozen and stored in 10 ml conical tubes at −80° C.

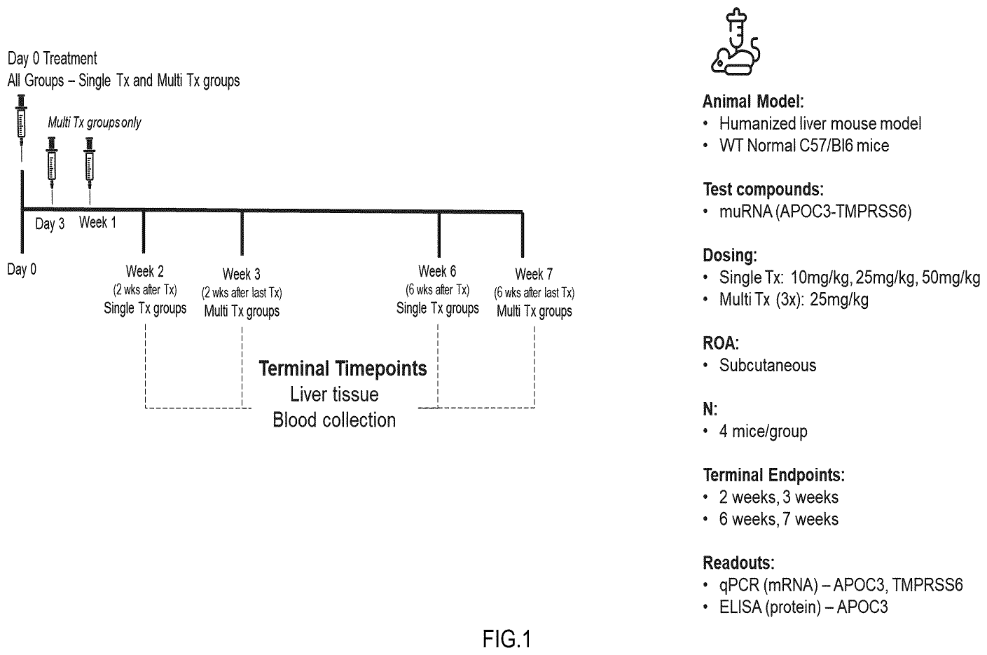

A schematic overview of the design of the in vivo study is shown in FIG. 1.

TABLE 8
Study Table

| | | | Treatment | | | |
|---|---|---|---|---|---|---|
| | Number | | Subcutaneous | | | |
| | of | Mouse | Injection | Survival | | Pre-Euthanasia |
| Group | Animals | Type | Day 0 | Days | Blood | and Necropsy |
| 1A | 4 | PXB | Control (PBS) | 14 | Plasma and | Pre-Euthanasia: |
| 1C | 4 | PXB | Control (PBS) | 42 | serum will be | Plasma and serum |
| 2A | 4 | PXB | muRNA (10 mg/kg) | 14 | collected for | collection. |
| 2B | 4 | PXB | muRNA (25 mg/kg) | 14 | all animals at | Necropsy: |
| 2C | 4 | PXB | muRNA (50 mg/kg) | 14 | necropsy. | 2 mm biopsy of |
| 2E | 4 | PXB | muRNA (25 mg/kg) | 42 | Separate | left, middle and |
| 3A | 4 | C57/BL6 | Control (PBS) | 14 | serum and | right liver lobes in |
| 3C | 4 | C57/BL6 | Control (PBS) | 42 | plasma in 2 | separate vials, in |
| 4A | 4 | C57/BL6 | muRNA (10 mg/kg) | 14 | tubes. | RNAlater for 15 |
| 4B | 4 | C57/BL6 | muRNA (25 mg/kg) | 14 | Send one | min, flash freeze |
| 4C | 4 | C57/BL6 | muRNA (50 mg/kg) | 14 | serum tube to | then stored at −80° C. |
| 4E | 4 | C57/BL6 | muRNA (25 mg/kg) | 42 | IDEXX for | 2 mm biopsy of |
| | | | | | lipid panel | left, middle and |
| | | | | | analysis. | right liver all in one |
| | | | | | Send 2 | vial, flash freeze |
| | | | | | Plasma and 1 | then stored at −80° C. |
| | | | | | serum tubes | Rest of liver, flash |
| | | | | | to Sponsor. | freeze then stored |
| | | | | | | at −80° C. |

| | | | Treatment | |
|---|---|---|---|---|
| | Number | | Subcutaneous | |
| | of | Mouse | Injection | Survival |
| Group | Animals | Type | Days 0, 3, 7 | Days |
| 1B | 4 | PXB | Control (PBS) | 21 |
| 1D | 4 | PXB | Control (PBS) | 49 |
| 2D | 4 | PXB | muRNA (25 mg/kg) | 21 |
| 2F | 4 | PXB | muRNA (25 mg/kg) | 49 |
| 3B | 4 | C57/BL6 | Control (PBS) | 21 |
| 3D | 4 | C57/BL6 | Control (PBS) | 49 |
| 4D | 4 | C57/BL6 | muRNA (25 mg/kg) | 21 |
| 4F | 4 | C57/BL6 | muRNA (25 mg/kg) | 49 |
| Spares | 4 | C57/BL6 | | |
| Total | 40 | PXB | | |
| | 44 | C57/BL6 | | |

### 3.2. Route of Administration

Subcutaneous injection in the scruff. An injection volume of 200 uL. (Note: that the injection must be given subcutaneously. The test articles will not be functional if the subcutaneous site is missed, and injection is given within the muscular region or test articles are injected into the vein/bloodstream).

### 4. Test Article and Ancillary Material Information

### 4.1. Test Drug 1:

4.1.1. Identification: muRNA (APOC3-TMPRSS6)

### 4.1.2. Manufacturer: Sirnaomics

4.1.3. Description: GalNAc-siRNA targeting human APOC3 mRNA and human TMPRSS6 mRNA (muRNA composed of the strands of Tables 5a and 5b).
4.1.4. Lot/Batch Number: Will be recorded on study materials form.
4.1.5. Expiration Date: Will be recorded on study materials form.

### 4.1.6. Storage Temperature: 4° C.

### 4.1.7. Bio-Hazard Status: None

### 4.1.8. MSDS*: TBD

### 4.1.9. Appearance: Clear Liquid

### 4.1.10. Dose Information: See Table 8

### 4.1.11. Residual Test Article Storage: None

### 5. Technical and Analytical Procedures.

### Blood Collection Prior to Necropsy:

Prior to necropsy, the animals will be deeply anesthetized, and a terminal blood draw will be performed through the vena cava. Blood volume collected will be split evenly between a serum and plasma separation tube. After separation the serum and plasma samples will be labeled in separate vials, flash frozen and stored at −80° C.

Note: serum and plasma will be used to measure protein, caution should be taken to avoid hemolysis or clot formation.

### Necropsy and Explant Procedure:

Note: Tissue samples will be taken using separate tools for each individual collection. Tissue harvesting tools will be changed for each tissue sample to prevent cross contamination.

A 2 mm biopsy punch will be taken from the left, middle and right liver lobes. Place biopsy samples into separate 2 ml Eppendorf tubes, with 1.5 ml RNAlater and let soak for 15 minutes, flash freeze then store at −80° C. Three more 2 mm biopsy samples will be taken of the left, middle and right liver lobes all placed together into one 2 ml Eppendorf tubes, flash freeze then store at −80° C. Remaining liver will be flash frozen and stored in 10 mL conical tubes at-80° C.

### 6. Results

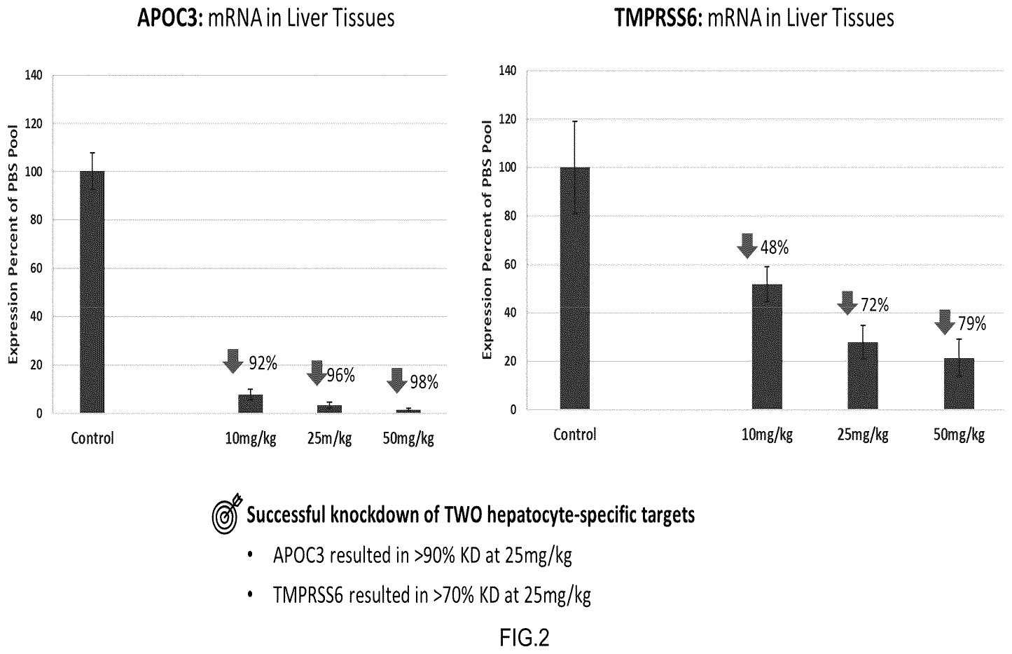

FIGS. 2 to 5 show performance as follows.

FIG. 2 shows knockdown of TMPRSS6 and APOC3 mRNA in liver tissue.

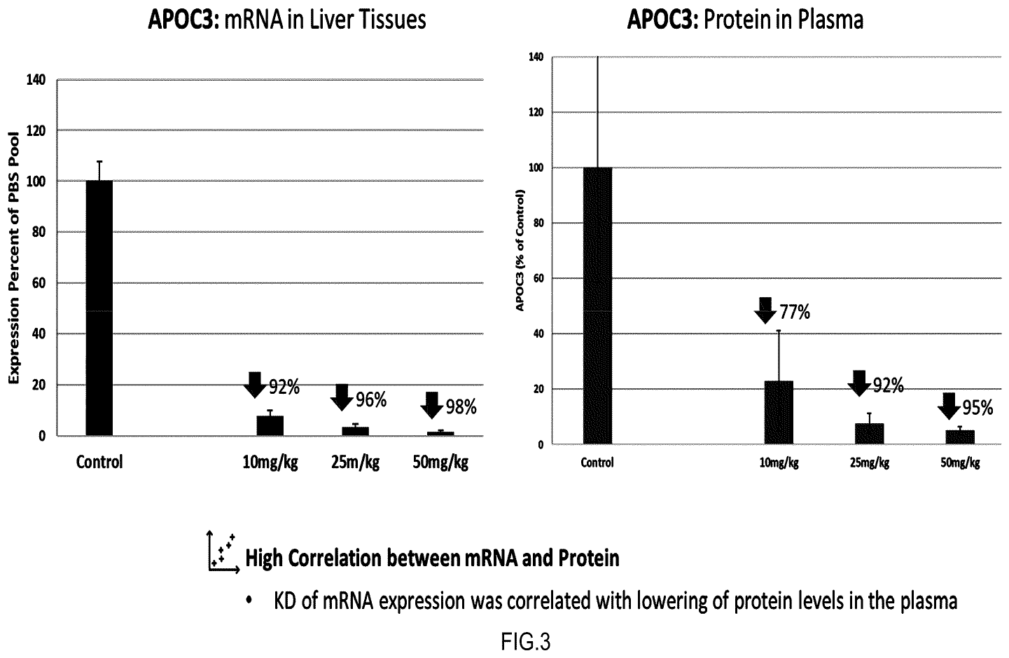

FIG. 3 shows a comparison of APOC3 mRNA knockdown in liver tissue with APOC3 protein knockdown in plasma, demonstrating a high correlation between the two parameters.

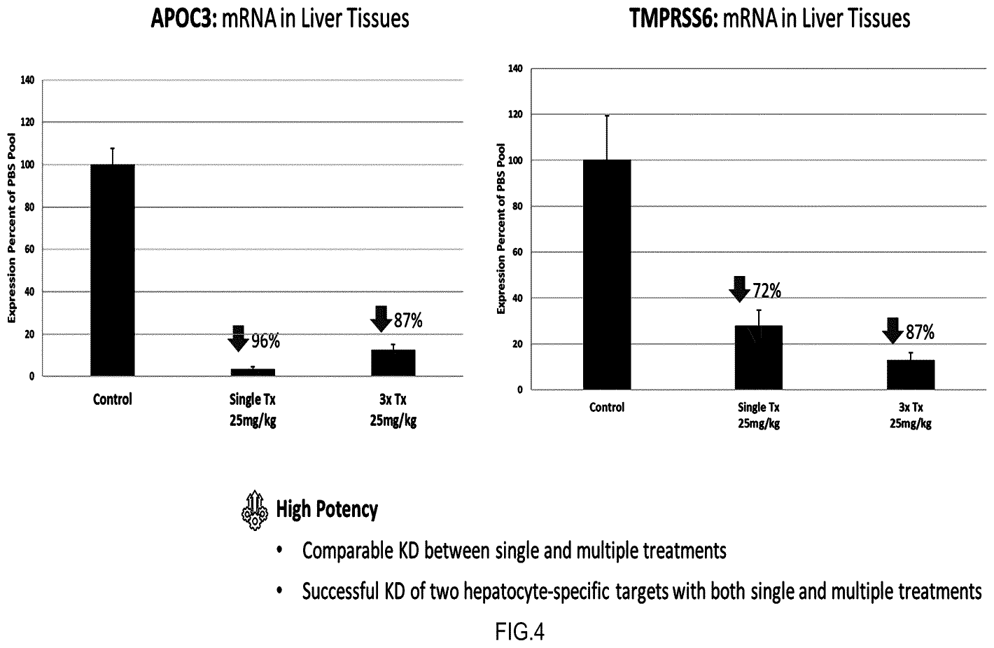

FIG. 4 compares single treatment with multiple treatment (see the study design in FIG. 1). Results are comparable, wherein a further increase of TMPRSS6 mRNA knockdown is observed for multiple treatment.

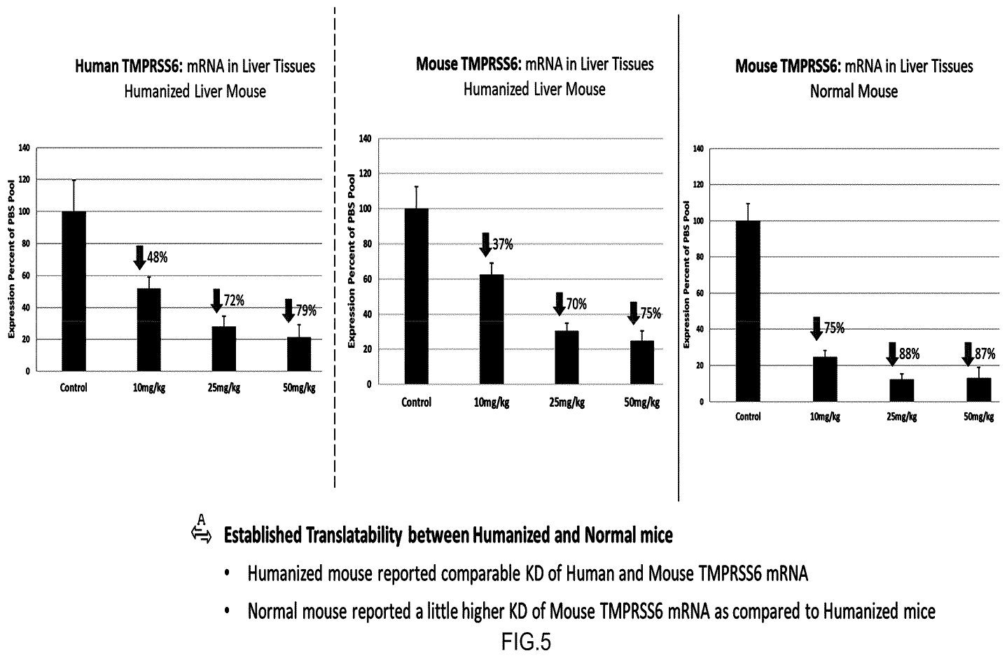

FIG. 5 compares the effect on TMPRSS6 mRNA levels in both normal mouse and mice with a humanized liver. The humanized mouse liver still retains a certain fraction of murine liver cells. Since a construct has been employed which is capable of knocking down both human and murine TMPRSS6, all three read-outs shown demonstrate knockdown of the respective TMPRSS6 mRNA.

Overall, concomitant and significant knockdown of both target genes could be demonstrated.

### Example 2: In Vitro Study

A seven step, fivefold dilution series of compounds was prepared in basal WEM from 2 μM to 0.000128 μM.

On the day of transfection, primary human hepatocytes were thawed in 45 mL of human OptiThaw (Sekisui XenoTech, K8000) and centrifuged down at 200 g for 5 minutes. Cells were resuspended in 2× complete WEM and counted. Cells were then plated in 50 μL of 2x complete WEM at 25,000 cells per well on 96 well type 1 rat tail collagen plates and allowed to rest and attach for four hours before transfection. After rest, 50 μL of each dilution was added to respective triplicates of the plated hepatocytes for a final dilution series of 1 μM down to 0.000064 μM in a volume of 100 uL 1× complete WEM.

72 hours post transfection, cells were harvested, and RNA isolated using the PureLink Pro 96 total RNA Purification Kit (ThermoFisher, 12173011A) according to the manufacturer protocol. Harvested RNA was assayed for TMPRSS6 or APOC3 expression via Taqman qPCR using the Luna Universal Probe One-Step RT-qPCR Kit (NEB, E3006). A qPCR assay was performed for each sample using a TMPRSS6 (Hs00542191_m1-FAM) or APOC3 TaqMan probe set (Hs00906501_g1-FAM) multiplexed with a common GAPDH VIC probe (ThermoFisher, 4326317E). Thermocycling and data acquisition was performed with an Applied Biosystems QuantStudio 3/5 Real-Time PCR System.

### Results

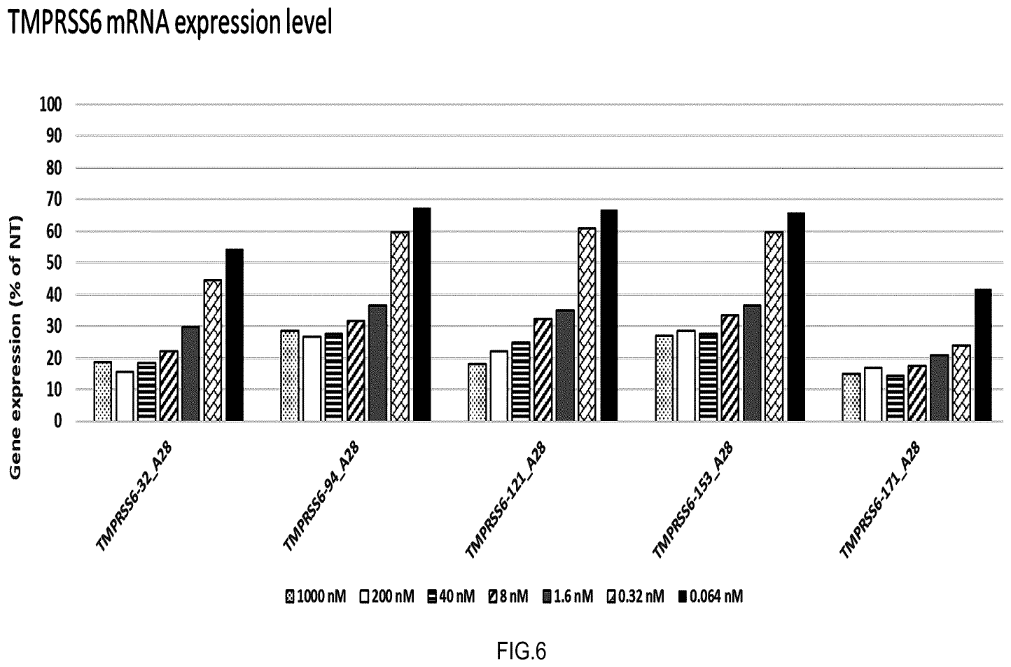

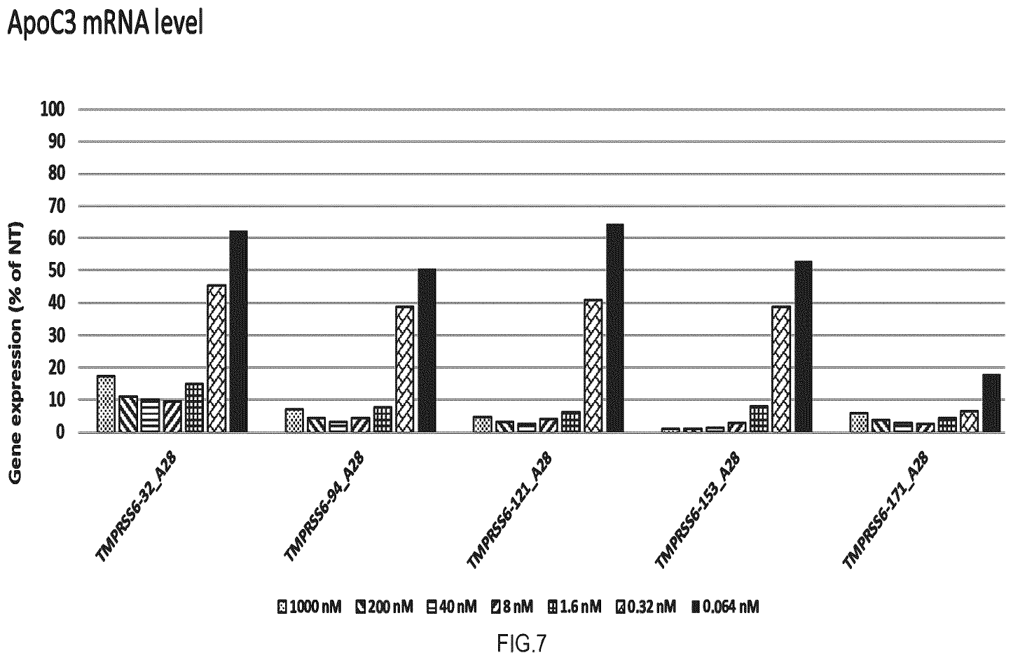

Tables 9a and 9b below show IC50 values (maximum knock down value at 1000 nM in %) for specific constructs as a result of the dose response assay for TMPRSS6 and APCO3, respectively. The constructs correspond to the ones in Table 7a in view of their experimental denotation. The results of the dose response assay are also shown in FIGS. 6 and 7, respectively.

TABLE 9a

| SEQ ID NOs constituting | Experimental | Max KD % at | IC50 |
|---|---|---|---|
| muRNA | Denotation | 1000 nM | (nM) |
| 642 and 643 | TMPRSS6-32_A28 | 81.4 | 0.022 |
| 636 and 637 | TMPRSS6-94_A28 | 71.6 | 0.215 |
| 640 and 641 | TMPRSS6-121_A28 | 81.8 | 0.163 |
| 634 and 635 | TMPRSS6-153_A28 | 72.9 | 0.222 |
| 638 and 639 | TMPRSS6-171_A28 | 85.1 | 0.001 |

TABLE 9b

| SEQ ID NOs constituting | Experimental | Max KD % at | IC50 |
|---|---|---|---|
| muRNA | Denotation | 1000 nM | (nM) |
| 642 and 643 | TMPRSS6-32_A28 | 82.7 | 0.162 |
| 636 and 637 | TMPRSS6-94_A28 | 92.9 | 0.081 |
| 640 and 641 | TMPRSS6-121_A28 | 95.3 | 0.126 |
| 634 and 635 | TMPRSS6-153_A28 | 99.0 | 0.087 |
| 638 and 639 | TMPRSS6-171_A28 | 94.2 | 0.005 |

The results of the in vitro dose studies are also illustrated in FIGS. 6 and 7 for the reduction of gene expression of TMPRSS6 and APOC3 mRNA levels, respectively.

1. A nucleic acid construct comprising:
(a) a first nucleic acid sequence that is complementary to a first portion of an RNA which is transcribed from a targeted TMPRSS6 gene;
(b) a second nucleic acid sequence that is complementary to a second portion of an RNA which is transcribed from a targeted APOC3 gene;
(c) a third nucleic acid sequence that is at least complementary to the first nucleic acid portion of (a), so as to form a first nucleic acid duplex region therewith;
(d) a fourth nucleic acid sequence that is partially complementary to the second nucleic acid portion of (b), so as to form a second nucleic acid duplex region therewith,
wherein the first nucleic acid sequence of (a) is directly linked to the fourth nucleic acid sequence of (d) and the second nucleic acid of (b) is directly linked to the third nucleic acid sequence of (c);
wherein the construct contains labile sites such that subsequent to in vivo administration the construct is cleaved at said labile sites to yield at least first and second discrete nucleic acid targeting molecules that respectively target the RNA portions transcribed from the targeted genes of (a) and (b);
wherein the
wherein (i) the first nucleic acid targeting molecule modulates expression of the target gene of (a), and comprises, or is derived from, the first nucleic acid portion of (a), and (ii) the second nucleic acid targeting molecule modulates expression of the targeted gene of (b), and comprises, or is derived from, the second nucleic acid portion of (b).

2-4. (canceled)

5. The construct according to claim 1, wherein the labile sites comprise unmodified nucleotides.

6-8. (canceled)

9. The construct according to claim 1, wherein
(a) the first nucleic acid sequence is selected from the group consisting of SEQ ID NOs: 1 to 3;
(b) the second nucleic acid sequence is selected the group consisting of SEQ ID NOs: 8 to 14, and SEQ ID NO: 29;
(c) the third nucleic acid sequence is selected from the group consisting of SEQ ID NOs: 15 to 17; and/or
(d) the fourth nucleic acid sequence is sequence selected from the group consisting of SEQ ID NOs: 22 to 28, and SEQ ID NO: 30.

10. (canceled)

11. The construct according to claim 1, wherein the first and the fourth nucleic acid portions have the nucleobase sequences selected from the group consisting of SEQ ID NOs: 1 and 24; 1 and 22; 1 and 25; 1 and 26; 1 and 28; 1 and 30; 3 and 24; 3 and 22; 3 and 25; 3 and 26; 3 and 28; 3 and 30; 2 and 24; 2 and 22; 2 and 25; 2 and 26; 2 and 28; 2 and 30, respectively, and
wherein the second and third nucleic acid portions have the nucleobase sequences selected from the group consisting of SEQ ID NOs: 10 and 15; 8 and 15; 11 and 15; 12 and 15; 14 and 15; 29 and 15; 10 and 16; 8 and 16; 11 and 16; 12 and 16; 14 and 16; 29 and 16; 10 and 17; 8 and 17; 11 and 17; 12 and 17; 14 and 17; 29 and 17, respectively, and optionally, 10 and 15.

12-21. (canceled)

22. The construct according to claim 1, wherein the first nucleic acid portion of (a) and the second nucleic acid portion of (b) have a length of 18 or 19 nucleotides and the third nucleic acid portion of (c), and the fourth nucleic acid portion of (d) have a length of 14 or 15.

23-27. (canceled)

28. The construct according to claim 1, which further comprises one or more ligands.

29-36. (canceled)

37. The construct according to claim 28, which comprises one, two, or three N-Acetyl-Galactosamine moieties.

38-39. (canceled)

40. The construct according to claim 37, wherein the ligand has the following structure:

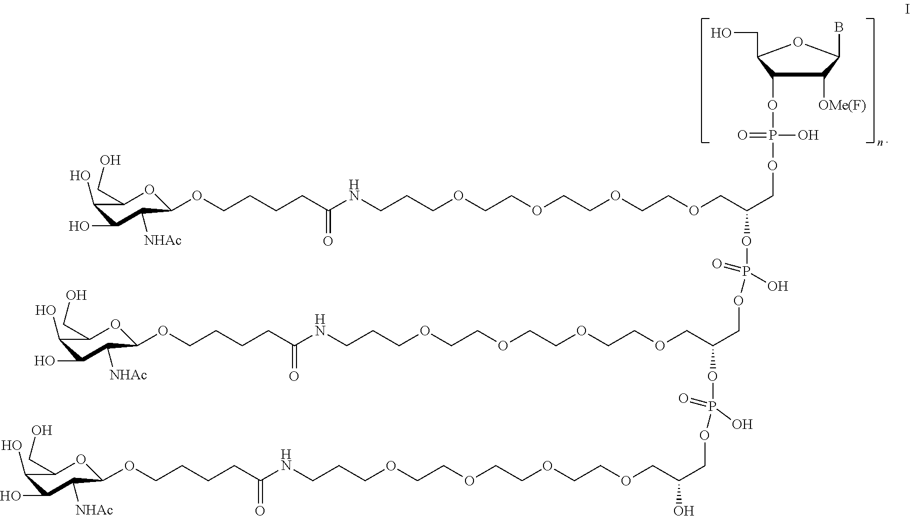

41. (canceled)

42. The construct according to claim 1, which comprises 1 to 15 phosphorothioate or phosphorodithioate internucleotide linkages.

43-46. (canceled)

47. The construct according to claim 1, wherein at least one nucleotide is 2′-modified.

49-61. (canceled)

62. The construct according to claim 47, wherein the 2′ modified sugar is a 2′-O-methyl modified sugar or a 2′ O allyl modified sugar.

63-69. (canceled)

70. The construct according to claim 5, wherein all remaining nucleotides other than the labile sites contain either 2′-O-methyl modifications or 2′-F modifications in ribose moieties.

71-72. (canceled)

73. The construct according to claim 1, wherein
(a) the first nucleic acid sequence comprises SEQ ID No. 654;
(b) the second nucleic acid sequence is selected from the group consisting of SEQ ID Nos. 655 to 661;
(c) the third nucleic acid sequence comprises SEQ ID No. 662; and/or
(d) the fourth nucleic acid sequence is selected from the group consisting of SEQ ID Nos. 663 to 669.

74. The construct according to claim 1, wherein the construct comprises two strands, wherein the first strand comprises SEQ ID No. 670 or 671, and the second strand comprises SEQ ID No. 672; or
the first and second strands are jointly selected from the group consisting of SEQ ID NOs: 634, 635, 636, 637, 638, 639, 640, 641, 642, and 643; or
the first and second strands are jointly selected from the group consisting of SEQ ID NOs: 644, 645, 646, 647, 648, 649, 650, 651, 652 and 653.

75. The construct of claim 74, wherein first and second strands are as shown below:
(SEQ ID No. 670)
[mU][#][fG][#][mU][fA][mC][fC][mC][fU][mA][fG][mG]
[fA][mA][fA][mU][#][fA][#][mC][#][fC][#][rA][mG]
[#][fU][#][mA][fC][mU][fC][mC][fU][mU][fG][mU][fU]
[#][mG][#][A][#][3XGalNAc];
and
(SEQ ID No. 672)
[mU][#][fC][#][mA][fA][mC][fA][mA][fG][mG][fA][mG]
[fU][mA][fC][#][mC][#][fC][#][mG][#][fG][#][rG]
[fA][#][mU][#][fU][mU][fC][mC][fU][mA][fG][mG][fG]
[mU][fA][#][mC][#][fA][#][3XGaINAc],
wherein [mN], N being any nucleoside, designates 2′-OMe; [fN], N being any nucleoside, designates: 2′-F; [rA], N being any nucleoside, designates: 2′-OH; [#] designates a phosphorothioate connecting two adjacent nucleosides; and [3XGalNAc] designates the following ligand, wherein the strand to which the ligand is bound is shown in square brackets:

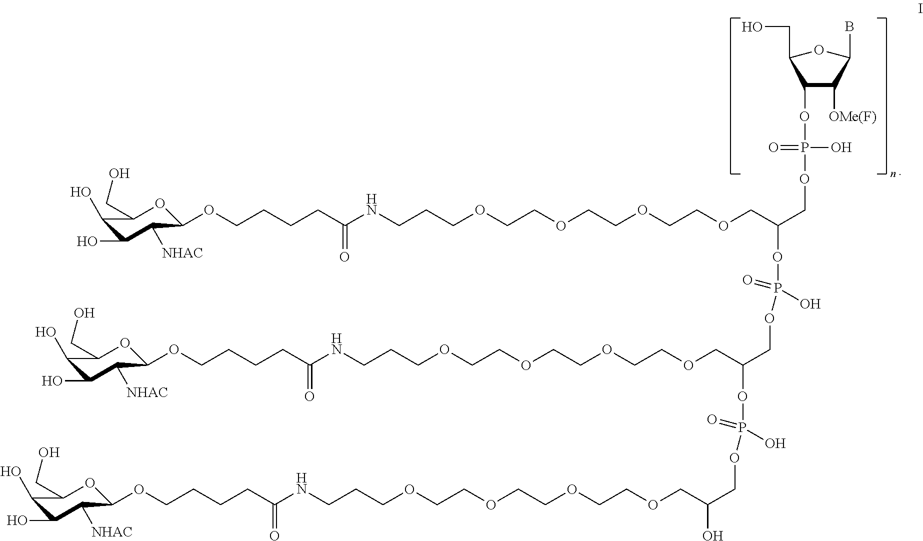

76-83. (canceled)

84. The construct of claim 1, wherein
(a) the first nucleic acid portion is selected from the group consisting of SEQ ID NOs: 465, 527, 553, 585, and 603;
(b) the second nucleic acid portion is selected from the group consisting of SEQ ID Nos. 655 to 661);
(c) the third nucleic acid portion comprises 14 or 15, contiguous nucleotides complementary to the corresponding part of the first nucleic acid portion; and/or
(d) the fourth nucleic acid portion is selected from the group consisting of SEQ ID Nos. 663 to 669).

85-87. (canceled)

88. The construct according to claim 1, wherein the first nucleic acid portion is selected from the group consisting of SEQ ID NOs: 65, 127, 153, 185, and 203 and the third nucleic acid portion is selected from the group consisting of SEQ ID NOs: 265, 327, 353, 385, and 403.

89-91. (canceled)

92. A pharmaceutical composition comprising a construct according to claim 1, and a physiologically acceptable excipient, diluent, antioxidant, and/or preservative.

93-103. (canceled)

104. A method of treating a disease or disorder comprising administering a construct according to claim 1, to an individual in need of treatment wherein the disease or disorder is
(a) a TMPRSS6-associated disease or disorder; a disease or disorder associated with excess accumulation of iron and/or requiring reduction of iron levels such as transfusional iron overload, excess parenteral iron supplement, and excess dietary iron intake; a disease or disorder selected from blood disorders such as hemochromatosis, anaemia, thalassaemia, porphyria, and hemosiderosis; bone marrow failure syndromes and myelodysplasia; neurological disorders such as Parkinson's disease, Alzheimer's disease, and Friedreich's ataxia; and/or chronic liver diseases;
and/or
(b) an APOC3-associated disease or disorder, or a disease or disorder requiring reduction of APOC3 expression levels, the disease or disorder optionally being selected from dyslipidemia including mixed dyslipidemia; hyperchylomicronemia including familial hyperchylomicronemia; hypertriglyceridemia, optionally severe hypertriglyceridemia and/or hypertriglyceridemia with blood triglyceride levels above 500 mg/dl; inflammation including low-grade inflammation; atherosclerosis; atherosclerotic cardiovascular diseases (ASCVD) including major adverse cardiovascular events (MACE) such as myocardial infarction, stroke and peripheral arterial disease; and pancreatitis including acute pancreatitis.

105. The method according to claim 104, wherein the construct is administered subcutaneously or intravenously to the individual.

106-113. (canceled)
